SEQUENCE LISTING (81 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - DNA, 22 bp, Chlamydomonas reinhardtii
  source 1..22: mol_type = genomic DNA
gaaactgtctcacgacgttttg

SEQ ID NO: 3 - DNA, 22 bp, Chlamydomonas reinhardtii
  source 1..22: mol_type = genomic DNA
caaaacgtcgtgagacagtttc

SEQ ID NO: 4 - DNA, 22 bp, Chlamydomonas reinhardtii
  source 1..22: mol_type = genomic DNA
caaactgtcgtgagacagtttg

SEQ ID NO: 5 - DNA, 22 bp, Chlamydomonas reinhardtii
  source 1..22: mol_type = genomic DNA
caaactgtctcacgacagtttg

SEQ ID NO: 6 - protein, 170 aa, Monomastix sp.
MTTKNTLQPTEAAYIAGFLDGDGSIYAKLIPRPDYKDIKYQVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSP

SEQ ID NO: 7 - DNA, 22 bp, Monomastix sp.
  source 1..22: mol_type = genomic DNA
cagaacgtcgtgagacagttcc

SEQ ID NO: 8 - DNA, 22 bp, Monomastix sp.
  source 1..22: mol_type = genomic DNA
ggaactgtctcacgacgttctg

SEQ ID NO: 9 - protein, 235 aa, Saccharomyces cerevisiae
MKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 10 - DNA, 18 bp, Saccharomyces cerevisiae
  source 1..18: mol_type = genomic DNA
ttaccctgttatccctag

SEQ ID NO: 11 - DNA, 18 bp, Saccharomyces cerevisiae
  source 1..18: mol_type = genomic DNA
ctagggataacagggtaa

SEQ ID NO: 12 - protein, 218 aa, Chlamydomonas moewusii
MSNFILKPGEKLPQDKLEELKKINDAVKKTKNFSKYLIDLRKLFQIDEVQVTSESKLFLAGFLEGEASLNISTKKLATSKFGLVVDPEFNVTQHVNGVKVLYLALEVFKTGRIRHKSGSNATLVLTIDNRQSLEEKVIPFYEQYVVAFSSPEKVKRVANFKALLELFNNDAHQDLEQLVNKILPIWDQMRKQQGQSNEGFPNLEAAQDFARNYKKGIK

SEQ ID NO: 13 - DNA, 22 bp, Chlamydomonas moewusii
  source 1..22: mol_type = genomic DNA
ataacggtcctaaggtagcgaa

SEQ ID NO: 14 - DNA, 22 bp, Chlamydomonas moewusii
  source 1..22: mol_type = genomic DNA
ttcgctaccttaggaccgttat

SEQ ID NO: 15 - DNA, 22 bp, Human immunodeficiency virus 1
  source 1..22: mol_type = genomic DNA
gaagagctcatcagaacagtca

SEQ ID NO: 16-21 - skipped (6 entries)

SEQ ID NO: 22 - DNA, 22 bp, synthetic construct
  misc_feature 1..22: note = Synthetic oligonucleotide
  source 1..22: mol_type = other DNA
tgcggtgtccggcgacagcctg

SEQ ID NO: 23 - DNA, 22 bp, Homo sapiens
  source 1..22: mol_type = genomic DNA
ctgggagtctcaggacagcctg

SEQ ID NO: 24 - DNA, 22 bp, Homo sapiens
  source 1..22: mol_type = genomic DNA
ccaggtgtctctggactcctcc

SEQ ID NO: 25 - DNA, 22 bp, Homo sapiens
  source 1..22: mol_type = genomic DNA
gaaaatatcattggtgtttcct

SEQ ID NO: 26 - DNA, 22 bp, Homo sapiens
  source 1..22: mol_type = genomic DNA
aaccctctccagtgagatgcct

SEQ ID NO: 27 - DNA, 22 bp, Homo sapiens
  source 1..22: mol_type = genomic DNA
gacctcgtcctccgactcgctg

SEQ ID NO: 28 - DNA, 22 bp, Herpes simplex virus
  source 1..22: mol_type = genomic DNA
ctcgatgtcggacgacacggca

SEQ ID NO: 29 - DNA, 22 bp, Bacillus anthracis
  source 1..22: mol_type = genomic DNA
acaagtgtctatggacagttta

SEQ ID NO: 30 - DNA, 22 bp, Variola virus
  source 1..22: mol_type = genomic DNA
aaaactgtcaaatgacatcgca

SEQ ID NO: 31 - DNA, 22 bp, Epstein-Barr virus
  source 1..22: mol_type = genomic DNA
cggggtctcgtgcgaggcctcc

SEQ ID NO: 32 - DNA, 22 bp, Arabidopsis thaliana
  source 1..22: mol_type = genomic DNA
cactaactcgtatgagtcggtg

SEQ ID NO: 33 - DNA, 22 bp, Arabidopsis thaliana
  source 1..22: mol_type = genomic DNA
tgcctcctctagagacccggag

SEQ ID NO: 34 - DNA, 22 bp, Nicotiana tabacum
  source 1..22: mol_type = genomic DNA
taaaatctctaaggtctgtgca

SEQ ID NO: 35 - DNA, 22 bp, Nicotiana tabacum
  source 1..22: mol_type = genomic DNA
caagaattcaagcgagcattaa

SEQ ID NO: 36 - DNA, 22 bp, Saccharomyces cerevisiae
  source 1..22: mol_type = genomic DNA
ttagatgacaagggagacgcat

SEQ ID NO: 37 - protein, 38 aa, Chlamydomonas reinhardtii
PGSVGGLSPSQASSAASSASSSPGSGISEALRAGATKS

SEQ ID NO: 38 - protein, 9 aa, synthetic construct
  REGION 1..9: note = Synthetic polypeptide
MAPKKKRKV

SEQ ID NO: 39 - protein, 114 aa, Saccharomyces cerevisiae
ANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKE

SEQ ID NO: 40 - protein, 78 aa, Herpes simplex virus
TAPITDVSLVDELRLDGEEVDMTPADALDDFDLEMLGDVESPSPGMTHDPVSYGALDVDDFEFEQMFTDALGIDDFGG

SEQ ID NO: 41 - protein, 63 aa, Chlamydomonas reinhardtii
VSVTFEDVAVLFTRDEWKKLDLSQRSLYREVMLENYSNLASMAGFLFTKPKVISLLQQGEDPW

SEQ ID NO: 42 - DNA, 22 bp, Homo sapiens
  source 1..22: mol_type = genomic DNA
aatggagacgcaagagagggag

SEQ ID NO: 43 - protein, 360 aa, synthetic construct
  REGION 1..360: note = Synthetic polypeptide
MAPKKKRKVIMNTKYNKEFLLYLAGFVDGDGSIIAAIDPQQNYKFKHSLRLRFTVTQKTQRRWFLDKLVDEIGVGYVRDRGSVSDYQLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGATKSKEFLLYLAGFVDGDGSIKAQIRPRQSCKFKHELELEFQVTQKTQRRWFLDKLVDEIGVGYVYDRGSVSDYILSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 44 - protein, 360 aa, synthetic construct
  REGION 1..360: note = Synthetic polypeptide
MAPKKKRKVIMNTKYNKEFLLYLAGFVDGDGSIIAAIDPQQNYKFKHSLRLRFTVTEKTQRRWFLDKLVDEIGVGYVRDRGSVSDYQLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGATKSKEFLLYLAGFVDGDGSIKAQIRPRQSCKFKHELELEFQVTEKTQRRWFLDKLVDEIGVGYVYDRGSVSDYILSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 45 - DNA, 22 bp, synthetic construct
  misc_feature 1..22: note = Synthetic oligonucleotide
  source 1..22: mol_type = other DNA
aggcatctcgtacgagatgcct

SEQ ID NO: 46 - protein, 245 aa, synthetic construct
  REGION 1..245: note = Synthetic polypeptide
MAPKKKRKVIMNTKYNKEFLLYLAGFVDGDGSIKAQIKPEQNRKFKHRLELTFQVTEKTQRRWFLDKLVDEIGVGYVYDRGSVSDYILSEIKPLHNFLTQLQPFLKLKQKQANLVLKIIEQLPSAKESPDKFLEVCTWVDQIAALNDSKTRKTTSETVRAVLDSLSEKKKSSPGSSGSSGSSVSVTFEDVAVLFTRDEWKKLDLSQRSLYREVMLENYSNLASMAGFLFTKPKVISLLQQGEDPW

SEQ ID NO: 47 - DNA, 442 bp, synthetic construct
  misc_feature 1..442: note = Synthetic polynucleotide
  source 1..442: mol_type = other DNA
gccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactaga

SEQ ID NO: 48 - protein, 9 aa, synthetic construct
  REGION 1..9: note = Synthetic polypeptide
LAGLIDADG

SEQ ID NO: 49 - protein, 9 aa, synthetic construct
  REGION 1..9: note = Synthetic polypeptide
GSSGSSGSS

SEQ ID NO: 50 - protein, 15 aa, synthetic construct
  REGION 1..15: note = Synthetic polypeptide
  REGION 1..15: note = This peptide may encompass 1 to 5 'Gly-Ser-Ser' repeating units
GSSGSSGSSGSSGSS

SEQ ID NO: 51 - protein, 6 aa, synthetic construct
  REGION 1..6: note = Synthetic polypeptide
HHHHHH

SEQ ID NO: 52 - protein, 4 aa, synthetic construct
  REGION 1..4: note = Synthetic polypeptide
CCHH

SEQ ID NO: 53 - protein, 4 aa, synthetic construct
  REGION 1..4: note = Synthetic polypeptide
WRPW

SEQ ID NO: 54-80 - skipped (27 entries)

SEQ ID NO: 81 - protein, 5 aa, Monomastix sp.
IGSTH

(19) United States
(12) Patent Application Publication
(10) Pub. No.: US 2025/0346874 A1
(43) Pub. Date: Nov. 13, 2025

(54) FUSION MOLECULES OF RATIONALLY-DESIGNED DNA-BINDING PROTEINS AND EFFECTOR DOMAINS

(71) Applicant: Duke University, Durham, NC (US)

(72) Inventors: Derek Jantz, Durham, NC (US); Michael G. Nicholson, Chapel Hill, NC (US); James Jefferson Smith, Morrisville, NC (US)

(73) Assignee: Duke University, Durham, NC (US)

(21) Appl. No.: 19/029,117

(22) Filed: Jan. 17, 2025

Related U.S. Application Data
(63) Continuation of application No. 18/352,037, filed on Jul. 13, 2023, now abandoned.
Application No. 18/352,037 is a continuation of application No. 17/224,054, filed on Apr. 6, 2021, now abandoned.
Application No. 17/224,054 is a continuation of application No. 17/107,414, filed on Nov. 30, 2020, now abandoned.
Application No. 17/107,414 is a continuation of application No. 16/658,987, filed on Oct. 21, 2019, now abandoned.
Application No. 16/658,987 is a continuation of application No. 15/666,425, filed on Aug. 1, 2017, now abandoned.
Application No. 15/666,425 is a continuation of application No. 14/679,733, filed on Apr. 6, 2015, now abandoned.
Application No. 14/679,733 is a continuation of application No. 13/623,017, filed on Sep. 19, 2012, now abandoned.
Application No. 13/623,017 is a continuation-in-part of application No. 13/223,852, filed on Sep. 1, 2011, now abandoned.
Application No. 13/223,852 is a continuation of application No. 11/583,368, filed on Oct. 18, 2006, now Pat. No. 8,021,867.
Application No. 13/623,017 is a continuation-in-part of application No. 12/914,014, filed on Oct. 28, 2010, now abandoned.
Application No. 12/914,014 is a continuation of application No. PCT/US2009/041796, filed on Apr. 27, 2009.
(60) Provisional application No. 60/727,512, filed on Oct. 18, 2005.
(60) Provisional application No. 61/048,499, filed on Apr. 28, 2008.

(51) Int. Cl. C12N 9/22; A61K 48/00; C07K 14/47; C12N 15/90

(52) U.S. Cl. CPC C12N 9/22; C07K 14/4702; C07K 14/4703; C12N 15/907; A61K 48/00; A61K 48/005; C07K 2319/09; C07K 2319/71; C07K 2319/80; C07K 2319/81

(57) ABSTRACT

Targeted transcriptional effectors (transcription activators and transcription repressors) derived from meganucleases are described. Also described are nucleic acids encoding same, and methods of using same to regulate gene expression. The targeted transcriptional effectors can comprise (i) a meganuclease DNA-binding domain lacking endonuclease cleavage activity that binds to a target recognition site; and (ii) a transcription effector domain.

### REFERENCE TO RELATED APPLICATIONS

This application is a Continuation of U.S. patent application Ser. No. 17/107,414, filed Nov. 30, 2020, which is a Continuation of U.S. patent application Ser. No. 16/658,987, filed Oct. 21, 2019, which is a Continuation of U.S. patent application Ser. No. 15/666,425, filed Aug. 1, 2017, which is a Continuation of U.S. patent application Ser. No. 14/679,733, filed Apr. 6, 2015, which is a Continuation of U.S. patent application Ser. No. 13/623,017, filed on Sep. 19, 2012 which is a Continuation-In-Part of U.S. patent application Ser. No. 12/914,014, filed Oct. 28, 2010, which is a Continuation of International Application PCT/US09/41796, filed Apr. 27, 2009, which claims the benefit of priority to U.S. Provisional Application No. 61/048,499, filed Apr. 28, 2008, the entire disclosures of each of which are incorporated by reference herein. U.S. patent application Ser. No. 13/623,017 is a Continuation-In-Part of U.S. patent application Ser. No. 13/223,852, filed Sep. 1, 2011, which is a Continuation of U.S. patent application Ser. No. 11/583,368, now U.S. Pat. No. 8,021,867, filed Oct. 18, 2006, which claims the benefit of priority to U.S. Provisional Application No. 60/727,512, filed Oct. 18, 2005, the entire disclosures of each of which are incorporated by reference herein.

### GOVERNMENT SUPPORT

The invention was supported in part by grants 2R01-GM-0498712, 5F32-GM072322 and 5 DPI OD000122 from the National Institute of General Medical Sciences of National Institutes of Health of the United States of America. Therefore, the U.S. government may have certain rights in the invention.

### REFERENCE TO A SEQUENCE LISTING SUBMITTED AS AN XML FILE VIA PATENT CENTER

The instant application contains a Sequence Listing which has been submitted in ST.26 xml format via Patent Center and is hereby incorporated by reference in its entirety. Said xml copy, created on Jun. 5, 2025, is named “P89339_1310USC9_Seq_List_ST26.xml”, and is 57,420 kilobytes in size.

### FIELD OF THE INVENTION

The invention relates to the field of molecular biology and recombinant nucleic acid technology. In particular, the invention relates to rationally-designed, non-naturally-occurring meganucleases with altered DNA recognition sequence specificity and/or altered affinity. The invention also relates to methods of producing such meganucleases, and methods of producing recombinant nucleic acids and organisms using such meganucleases.

### BACKGROUND OF THE INVENTION

Genome engineering requires the ability to insert, delete, substitute and otherwise manipulate specific genetic sequences within a genome, and has numerous therapeutic and biotechnological applications. The development of effective means for genome modification remains a major goal in gene therapy, agrotechnology, and synthetic biology (Porteus et al. (2005), Nat. Biotechnol. 23:967-73; Tzfira et al. (2005), Trends Biotechnol. 23:567-9; McDaniel et al. (2005), Curr. Opin. Biotechnol. 16:476-83). A common method for inserting or modifying a DNA sequence involves introducing a transgenic DNA sequence flanked by sequences homologous to the genomic target and selecting or screening for a successful homologous recombination event. Recombination with the transgenic DNA occurs rarely but can be stimulated by a double-stranded break in the genomic DNA at the target site. Numerous methods have been employed to create DNA double-stranded breaks, including irradiation and chemical treatments. Although these methods efficiently stimulate recombination, the double-stranded breaks are randomly dispersed in the genome, which can be highly mutagenic and toxic. At present, the inability to target gene modifications to unique sites within a chromosomal background is a major impediment to successful genome engineering.

One approach to achieving this goal is stimulating homologous recombination at a double-stranded break in a target locus using a nuclease with specificity for a sequence that is sufficiently large to be present at only a single site within the genome (see, e.g., Porteus et al. (2005), Nat. Biotechnol. 23:967-73). The effectiveness of this strategy has been demonstrated in a variety of organisms using chimeric fusions between an engineered zinc finger DNA-binding domain and the non-specific nuclease domain of the FokI restriction enzyme (Porteus (2006), Mol Ther 13:438-46; Wright et al. (2005), Plant J. 44:693-705; Urnov et al. (2005), Nature 435:646-51). Although these artificial zinc finger nucleases stimulate site-specific recombination, they retain residual non-specific cleavage activity resulting from under-regulation of the nuclease domain and frequently cleave at unintended sites (Smith et al. (2000), Nucleic Acids Res. 28:3361-9). Such unintended cleavage can cause mutations and toxicity in the treated organism (Porteus et al. (2005), Nat. Biotechnol. 23:967-73).

A group of naturally-occurring nucleases which recognize 15-40 base-pair cleavage sites commonly found in the genomes of plants and fungi may provide a less toxic genome engineering alternative. Such “meganucleases” or “homing endonucleases” are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38:49-95). Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif (see Chevalier et al. (2001), Nucleic Acids Res. 29 (18): 3757-3774). The LAGLIDADG meganucleases with a single copy of the LAGLIDADG motif form homodimers, whereas members with two copies of the LAGLIDADG motif are found as monomers. Similarly, the GIY-YIG family members have a GIY-YIG module, which is 70-100 residues long and includes four or five conserved sequence motifs with four invariant residues, two of which are required for activity (see Van Roey et al. (2002), Nature Struct. Biol. 9:806-811). The His-Cys box meganucleases are characterized by a highly conserved series of histidines and cysteines over a region encompassing several hundred amino acid residues (see Chevalier et al. (2001), Nucleic Acids Res. 29 (18): 3757-3774). In the case of the NHN family, the members are defined by motifs containing two pairs of conserved histidines surrounded by asparagine residues (see Chevalier et al. (2001), Nucleic Acids Res. 29 (18): 3757-3774). The four families of meganucleases are widely separated from one another with respect to conserved structural elements and, consequently, DNA recognition sequence specificity and catalytic activity.

Natural meganucleases, primarily from the LAGLIDADG family, have been used to effectively promote site-specific genome modification in plants, yeast, Drosophila, mammalian cells and mice, but this approach has been limited to the modification of either homologous genes that conserve the meganuclease recognition sequence (Monnat et al. (1999), Biochem. Biophys. Res. Commun. 255:88-93) or to pre-engineered genomes into which a recognition sequence has been introduced (Rouet et al. (1994), Mol. Cell. Biol. 14:8096-106; Chilton et al. (2003), Plant Physiol. 133:956-65; Puchta et al. (1996), Proc. Natl. Acad. Sci. USA 93:5055-60; Rong et al. (2002), Genes Dev. 16:1568-81; Gouble et al. (2006), J. Gene Med. 8 (5): 616-622).

Systematic implementation of nuclease-stimulated gene modification requires the use of engineered enzymes with customized specificities to target DNA breaks to existing sites in a genome and, therefore, there has been great interest in adapting meganucleases to promote gene modifications at medically or biotechnologically relevant sites (Porteus et al. (2005), Nat. Biotechnol. 23:967-73; Sussman et al. (2004), J. Mol. Biol. 342:31-41; Epinat et al. (2003), Nucleic Acids Res. 31:2952-62).

The meganuclease I-CreI from Chlamydomonas reinhardtii is a member of the LAGLIDADG family which recognizes and cuts a 22 base-pair recognition sequence in the chloroplast chromosome, and which presents an attractive target for meganuclease redesign. The wild-type enzyme is a homodimer in which each monomer makes direct contacts with 9 base pairs in the full-length recognition sequence. Genetic selection techniques have been used to identify mutations in I-CreI that alter base preference at a single position in this recognition sequence (Sussman et al. (2004), J. Mol. Biol. 342:31-41; Chames et al. (2005), Nucleic Acids Res. 33: e178; Seligman et al. (2002), Nucleic Acids Res. 30:3870-9) or, more recently, at three positions in the recognition sequence (Arnould et al. (2006), J. Mol. Biol. 355:443-58). The I-CreI protein-DNA interface contains nine amino acids that contact the DNA bases directly and at least an additional five positions that can form potential contacts in modified interfaces. The size of this interface imposes a combinatorial complexity that is unlikely to be sampled adequately in sequence libraries constructed to select for enzymes with drastically altered cleavage sites.

Defects in transcriptional regulation underlie numerous disease states, including cancer. See, e.g., Nebert (2002) Toxicology 181-182:131-41. A major goal of current strategies for correcting such defects is to achieve sufficient specificity of action. See, e.g., Reid et al. (2002) Curr Opin Mol Ther 4:130-137. Designed zinc-finger protein transcription factors (ZFP TFs) emulate natural transcriptional control mechanisms, and therefore provide an attractive tool for precisely regulating gene expression. See, e.g., U.S. Pat. Nos. 6,607,882 and 6,534,261; and Beerli et al. (2000) Proc Natl Acad Sci USA 97:1495-500; Zhang et al. (2000) J Biol Chem 275:33850-60; Snowden et al. (2002) Curr Biol 12:2159-66; Liu et al. (2001) J Biol Chem 276:11323-34; Reynolds et al. (2003) Proc Natl Acad Sci USA 100:1615-20; Bartsevich et al. (2000) Mol. Pharmacol 58:1-10; Ren et al. (2002), Genes Dev 16:27-32; Jamieson et al. (2003), Nat Rev Drug Discov 2:361-368). Accurate control of gene expression is important for understanding gene function (target validation) as well as for developing therapeutics to treat disease. See, e.g., Urnov & Rebar (2002) Biochem Pharmacol 64:919-23.

However, for many disease states, it may be that these proteins, or any other gene regulation technology, will have to be specific for a single gene within the genome, which is a challenging criterion given the size and complexity of the human genome.

Indeed, recent studies with siRNA (Doench et al. (2003), Genes Dev 17:438-42; Jackson et al. (2003), Nat Biotechnol 18:18) and antisense DNA/RNA (Cho et al. (2001), Proc Natl Acad Sci USA 98:9819-23) have fallen far short of obtaining single-gene specificity; illuminating the magnitude of the task of obtaining exogenous regulation of a single specific gene in a genome (e.g., the human genome).

There remains a need for molecules that will facilitate precise targeting of a transcription effector (e.g., an activator or a repressor) to a specific locus in a genome to better regulate endogenous gene expression.

### SUMMARY OF THE INVENTION

The present invention is based, in part, upon the identification and characterization of specific amino acid residues in the LAGLIDADG family of meganucleases that make contacts with DNA bases and the DNA backbone when the meganucleases associate with a double-stranded DNA recognition sequence, and thereby affect the specificity and activity of the enzymes. This discovery has been used, as described in detail below, to identify amino acid substitutions which can alter the recognition sequence specificity and/or DNA-binding affinity of the meganucleases, and to rationally design and develop non-naturally-occurring meganucleases that can recognize a desired DNA sequence that naturally-occurring meganucleases do not recognize. Such non-naturally-occurring, rationally-designed meganucleases can be used in conjunction with regulatory or effector domains to regulate cellular process in vivo and in vitro. In particular, non-naturally occurring, rationally-designed meganucleases can be used in conjunction with a transcription effector domain to provide a targeted transcriptional activator for regulation of gene expression in vivo or in vitro.

In one aspect the invention provides a targeted transcriptional effector comprising: (i) an inactive meganuclease DNA-binding domain that binds to a target recognition site; and (ii) a transcription effector domain, wherein binding of the meganuclease DNA-binding domain targets the transcriptional effector to a gene of interest.

In one embodiment, targeted transcriptional effector further comprises a domain linker joining the meganuclease DNA-binding domain and the transcription effector domain. The domain linker can comprise a polypeptide.

In some embodiments, the meganuclease DNA-binding domain is altered from a naturally-occurring meganuclease by at least one point mutation which reduces or abolishes endonuclease cleavage activity.

The targeted transcriptional effector can further comprise a nuclear localization signal.

In some embodiments, the transcriptional effector domain is a transcription activator or a transcription repressor.

In some embodiments, the meganuclease DNA-binding domain comprises a recombinant meganuclease having altered specificity for at least one recognition sequence half-site relative to a wild-type I-CreI meganuclease, comprising:
a polypeptide having at least 85% sequence similarity to residues 2-153 of the I-CreI meganuclease of SEQ ID NO: 1; and
having specificity for a recognition sequence half-site which differs by at least one base pair from a half-site within an I-CreI meganuclease recognition sequence selected from the group consisting of SEQ ID NO: 2, SEQ ID NO: 3, SEQ ID NO: 4 and SEQ ID NO: 5;
wherein said recombinant meganuclease comprises at least one modification of Table 1 and a modification which reduces or abolishes said endonuclease cleavage activity.

In one embodiment, the modification which reduces or abolishes said endonuclease cleavage activity is Q47E.

In some embodiments, the meganuclease DNA-binding domain comprises a recombinant meganuclease having altered specificity for at least one recognition sequence half-site relative to a wild-type I-MsoI meganuclease, comprising:
a polypeptide having at least 85% sequence similarity to residues 6-160 of the I-MsoI meganuclease of SEQ ID NO: 6; and
having specificity for a recognition sequence half-site which differs by at least one base pair from a half-site within an I-MsoI meganuclease recognition sequence selected from the group consisting of SEQ ID NO: 7 and SEQ ID NO: 8;
wherein said recombinant meganuclease comprises at least one modification of Table 2 and a modification which reduces or abolishes said endonuclease cleavage activity.

In one embodiment, the modification which reduces or abolishes said endonuclease cleavage activity is D22N.

In some embodiments, the meganuclease DNA-binding domain comprises a recombinant meganuclease having altered specificity for a recognition sequence relative to a wild-type I-SceI meganuclease, comprising:
a polypeptide having at least 85% sequence similarity to residues 3-186 of the I-SceI meganuclease of SEQ ID NO: 9; and
having specificity for a recognition sequence which differs by at least one base pair from an I-SceI meganuclease recognition sequence of SEQ ID NO: 10 and SEQ ID NO: 11;
wherein said recombinant meganuclease comprises at least one modification of Table 3 and a modification which reduces or abolishes said endonuclease cleavage activity.

In one embodiment, the modification which reduces or abolishes said endonuclease cleavage activity is D44N or D145N.

In some embodiments, the meganuclease DNA-binding domain comprises a recombinant meganuclease having altered specificity for at least one recognition sequence half-site relative to a wild-type I-CeuI meganuclease, comprising:
a polypeptide having at least 85% sequence similarity to residues 5-211 of the I-CeuI meganuclease of SEQ ID NO: 12; and
having specificity for a recognition sequence half-site which differs by at least one base pair from a half-site within an I-CeuI meganuclease recognition sequence selected from the group consisting of SEQ ID NO: 13 and SEQ ID NO: 14;
wherein said recombinant meganuclease comprises at least one modification of Table 4 and a modification which reduces said endonuclease cleavage activity.

In one embodiment, the modification which reduces said endonuclease cleavage activity is E66Q.

In some embodiments, the meganuclease DNA-binding domain comprises a recombinant meganuclease having altered specificity for at least one recognition sequence half-site relative to a wild-type I-CreI meganuclease, comprising:
a polypeptide having at least 85% sequence similarity to residues 2-153 of the I-CreI meganuclease of SEQ ID NO: 1; and
having specificity for a recognition sequence half-site which differs by at least one base pair from a half-site within an I-CreI meganuclease recognition sequence selected from the group consisting of SEQ ID NO: 2, SEQ ID NO: 3, SEQ ID NO: 4 and SEQ ID NO: 5;
wherein:
(1) specificity at position −1 has been altered:
   (a) to a T on a sense strand by a modification selected from the group consisting of Q70, C70, L70, Y75, Q75, H75, H139, Q46 and H46;
   (b) to an A on a sense strand by a modification selected from the group consisting of Y75, L75, C75, Y139, C46 and A46;
   (c) to a G on a sense strand by a modification selected from the group consisting of K70, E70, E75, E46 and D46;
   (d) to a C on a sense strand by a modification selected from the group consisting of H75, R75, H46, K46 and R46; or
   (e) to any base on a sense strand by a modification selected from the group consisting of G70, A70, S70 and G46; and/or
(2) specificity at position −2 has been altered:
   (a) to an A on a sense strand by a modification selected from the group consisting of Q70, T44, A44, V44, I44, L44, and N44;
   (b) to a C on a sense strand by a modification selected from the group consisting of E70, D70, K44 and R44;
   (c) to a G on a sense strand by a modification selected from the group consisting of H70, D44 and E44; or
   (d) to an A or T on a sense strand by a modification comprising C44; and/or
(3) specificity at position −3 has been altered:
   (a) to an A on a sense strand by a modification selected from the group consisting of Q68 and C24;
   (b) to a C on a sense strand by a modification selected from the group consisting of E68, F68, K24 and R24;
   (c) to a T on a sense strand by a modification selected from the group consisting of M68, C68, L68 and F68;
   (d) to an A or C on a sense strand by a modification comprising H68;
   (e) to a C or T on a sense strand by a modification comprising Y68; or
   (f) to a G or T on a sense strand by a modification comprising K68; and/or
(4) specificity at position −4 has been altered:
   (a) to a C on a sense strand by a modification selected from the group consisting of E77 and K26;
   (b) to a G on a sense strand by a modification selected from the group consisting of E26 and R77;
   (c) to a C or T on a sense strand by a modification comprising S77; or
   (d) to a any base on a sense strand by a modification comprising S26; and/or
(5) specificity at position −5 has been altered:
   (a) to a C on a sense strand by a modification comprising E42;
   (b) to a G on a sense strand by a modification comprising R42;
   (c) to an A or G on a sense strand by a modification selected from the group consisting of C28 and Q42; or
   (d) to any base on a sense strand by a modification of selected from the group consisting of M66 and K66; and/or
(6) specificity at position −6 has been altered:
   (a) to a T on a sense strand by a modification selected from the group consisting of C40, I40, V40, C79, I79, V79, and Q28;
   (b) to a C on a sense strand by a modification selected from the group consisting of E40 and R28; or
   (c) to a G on a sense strand by a modification comprising R40; and/or
(7) specificity at position −7 has been altered:
   (a) to a C on a sense strand by a modification selected from the group consisting of E38, K30 and R30;
   (b) to a G on a sense strand by a modification selected from the group consisting of K38, R38 and E30;
   (c) to a T on a sense strand by a modification selected from the group consisting of I38 and L38; or
   (d) to an A or G on a sense strand by a modification comprising C38; or
   (e) to any base on a sense strand by a modification selected from the group consisting of H38, N38 and Q30; and/or
(8) specificity at position −8 has been altered:
   (a) to a T on a sense strand by a modification selected from the group consisting of L33, V33, I33, F33 and C33;
   (b) to a C on a sense strand by a modification selected from the group consisting of E33 and D33;
   (c) to a G on a sense strand by a modification consisting of K33;
   (d) to an A or C on a sense strand by a modification comprising R32; or
   (e) to an A or G on a sense strand by a modification comprising R33; and/or
(9) specificity at position −9 has been altered:
   (a) to a C on a sense strand by a modification comprising E32;
   (b) to a G on a sense strand by a modification selected from the group consisting of R32 and K32;
   (c) to a T on a sense strand by a modification selected from the group consisting of L32, V32, A32 and C32;
   (d) to a C or T on a sense strand by a modification selected from the group consisting of D32 and I32; or
   (e) to any base on a sense strand by a modification selected from the group consisting of S32, N32, H32, Q32 and T32.

In some embodiments, the meganuclease DNA-binding domain comprises a recombinant meganuclease having altered specificity for at least one recognition sequence half-site relative to a wild-type I-MsoI meganuclease, comprising:
a polypeptide having at least 85% sequence similarity to residues 6-160 of the I-MsoI meganuclease of SEQ ID NO: 6; and
having specificity for a recognition sequence half-site which differs by at least one base pair from a half-site within an I-MsoI meganuclease recognition sequence selected from the group consisting of SEQ ID NO: 7 and SEQ ID NO: 8;
wherein:
(1) specificity at position −1 has been altered:
   (a) to an A on a sense strand by a modification selected from the group consisting of K75, Q77, A49, C49 and K79;
   (b) to a T on a sense strand by a modification selected from the group consisting of C77, L77 and Q79; or
   (c) to a G on a sense strand by a modification selected from the group consisting of K77, R77, E49 and E79; and/or
(2) specificity at position −2 has been altered:
   (a) to an A on a sense strand by a modification selected from the group consisting of Q75, K81, C47, I47 and L47;
   (b) to a C on a sense strand by a modification selected from the group consisting of E75, D75, R47, K47, K81 and R81; or
   (c) to a G on a sense strand by a modification selected from the group consisting of K75, E47 and E81; and/or
(3) specificity at position −3 has been altered:
   (a) to an A on a sense strand by a modification selected from the group consisting of Q72, C26, L26, V26, A26 and I26;
   (b) to a C on a sense strand by a modification selected from the group consisting of E72, Y72, H26, K26 and R26; or
   (c) to a T on a sense strand by a modification selected from the group consisting of K72, Y72 and H26; and/or
(4) specificity at position −4 has been altered:
   (a) to a T on a sense strand by a modification selected from the group consisting of K28, K83 and Q28;
   (b) to a G on a sense strand by a modification selected from the group consisting of R83 and K83; or
   (c) to an A on a sense strand by a modification selected from the group consisting of K28 and Q83; and/or
(5) specificity at position −5 has been altered:
   (a) to a G on a sense strand by a modification selected from the group consisting of R45 and E28;
   (b) to a T on a sense strand by a modification comprising Q28; or
   (c) to a C on a sense strand by a modification comprising R28; and/or
(6) specificity at position −6 has been altered:
   (a) to a T on a sense strand by a modification selected from the group consisting of K43, V85, L85 and Q30;
   (b) to a C on a sense strand by a modification selected from the group consisting of E43, E85, K30 and R30; or
   (c) to a G on a sense strand by a modification selected from the group consisting of R43, K43, K85, R85, E30 and D30; and/or
(7) specificity at position −7 has been altered:
   (a) to a C on a sense strand by a modification selected from the group consisting of E32 and E41;
   (b) to a G on a sense strand by a modification selected from the group consisting of R32, R41 and K41;
   (c) to a T on a sense strand by a modification selected from the group consisting of K32, M41, L41 and 141; and/or
(8) specificity at position −8 has been altered:
   (a) to a T on a sense strand by a modification selected from the group consisting of K32 and K35;
   (b) to a C on a sense strand by a modification comprising E32; or
   (c) to a G on a sense strand by a modification consisting of K32, K35 and R35; and/or
(9) specificity at position −9 has been altered:
   (a) to an A on a sense strand by a modification selected from the group consisting of N34 and H34;
   (b) to a T on a sense strand by a modification selected from the group consisting of S34, C34, V34, T34 and A34; or
   (c) to a G on a sense strand by a modification selected from the group consisting of K34, R34 and H34.

In some embodiments, the meganuclease DNA-binding domain comprises recombinant meganuclease having altered specificity for a recognition sequence relative to a wild-type I-SceI meganuclease, comprising:
a polypeptide having at least 85% sequence similarity to residues 3-186 of the I-SceI meganuclease of SEQ ID NO: 9; and
having specificity for a recognition sequence which differs by at least one base pair from an I-SceI meganuclease recognition sequence of SEQ ID NO: 10 and SEQ ID NO: 11;
wherein:
(1) specificity at position 4 has been altered:
   (a) to an A on a sense strand by a modification comprising K50;
   (b) to a T on a sense strand by a modification selected from the group consisting of K57, M57 and Q50; or
   (c) to a G on a sense strand by a modification selected from the group consisting of E50, R57 and K57; and/or
(2) specificity at position 5 has been altered:
   (a) to an A on a sense strand by a modification selected from the group consisting of K48, Q102;
   (b) to a G on a sense strand by a modification selected from the group consisting of E48, K102 and R102; or
   (c) to a T on a sense strand by a modification selected from the group consisting of Q48, C102, L102 and V102; and/or
(3) specificity at position 6 has been altered:
   (a) to an A on a sense strand by a modification comprising K59;
   (b) to a C on a sense strand by a modification selected from the group consisting of R59 and K59; or
   (b) to a G on a sense strand by a modification selected from the group consisting of K84 and E59; and/or
(4) specificity at position 7 has been altered:
   (a) to a C on a sense strand by a modification selected from the group consisting of R46, K46 and E86;
   (b) to a G on a sense strand by a modification selected from the group consisting of K86, R86 and E46; or
   (c) to an A on a sense strand by a modification selected from the group consisting of C46, L46 and V46; and/or
(5) specificity at position 8 has been altered:
   (a) to a C on a sense strand by a modification selected from the group consisting of E88, R61 and H61;
   (b) to a T on a sense strand by a modification selected from the group consisting of K88, Q61 and H61; or
   (c) to an A on a sense strand by a modification selected from the group consisting of K61, S61, V61, A61 and L61; and/or
(6) specificity at position 9 has been altered:
   (a) to an A on a sense strand by a modification selected from the group consisting of C98, V98 and L98;
   (b) to a C on a sense strand by a modification selected from the group consisting of R98 and K98; or
   (c) to a G on a sense strand by a modification selected from the group consisting of E98 and D98; and/or
(7) specificity at position 10 has been altered:
   (a) to a C on a sense strand by a modification selected from the group consisting of K96 and R96;
   (b) to a G on a sense strand by a modification selected from the group consisting of D96 and E96; or
   (c) to an A on a sense strand by a modification selected from the group consisting of C96 and A96; and/or
(8) specificity at position 11 has been altered:
   (a) to a T on a sense strand by a modification comprising Q90;
   (b) to a C on a sense strand by a modification selected from the group consisting of K90 and R90; or
   (c) to a G on a sense strand by a modification comprising E90; and/or
(9) specificity at position 12 has been altered:
   (a) to an A on a sense strand by a modification comprising Q193;
   (b) to a C on a sense strand by a modification selected from the group consisting of E165, E193 and D193; or
   (c) to a G on a sense strand by a modification selected from the group consisting of K165 and R165; and/or
(10) specificity at position 13 has been altered:
   (a) to a T on a sense strand by a modification selected from the group consisting of Q193, C163 and L163;
   (b) to a G on a sense strand by a modification selected from the group consisting of E193, D193, K163 and R192; or
   (c) to an A on a sense strand by a modification selected from the group consisting of C193 and L193; and/or
(11) specificity at position 14 has been altered:
   (a) to a T on a sense strand by a modification selected from the group consisting of K161 and Q192;
   (b) to an A on a sense strand by a modification selected from the group consisting of L192 and C192;
   (c) to a G on a sense strand by a modification selected from the group consisting of K147, K161, R161, R197, D192 and E192; or
   (d) to a T on a sense strand by a modification selected from the group consisting of K161 and Q192; and/or
(12) specificity at position 15 has been altered:
   (a) to a T on a sense strand by a modification selected from the group consisting of C151, L151 and K151;
   (b) to a G on a sense strand by a modification comprising K151; or
   (c) to a C on a sense strand by a modification comprising E151; and/or
(13) specificity at position 17 has been altered:
   (a) to a T on a sense strand by a modification selected from the group consisting of G152 and Q150;
   (b) to a C on a sense strand by a modification selected from the group consisting of K152 and K150; or
   (c) to a G on a sense strand by a modification selected from the group consisting of N152, S152, D152, D150 and E150; and/or
(14) specificity at position 18 has been altered:
   (a) to a T on a sense strand by a modification selected from the group consisting of H155 and Y155;
   (b) to a C on a sense strand by a modification selected from the group consisting of R155 and K155; or
   (c) to an A on a sense strand by a modification selected from the group consisting of K155 and C155.

In some embodiments, the meganuclease DNA-binding domain comprises a recombinant meganuclease having altered specificity for at least one recognition sequence half-site relative to a wild-type I-CeuI meganuclease, comprising:
a polypeptide having at least 85% sequence similarity to residues 5-211 of the I-CeuI meganuclease of SEQ ID NO: 12; and
having specificity for a recognition sequence half-site which differs by at least one base pair from a half-site within an I-CeuI meganuclease recognition sequence selected from the group consisting of SEQ ID NO: 13 and SEQ ID NO: 14;
wherein:
(1) specificity at position −1 has been altered:
   (a) to an A on a sense strand by a modification selected from the group consisting of C92, A92 and V92;
   (b) to a T on a sense strand by a modification selected from the group consisting of Q116 and Q92; or
   (c) to a G on a sense strand by a modification selected from the group consisting of E116 and E92; and/or
(2) specificity at position −2 has been altered:
   (a) to an A on a sense strand by a modification selected from the group consisting of Q117, C90, L90 and V90;
   (b) to a G on a sense strand by a modification selected from the group consisting of K117, R124, K124, E124, E90 and D90; or
   (c) to a C on a sense strand by a modification selected from the group consisting of E117, D117, R174, K124, K90, R90 and K68; and/or
(3) specificity at position −3 has been altered:
   (a) to an A on a sense strand by a modification selected from the group consisting of C70, V70, T70, L70 and K70;
   (b) to a T on a sense strand by a modification comprising Q70;
   (b) to a C on a sense strand by a modification consisting of K70; and/or
(4) specificity at position −4 has been altered:
   (a) to a C on a sense strand by a modification selected from the group consisting of E126, D126, R88, K88 and K72;
   (b) to a T on a sense strand by a modification selected from the group consisting of K126, L126 and Q88; or
   (c) to an A on a sense strand by a modification selected from the group consisting of Q126, N126, K88, L88, C88, C72, L72 and V72; and/or
(5) specificity at position −5 has been altered:
   (a) to a G on a sense strand by a modification selected from the group consisting of E74, K128, R128 and E128;
   (b) to a T on a sense strand by a modification selected from the group consisting of C128, L128, V128 and T128; or
   (c) to an A on a sense strand by a modification selected from the group consisting of C74, L74, V74 and T74; and/or
(6) specificity at position −6 has been altered:
   (a) to a T on a sense strand by a modification selected from the group consisting of K86, C86 and L86;
   (b) to a C on a sense strand by a modification selected from the group consisting of D86, E86, R84 and K84; or
   (c) to a G on a sense strand by a modification selected from the group consisting of K128, R128, R86, K86 and E84; and/or
(7) specificity at position −7 has been altered:
   (a) to a C on a sense strand by a modification selected from the group consisting of R76, K76 and H76;
   (b) to a G on a sense strand by a modification selected from the group consisting of E76 and R84; or
   (c) to a T on a sense strand by a modification consisting of H76 and Q76; and/or
(8) specificity at position −8 has been altered:
   (a) to an A on a sense strand by a modification selected from the group consisting of Y79, R79 and Q76;
   (b) to a C on a sense strand by a modification selected from the group consisting of D79, E79, D76 and E76; or
   (c) to a G on a sense strand by a modification selected from the group consisting of R79, K79, K76 and R76; and/or
(9) specificity at position −9 has been altered:
   (a) to a T on a sense strand by a modification selected from the group consisting of K78, V78, L78, C78 and T78;
   (b) to a C on a sense strand by a modification selected from the group consisting of D78 and E78; or
   (c) to a G on a sense strand by a modification selected from the group consisting of R78, K78 and H78.

In one embodiment, the meganuclease DNA-binding domain comprises a recombinant meganuclease having altered binding affinity for double-stranded DNA relative to a wild-type I-CreI meganuclease, comprising:
a polypeptide having at least 85% sequence similarity to residues 2-153 of the I-CreI meganuclease of SEQ ID NO: 1;
wherein DNA-binding affinity has been increased by at least one modification corresponding to:
(a) substitution of E80, D137, I81, L112, P29, V64 or Y66 with H, N, Q, S, T, K or R; or
(b) substitution of T46, T140 or T143 with K or R.

In another embodiment, the meganuclease DNA-binding domain comprises a recombinant meganuclease having altered binding affinity for double-stranded DNA relative to a wild-type I-CreI meganuclease, comprising:
a polypeptide having at least 85% sequence similarity to residues 2-153 of the I-CreI meganuclease of SEQ ID NO: 1;
wherein DNA-binding affinity has been decreased by at least one modification corresponding to:
(a) substitution of K34, K48, R51, K82, K116 or K139 with H, N, Q, S, T, D or E; or
(b) substitution of 181, L112, P29, V64, Y66, T46, T140 or T143 with D or E.

In one embodiment, the meganuclease DNA-binding domain comprises a recombinant meganuclease having altered binding affinity for double-stranded DNA relative to a wild-type I-MsoI meganuclease, comprising:
a polypeptide having at least 85% sequence similarity to residues 6-160 of the I-MsoI meganuclease of SEQ ID NO: 6;
wherein DNA-binding affinity has been increased by at least one modification corresponding to:
(a) substitution of E147, I85, G86 or Y118 with H, N, Q, S, T, K or R; or
(b) substitution of Q41, N70, S87, T88, H89, Q122, Q139, S150 or N152 with K or R.

In another embodiment, the meganuclease DNA-binding domain comprises a recombinant meganuclease having altered binding affinity for double-stranded DNA relative to a wild-type I-MsoI meganuclease, comprising:
a polypeptide having at least 85% sequence similarity to residues 6-160 of the I-MsoI meganuclease of SEQ ID NO: 6;
wherein DNA-binding affinity has been decreased by at least one modification corresponding to:
(a) substitution of K36, R51, K123, K143 or R144 with H, N, Q, S, T, D or E; or
(b) substitution of 185, G86, Y118, Q41, N70, S87, T88, H89, Q122, Q139, S150 or N152 with D or E.

In one embodiment, the meganuclease DNA-binding domain comprises a recombinant meganuclease having altered binding affinity for double-stranded DNA relative to a wild-type I-SceI meganuclease, comprising:
a polypeptide having at least 85% sequence similarity to residues 3-186 of the I-SceI meganuclease of SEQ ID NO: 9;
wherein DNA-binding affinity has been increased by at least one modification corresponding to:
(a) substitution of D201, L19, L80, L92, Y151, Y188, I191, Y199 or Y222 with H, N, Q, S, T, K or R; or
(b) substitution of N15, N17, S81, H84, N94, N120, T156, N157, S159, N163, Q165, S166, N194 or S202 with K or R.

In another embodiment, the meganuclease DNA-binding domain comprises a recombinant meganuclease having altered binding affinity for double-stranded DNA relative to a wild-type I-SceI meganuclease, comprising:
a polypeptide having at least 85% sequence similarity to residues 3-186 of the I-SceI meganuclease of SEQ ID NO: 9;
wherein DNA-binding affinity has been decreased by at least one modification corresponding to:
(a) substitution of K20, K23, K63, K122, K148, K153, K190, K193, K195 or K223 with H, N, Q, S, T, D or E; or
(b) substitution of L19, L80, L92, Y151, Y188, I191, Y199, Y222, N15, N17, S81, H84, N94, N120, T156, N157, S159, N163, Q165, S166, N194 or S202 with D or E.

In one embodiment, meganuclease DNA-binding domain comprises a recombinant meganuclease having altered binding affinity for double-stranded DNA relative to a wild-type I-CeuI meganuclease, comprising:
a polypeptide having at least 85% sequence similarity to residues 5-211 of the I-CeuI meganuclease of SEQ ID NO: 12;
wherein DNA-binding affinity has been increased by at least one modification corresponding to:
(a) substitution of D25 or D128 with H, N, Q, S, T, K or R; or
(b) substitution of S68, N70, H94, S117, N120, N129 or H172 with K or R.

In another embodiment, the meganuclease DNA-binding domain comprises a recombinant meganuclease having altered binding affinity for double-stranded DNA relative to a wild-type I-CeuI meganuclease, comprising:
a polypeptide having at least 85% sequence similarity to residues 5-211 of the I-CeuI meganuclease of SEQ ID NO: 12;
wherein DNA-binding affinity has been decreased by at least one modification corresponding to:
(a) substitution of K21, K28, K31, R112, R114 or R130 with H, N, Q, S, T, D or E; or
(b) substitution of S68, N70, H94, S117, N120, N129 or H172 with D or E.

In one embodiment, the meganuclease DNA-binding domain comprises a recombinant meganuclease monomer having altered affinity for dimer formation with a reference meganuclease monomer, comprising:
a polypeptide having at least 85% sequence similarity to residues 2-153 of the I-CreI meganuclease of SEQ ID NO: 1;
wherein affinity for dimer formation has been altered by at least one modification corresponding to:
(a) substitution of K7, K57 or K96 with D or E; or
(b) substitution of E8 or E61 with K or R.

In another embodiment, the meganuclease DNA-binding domain comprises a recombinant meganuclease heterodimer comprising:
a first polypeptide having at least 85% sequence similarity to residues 2-153 of the I-CreI meganuclease of SEQ ID NO: 1;
wherein affinity for dimer formation has been altered by at least one modification corresponding to substitution of K7, K57 or K96 with D or E; and
a second polypeptide having at least 85% sequence similarity to residues 2-153 of the I-CreI meganuclease of SEQ ID NO: 1;
wherein affinity for dimer formation has been altered by at least one modification corresponding to a substitution of E8 or E61 with K or R.

In one embodiment, the meganuclease DNA-binding domain comprises a recombinant meganuclease monomer having altered affinity for dimer formation with a reference meganuclease monomer, comprising:
a polypeptide having at least 85% sequence similarity to residues 6-160 of the I-MsoI meganuclease of SEQ ID NO: 6;
wherein affinity for dimer formation has been altered by at least one modification corresponding to:
(a) substitution of R302 with D or E; or
(b) substitution of D20, E11 or Q64 with K or R.

In another embodiment, the meganuclease DNA-binding domain comprises a recombinant meganuclease heterodimer comprising:
a first polypeptide having at least 85% sequence similarity to residues 6-160 of the I-MsoI meganuclease of SEQ ID NO: 6;
wherein affinity for dimer formation has been altered by at least one modification corresponding to a substitution of R302 with D or E; and
a second polypeptide having at least 85% sequence similarity to residues 6-160 of the I-MsoI meganuclease of SEQ ID NO: 6;
wherein affinity for dimer formation has been altered by at least one modification corresponding to a substitution of D20, E11 or Q64 with K or R.

In one embodiment, the meganuclease DNA-binding domain comprises a recombinant meganuclease monomer having altered affinity for dimer formation with a reference meganuclease monomer, comprising:
a polypeptide having at least 85% sequence similarity to residues 5-211 of the I-CeuI meganuclease of SEQ ID NO: 12;
wherein affinity for dimer formation has been altered by at least one modification corresponding to:
(a) substitution of R93 with D or E; or
(b) substitution of E152 with K or R.

In another embodiment, meganuclease DNA-binding domain comprises a recombinant meganuclease heterodimer comprising:
a first polypeptide having at least 85% sequence similarity to residues 5-211 of the I-CeuI meganuclease of SEQ ID NO: 12;
wherein affinity for dimer formation has been altered by at least one modification corresponding to a substitution of R93 with D or E; and
a second polypeptide having at least 85% sequence similarity to residues 5-211 of the I-CeuI meganuclease of SEQ ID NO: 12;
wherein affinity for dimer formation has been altered by at least one modification corresponding to a substitution of E152 with K or R.

In some embodiments, the recombinant meganuclease monomer or heterodimer further comprises at least one modification selected from Table 1.

In another aspect, the invention provides a nucleic acid encoding the targeted transcriptional effector.

In yet another aspect, the invention provides a method for treating a disease or condition in a subject in need thereof, the method comprising: introducing the nucleic acid encoding the targeted transcriptional effector into a subject, whereby the polypeptide encoded by the nucleic acid binds to the target site and affects transcription of the gene of interest.

In still another aspect, the invention provides a method for treating a disease or condition in a subject in need thereof, the method comprising: introducing the targeted transcriptional effector of claims 1-34 into a subject, whereby the polypeptide binds to the target site and affects transcription of the gene of interest.

These and other aspects and embodiments of the invention will be apparent to one of ordinary skill in the art based upon the following detailed description of the invention.

### BRIEF DESCRIPTION OF THE FIGURES

FIG. 1A illustrates the interactions between the I-CreI homodimer and its naturally-occurring double-stranded recognition sequence, based upon crystallographic data. This schematic representation depicts the recognition sequence (SEQ ID NO: 2 and SEQ ID NO: 3), shown as unwound for illustration purposes only, bound by the homodimer, shown as two ovals. The bases of each DNA half-site are numbered −1 through −9, and the amino acid residues of I-CreI which form the recognition surface are indicated by one-letter amino acid designations and numbers indicating residue position. Solid black lines: hydrogen bonds to DNA bases. Dashed lines: amino acid positions that form additional contacts in enzyme designs but do not contact the DNA in the wild-type complex. Arrows: residues that interact with the DNA backbone and influence cleavage activity.

FIG. 1B illustrates the wild-type contacts between the A-T base pair at position −4 of the cleavage half-site on the right side of FIG. 1A. Specifically, the residue Q26 is shown to interact with the A base. Residue I77 is in proximity to the base pair but not specifically interacting.

FIG. 1C illustrates the interactions between a non-naturally-occurring, rationally-designed variant of the I-CreI meganuclease in which residue I77 has been modified to E77. As a result of this change, a G-C base pair is preferred at position −4. The interaction between Q26 and the G base is mediated by a water molecule, as has been observed crystallographically for the cleavage half-site on the left side of FIG. 1A.

FIG. 1D illustrates the interactions between a non-naturally-occurring, rationally-designed variant of the I-CreI meganuclease in which residue Q26 has been modified to E26 and residue I77 has been modified to R77. As a result of this change, a C-G base pair is preferred at position −4.

FIG. 1E illustrates the interactions between a non-naturally-occurring, rationally-designed variant of the I-CreI meganuclease in which residue Q26 has been modified to A26 and residue I77 has been modified to Q77. As a result of this change, a T-A base pair is preferred at position −4.

FIG. 2A shows a comparison of one recognition sequence for each of the wild type I-CreI meganuclease (WT) and 11 non-naturally-occurring, rationally-designed meganuclease heterodimers described herein. Bases that are conserved relative to the WT recognition sequence are shaded. The 9 bp half-sites are bolded. WT: wild-type (SEQ ID NO: 4); CF: ΔF508 allele of the human CFTR gene responsible for most cases of cystic fibrosis (SEQ ID NO: 25); MYD: the human DM kinase gene associated with myotonic dystrophy (SEQ ID NO: 27); CCR: the human CCR5 gene (a major HIV co-receptor) (SEQ ID NO: 26); ACH: the human FGFR3 gene correlated with achondroplasia (SEQ ID NO: 23); TAT: the HIV-1 TAT/REV gene (SEQ ID NO: 15); HSV: the HSV-1 UL36 gene (SEQ ID NO: 28); LAM: the bacteriophage λ p05 gene (SEQ ID NO: 22); POX: the Variola (smallpox) virus gp009 gene (SEQ ID NO: 30); URA: the Saccharomyces cerevisiae URA3 gene (SEQ ID NO: 36); GLA: the Arabidopsis thaliana GL2 gene (SEQ ID NO: 32); BRP: the Arabidopsis thaliana BP-1 gene (SEQ ID NO: 33).

FIG. 2B illustrates the results of incubation of each of wild-type I-CreI (WT) and 11 non-naturally-occurring, rationally-designed meganuclease heterodimers with plasmids harboring the recognition sites for all 12 enzymes for 6 hours at 37° C. Percent cleavage is indicated in each box.

FIGS. 3A and 3B illustrates cleavage patterns of wild-type and non-naturally-occurring, rationally-designed I-CreI homodimers. (FIG. 3A) wild type I-CreI. (FIG. 3B) I-CreI K116D. (C-L) non-naturally-occurring, rationally-designed meganucleases described herein. Enzymes were incubated with a set of plasmids harboring palindromes of the intended cleavage half-site the 27 corresponding single-base pair variations. Bar graphs show fractional cleavage (F) in 4 hours at 37° C. Black bars: expected cleavage patterns based on Table 1. Gray bars: DNA sites that deviate from expected cleavage patterns. White squares indicate bases in the intended recognition site. Also shown are cleavage time-courses over two hours. The open circle time-course plots in C and L correspond to cleavage by the CCR1 and BRP2 enzymes lacking the E80Q mutation. The cleavage sites correspond to the 5′ (left column) and 3′ (right column) half-sites for the heterodimeric enzymes described in FIG. 2A.

FIG. 4 demonstrates DNA recognition by Endo-TNF. Purified Endo-TNF_{SC} was incubated with pUC-19 plasmid substrates (linearized with ScaI) for 2 hours at 37° C. Lanes 1 and 2: molecular weight markers. Lanes 3 and 4: Endo-TNF_{SC} incubated with empty plasmid (lane 3) or plasmid harboring the wild-type I-CreI site (lane 4). Lanes 5-7: linearized plasmid harboring the Endo-TNF_{SC} recognition site incubated with buffer only (lane 5), Endo-TNF_{SC} (lane 6), or the inactivated Endo-TNF_{KO}. Bands of 0.9 and 1.8 kb in length in lane 6 indicate cleavage by Endo-TNF_{SC} of its intended recognition site.

FIG. 5 shows the results of a chromatin immunoprecipitation (ChIP) assay with Endo-TNF_{KO}. Cultured HEK 293 cells were transfected with either GFP or Endo-TNF_{KO} and a ChIP assay was performed. PCR was performed on DNA isolated from input cell lysates (In) or on DNA isolated from cell lysates immunoprecipitated with I-CreI antiserum (IP) or fetal bovine serum (−AB) using primers specific for TNF-α.

FIGS. 6A to 6B demonstrate activity of the CCR2_{REP} transcription repressor. FIG. 6A Schematic of the transcription reporter used in these experiments. An E. coli Lac-Z gene is driven by a 5′-truncated CMV promoter with a CCR2_{REP} recognition sequence at its 5′ end. FIG. 6B A plasmid carrying the reporter expression cassette in (FIG. 6A) was used to transfect cultured HEK 293 cells 24 hours following transfection with a plasmid carrying the CCR2_{REP} gene under the control of a CMV promoter or an empty pCI plasmid (no CCR2_{REP}). Alternatively, cells were transfected with a GFP expression plasmid to normalize for transfection efficiency (GFP). 24 hours post-transfection, cells were harvested and assayed for Lac-Z activity. It was found that cells transfected with the CCR2_{REP} expression plasmid yielded a ˜2.6-fold reduction in Lac-Z activity relative to the mock-transfected control.

### DETAILED DESCRIPTION OF THE INVENTION

### 1.1 Introduction

The present invention is based, in part, upon the identification and characterization of specific amino acids in the LAGLIDADG family of meganucleases that make specific contacts with DNA bases and non-specific contacts with the DNA backbone when the meganucleases associate with a double-stranded DNA recognition sequence, and which thereby affect the recognition sequence specificity and DNA-binding affinity of the enzymes. This discovery has been used, as described in detail below, to identify amino acid substitutions in the meganucleases that can alter the specificity and/or affinity of the enzymes, and to rationally design and develop non-naturally-occurring meganucleases that can recognize a desired DNA sequence that naturally-occurring meganucleases do not recognize, and/or that have increased or decreased specificity and/or affinity relative to the naturally-occurring meganucleases. In addition, the invention provides non-naturally-occurring, rationally-designed meganucleases in which residues at the interface between the monomers associated to form a dimer have been modified in order to promote heterodimer formation. Finally, specific residues have been identified which can be altered to reduce or eliminate the catalytic activity of the meganucleases without destroying the sequence-specific DNA-binding ability. Thus, these altered non-naturally-occurring, rationally-designed meganucleases can be used as DNA-binding proteins to target effector domains to desired loci in a genome.

As a general matter, the invention provides methods for generating non-naturally-occurring, rationally-designed LAGLIDADG meganucleases containing altered amino acid residues at sites within the meganuclease that are responsible for (1) sequence-specific binding to individual bases in the double-stranded DNA recognition sequence, or (2) non-sequence-specific binding to the phosphodiester backbone of a double-stranded DNA molecule. Altering the amino acids involved in binding to the DNA backbone can alter not only the activity of the enzyme, but also the degree of specificity or degeneracy of binding to the recognition sequence by increasing or decreasing overall binding affinity for the double-stranded DNA. Finally, specific residues can be altered to reduce or eliminate catalytic activity. These altered non-naturally-occurring, rationally-designed meganucleases can be used as DNA-binding proteins to target effector domains to desired loci in a genome.

As described in detail below, the methods of rationally-designing non-naturally-occurring meganucleases include the identification of the amino acids responsible for DNA recognition/binding, and the application of a series of rules for selecting appropriate amino acid changes. With respect to meganuclease sequence specificity, the rules include both steric considerations relating to the distances in a meganuclease-DNA complex between the amino acid side chains of the meganuclease and the bases in the sense and anti-sense strands of the DNA, and considerations relating to the non-covalent chemical interactions between functional groups of the amino acid side chains and the desired DNA base at the relevant position.

Finally, a majority of natural meganucleases that bind DNA as homodimers recognize pseudo- or completely palindromic recognition sequences. Because lengthy palindromes are expected to be rare, the likelihood of encountering a palindromic sequence at a genomic site of interest is exceedingly low. Consequently, if these enzymes are to be redesigned to recognize genomic sites of interest, it is necessary to design two enzyme monomers recognizing different half-sites that can heterodimerize to cleave the non-palindromic hybrid recognition sequence. Therefore, in some aspects, the invention provides non-naturally-occurring, rationally-designed meganucleases in which monomers differing by at least one amino acid position are dimerized to form heterodimers. In some cases, both monomers are rationally-designed to form a heterodimer which recognizes a non-palindromic recognition sequence. A mixture of two different monomers can result in up to three active forms of meganuclease dimer: the two homodimers and the heterodimer. In addition or alternatively, in some cases, amino acid residues are altered at the interfaces at which monomers can interact to form dimers, in order to increase or decrease the likelihood of formation of homodimers or heterodimers. In addition or alternatively, in some cases, a linker such as a polypeptide is added between the monomer domains to aid in heterodimer formation.

Thus, in one aspect, the invention provide methods for rationally designing non-naturally-occurring LAGLIDADG meganucleases containing amino acid changes that alter the specificity and/or affinity of the enzymes for DNA-binding. In another aspect, the invention provides the non-naturally-occurring, rationally-designed meganucleases resulting from these methods and their use as sequence-specific DNA-binding proteins to target effector domains to specific loci in a genome. In another aspect, the invention provides methods that use such fusion molecules of non-naturally-occurring, rationally-designed meganucleases and effector domains to regulate gene expression in vivo or in vitro. In another aspect, the invention provides methods for treating conditions which can be treated by increasing or decreasing the expression of a gene, by administering a fusion molecule provided by the invention.

### 1.2 References and Definitions

The patent and scientific literature referred to herein establishes knowledge that is available to those of skill in the art. The issued U.S. patents, patent applications, published foreign applications, and references, including GenBank database sequences, that are cited herein are hereby incorporated by reference to the same extent as if each was specifically and individually indicated to be incorporated by reference.

As used herein, the term “meganuclease” refers to an endonuclease that binds double-stranded DNA at a recognition sequence that is greater than 12 base pairs. Naturally-occurring meganucleases can be monomeric (e.g., I-SceI) or dimeric (e.g., I-CreI). The term meganuclease, as used herein, can be used to refer to monomeric meganucleases, dimeric meganucleases, or to the monomers which associate to form a dimeric meganuclease. The term “homing endonuclease” is synonymous with the term “meganuclease.” The meganucleases can be catalytically active (i.e., capable of binding and cleaving double-stranded DNA at their recognition sequence) or can be inactivated by way of rational design. For most embodiments described herein, the meganuclease will be inactivated, although catalytically active meganucleases can be employed as intermediates and controls while developing inactive meganucleases.

As used herein, the term “LAGLIDADG meganuclease” refers either to meganucleases including a single LAGLIDADG motif, which are naturally dimeric, or to meganucleases including two LAGLIDADG motifs, which are naturally monomeric. The term “mono-LAGLIDADG meganuclease” is used herein to refer to meganucleases including a single LAGLIDADG motif, and the term “di-LAGLIDADG meganuclease” is used herein to refer to meganucleases including two LAGLIDADG motifs, when it is necessary to distinguish between the two. Each of the two structural domains of a di-LAGLIDADG meganuclease which includes a LAGLIDADG motif can be referred to as a LAGLIDADG subunit.

As used herein, the term “rationally-designed” means non-naturally occurring and/or genetically engineered. The rationally-designed meganucleases described herein differ from wild-type or naturally-occurring meganucleases in their amino acid sequence or primary structure, and may also differ in their secondary, tertiary or quaternary structure. In addition, the rationally-designed meganucleases described herein also differ from wild-type or naturally-occurring meganucleases in recognition sequence-specificity, affinity and/or activity.

As used herein, with respect to a protein, the term “recombinant” means having an altered amino acid sequence as a result of the application of genetic engineering techniques to nucleic acids which encode the protein, and cells or organisms which express the protein. With respect to a nucleic acid, the term “recombinant” means having an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant.

As used herein with respect to recombinant proteins, the term “modification” means any insertion, deletion or substitution of an amino acid residue in the recombinant sequence relative to a reference sequence (e.g., a wild-type).

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein, the term “wild-type” refers to any naturally-occurring form of a meganuclease. The term “wild-type” is not intended to mean the most common allelic variant of the enzyme in nature but, rather, any allelic variant found in nature. Wild-type meganucleases are distinguished from recombinant or non-naturally-occurring meganucleases.

As used herein, the term “recognition sequence half-site” or simply “half site” means a nucleic acid sequence in a double-stranded DNA molecule which is recognized by a monomer of a mono-LAGLIDADG meganuclease or by one LAGLIDADG subunit of a di-LAGLIDADG meganuclease.

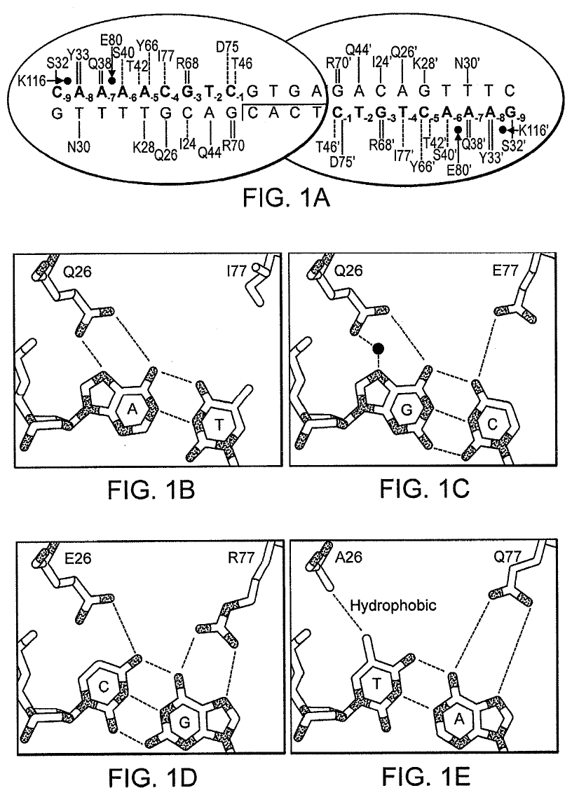

As used herein, the term “recognition sequence” refers to a pair of half-sites which is bound by either a mono-LAGLIDADG meganuclease dimer or a di-LAGLIDADG meganuclease monomer. The two half-sites may or may not be separated by base pairs that are not specifically recognized by the enzyme. In the cases of I-CreI, I-MsoI and I-CeuI, the recognition sequence half-site of each monomer spans 9 base pairs, and the two half-sites are separated by four base pairs which are not recognized specifically but which constitute the actual cleavage site (which has a 4 base pair overhang). Thus, the combined recognition sequences of the I-CreI, I-MsoI and I-CeuI meganuclease dimers normally span 22 base pairs, including two 9 base pair half-sites flanking a 4 base pair cleavage site. The base pairs of each half-site are designated −9 through −1, with the −9 position being most distal from the cleavage site and the −1 position being adjacent to the 4 central base pairs, which are designated N₁-N₄. The strand of each half-site which is oriented 5′ to 3′ in the direction from −9 to −1 (i.e., towards the cleavage site), is designated the “sense” strand and the opposite strand is designated the “antisense strand”, although neither strand may encode protein. Thus, the “sense” strand of one half-site is the antisense strand of the other half-site. See, for example, FIG. 1(A). In the case of the I-SceI meganuclease, which is a di-LAGLIDADG meganuclease monomer, the recognition sequence is an approximately 18 bp non-palindromic sequence, and there are no central base pairs which are not specifically recognized. By convention, one of the two strands is referred to as the “sense” strand and the other the “antisense” strand, although neither strand may encode protein. Even for meganucleases which have been inactivated and, therefore, do not cleave DNA, this numbering convention for the base pairs relative to the cleavage site will be retained herein.

As used herein, the term “specificity” means the ability of a meganuclease to recognize double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific meganuclease is capable of binding only one or a very few recognition sequences. For catalytically active meganucleases, specificity can be determined in a cleavage assay as described in Example 1. For inactive meganucleases, binding assays can be substituted. As used herein, a meganuclease has “altered” specificity if it binds to a recognition sequence which is not bound to by a reference meganuclease (e.g., a wild-type) or if the affinity of binding of a recognition sequence is increased or decreased by a significant (10-fold or more) amount relative to a reference meganuclease.

As used herein, the term “degeneracy” means the opposite of “specificity.” A highly-degenerate meganuclease is capable of binding a large number of divergent recognition sequences. A meganuclease can have sequence degeneracy at a single position within a half-site or at multiple, even all, positions within a half-site. Such sequence degeneracy can result from (i) the inability of any amino acid in the DNA-binding domain of a meganuclease to make a specific contact with any base at one or more positions in the recognition sequence, (ii) the ability of one or more amino acids in the DNA-binding domain of a meganuclease to make specific contacts with more than one base at one or more positions in the recognition sequence, and/or (iii) sufficient non-specific DNA binding affinity. A “completely” degenerate position can be occupied by any of the four bases and can be designated with an “N” in a half-site. A “partially” degenerate position can be occupied by two or three of the four bases (e.g., either purine (Pu), either pyrimidine (Py), or not G).

As used herein with respect to meganucleases, the term “DNA-binding affinity” or “binding affinity” means the tendency of a meganuclease to non-covalently associate with a reference DNA molecule (e.g., a recognition sequence or an arbitrary sequence). Binding affinity can be measured by a dissociation constant, K_{D} (e.g., the K_{D} of I-CreI for the WT recognition sequence is approximately 0.1 nM). As used herein, a meganuclease has “altered” binding affinity if the K_{D} of the recombinant meganuclease for a reference recognition sequence is increased or decreased by a significant (10-fold or more) amount relative to a reference meganuclease. For example, the DNA-binding affinity of a polypeptide can be determined, for example, by filter-binding, electrophoretic mobility-shift, or immunoprecipitation assays, as well as by any other methods known in the art.

As used herein with respect to meganuclease monomers, the term “affinity for dimer formation” means the tendency of a meganuclease monomer to non-covalently associate with a reference meganuclease monomer. The affinity for dimer formation can be measured with the same monomer (i.e., homodimer formation) or with a different monomer (i.e., heterodimer formation) such as a reference wild-type meganuclease. Binding affinity can be measured by a dissociation constant, K_{D}. As used herein, a meganuclease has “altered” affinity for dimer formation if the K_{D} of the recombinant meganuclease monomer for a reference meganuclease monomer is increased or decreased by a significant (10-fold or more) amount relative to a reference meganuclease monomer.

As used herein, the term “palindromic” refers to a recognition sequence consisting of inverted repeats of identical half-sites. In this case, however, the palindromic sequence need not be palindromic with respect to the four central base pairs, which are not contacted by the enzyme. In the case of dimeric meganucleases, palindromic DNA sequences are recognized by homodimers in which the two monomers make contacts with identical half-sites.

As used herein, the term “pseudo-palindromic” refers to a recognition sequence consisting of inverted repeats of non-identical or imperfectly palindromic half-sites. In this case, the pseudo-palindromic sequence not only need not be palindromic with respect to the four central base pairs, but also can deviate from a palindromic sequence between the two half-sites. Pseudo-palindromic DNA sequences are typical of the natural DNA sites recognized by wild-type homodimeric meganucleases in which two identical enzyme monomers make contacts with different half-sites.

As used herein, the term “non-palindromic” refers to a recognition sequence composed of two unrelated half-sites of a meganuclease. In this case, the non-palindromic sequence need not be palindromic with respect to either the four central base pairs or the two monomer half-sites. Non-palindromic DNA sequences are recognized by either di-LAGLIDADG meganucleases, highly degenerate mono-LAGLIDADG meganucleases (e.g., I-CeuI) or by heterodimers of mono-LAGLIDADG meganuclease monomers that recognize non-identical half-sites.

As used herein, the term “activity” refers to the rate at which a meganuclease of described herein cleaves a particular recognition sequence. Such activity is a measurable enzymatic reaction, involving the hydrolysis of phosphodiester bonds of double-stranded DNA. The activity of a meganuclease acting on a particular DNA substrate is affected by the affinity or avidity of the meganuclease for that particular DNA substrate which is, in turn, affected by both sequence-specific and non-sequence-specific interactions with the DNA. In inactive meganucleases, this activity is lacking.

As used herein, a meganuclease which is “inactive,” “inactivated” or “lacks catalytic activity” refers to a genetically-engineered meganuclease DNA-binding domain which cleaves the cleavage site of the wild-type enzyme at a rate that is reduced at least 10-fold, at least 100-fold, or at least 1,000-fold, when compared to the wild-type enzyme under the same cleavage conditions, or which does not cleave the cleavage site of the wild-type enzyme at all. If no cleavage of the cleavage site of the wild-type enzyme can be observed, it is said that such cleavage is “abolished.”

As used herein, the term “homologous recombination” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell. Thus, a catalytically active meganuclease can be used to cleave a recognition sequence within a target sequence and an exogenous nucleic acid with homology to or substantial sequence similarity with the target sequence can be delivered into the cell and used as a template for repair by homologous recombination. The DNA sequence of the exogenous nucleic acid, which may differ significantly from the target sequence, is thereby incorporated into the chromosomal sequence. The process of homologous recombination occurs primarily in eukaryotic organisms. The term “homology” is used herein as equivalent to “sequence similarity” and is not intended to require identity by descent or phylogenetic relatedness.

As used herein, the term “non-homologous end-joining” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. Thus, a catalytically active meganuclease can be used to produce a double-stranded break at a meganuclease recognition sequence within a target sequence to disrupt a gene (e.g., by introducing base insertions, base deletions, or frameshift mutations) by non-homologous end-joining. An exogenous nucleic acid lacking homology to or substantial sequence similarity with the target sequence may be captured at the site of a meganuclease-stimulated double-stranded DNA break by non-homologous end-joining (see, e.g. Salomon, et al. (1998), EMBO J. 17:6086-6095). The process of non-homologous end-joining occurs in both eukaryotes and prokaryotes such as bacteria.

As used herein, the term “sequence of interest” means any nucleic acid sequence, whether it codes for a protein, RNA, or regulatory element (e.g., an enhancer, silencer, or promoter sequence), that can be inserted into a genome or used to replace a genomic DNA sequence using a catalytically active meganuclease protein. Sequences of interest can have heterologous DNA sequences that allow for tagging a protein or RNA that is expressed from the sequence of interest. For instance, a protein can be tagged with tags including, but not limited to, an epitope (e.g., c-myc, FLAG) or other ligand (e.g., poly-His). Furthermore, a sequence of interest can encode a fusion protein, according to techniques known in the art (see, e.g., Ausubel et al., Current Protocols in Molecular Biology, Wiley 1999). In some cases, the sequence of interest is flanked by a DNA sequence that is recognized by a catalytically active meganuclease for cleavage. Thus, the flanking sequences are cleaved allowing for proper insertion of the sequence of interest into genomic recognition sequences cleaved by the active meganuclease. In some cases, the entire sequence of interest is homologous to or has substantial sequence similarity with the a target sequence in the genome such that homologous recombination effectively replaces the target sequence with the sequence of interest. In other embodiments, the sequence of interest is flanked by DNA sequences with homology to or substantial sequence similarity with the target sequence such that homologous recombination inserts the sequence of interest within the genome at the locus of the target sequence. In some embodiments, the sequence of interest is substantially identical to the target sequence except for mutations or other modifications in a meganuclease recognition sequence such that an active meganuclease can not cleave the target sequence after it has been modified by the sequence of interest.

As used herein, the term “targeted transcriptional effector” refers to a non-natural protein comprising a first domain comprising a non-naturally-occurring, rationally-designed meganuclease that has been modified relative to a wild-type meganuclease and a second domain comprising a natural or non-natural transcription effector domain. The first domain comprises a non-naturally-occurring, rationally-designed meganuclease that has been modified relative to a wild-type meganuclease with respect to DNA-binding specificity, DNA-binding affinity, and/or the ability to form heterodimers, and which has been inactivated with respect to its ability to cleave DNA. Such an inactive meganuclease is referred to as a “meganuclease DNA-binding domain.” The second domain comprises a natural or non-natural transcription effector domain. Such a transcription effector domain is able to interact directly or indirectly with the transcription machinery of a cell to either increase or decrease gene expression. The first and the second domains of a targeted transcriptional effectors can be fused together, or they can be connected through a flexible linker.

As used herein, the term “domain linker” means a chemical moiety which covalently joins a rationally-designed meganuclease DNA-binding domain and an effector domain (e.g., a transcription effector domain), having a backbone of chemical bonds forming a continuous connection between the peptides, and having a plurality of freely rotating bonds along that backbone. In certain embodiments, the domain linkers described herein have a backbone length (i.e., the sum of the bond lengths forming a continuous connection between the peptides) of at least about 13 Å. In some embodiments, a domain linker comprises a plurality of amino acid residues but this need not be the case. In specific embodiments, domain linkers are polypeptide linkers comprising 3-15 amino acid residues. Such domain linkers will have backbone lengths of approximately 13-65 Å.

The domain linkers can be substantially linear, biochemically inert, hydrophilic and/or non-cleavable by proteases, but branched domain linkers, or linkers with reactive moieties, hydrophobic residues and protease cleavage sites may be suitable for certain embodiments. The domain linkers can also be designed to lack secondary structure under physiological conditions. Thus, for example, the domain linker sequences can be composed of a plurality of residues selected from the group consisting of glycine, serine, threonine, cysteine, asparagine, glutamine, and proline.

In some embodiments, domain linkers consist essentially of glycine and serine residues. Domain linkers including the larger, aromatic residues may also be included, although they may cause steric hindrance. Similarly, the charged amino acids may be included, but they may interact to form secondary structures, and the nonpolar amino acids may be included, but they may decrease solubility. Domain linkers which do not satisfy one or more of these criteria may prove to be at least as effective in some embodiments.

For chemical synthesis of domain linkers, one of skill in the art of organic synthesis may design a wide variety of linkers which satisfy the requirements discussed above. Thus, depending upon the nature of the termini to be joined (i.e., N- and/or C-termini), appropriate end groups are chosen for the linker such that the linker may be joined to the chosen termini of the two proteins to be fused (e.g., using a naturally occurring amino acid, D-isomer amino acid, or modified amino acid, such as sarcosine or D-alanine, at one or both ends).

In some embodiments, domain linkers include polymers or copolymers of organic acids, aldehydes, alcohols, thiols, amines and the like. For example, polymers or copolymers of hydroxy-, amino-, or di-carboxylic acids, such as glycolic acid, lactic acid, sebacic acid, or sarcosine may be employed. Alternatively, polymers or copolymers of saturated or unsaturated hydrocarbons such as ethylene glycol, propylene glycol, saccharides, and the like may be employed. One example of such a domain linker is polyethylene glycol (with or without, e.g., D-alanine at the ends), available from Shearwater Polymers, Inc. (Huntsville, AL). These linkers can optionally have amide linkages, sulfhydryl linkages, or heterofunctional linkages. Other examples include polymers or copolymers of non-naturally occurring amino acids (including, for example, D-isomers). Certain non-naturally occurring amino acids have characteristics which may be advantageous in connection with the present invention. For example, N-methyl glycine (sarcosine) would be predicted to minimize hydrogen bonding and secondary structure formation while exhibiting favorable solubility characteristics and, therefore, a polysarcosine linker (with or without, e.g., lysine at the ends) may be employed. These and many other domain linkers may be readily employed by one of ordinary skill in the art using traditional techniques of chemical synthesis.

Alternatively, domain linkers can be rationally designed using computer program capable of modeling both DNA-binding sites and the peptides themselves (Desjarlais & Berg (1993), Proc. Natl. Acad. Sci. USA 90:2256-2260 (1993), Desjarlais & Berg (1994), Proc. Natl. Acad. Sci. USA 91:11099-11103), or by phage display methods.

In other embodiments, non-covalent methods can be used to produce molecules with meganuclease DNA-binding domains associated with effector domains.

In addition to regulatory domains, a meganuclease DNA-binding domain can be expressed as a fusion protein such as maltose binding protein (“MBP”), glutathione S transferase (GST), hexahistidine, c-myc, and the FLAG epitope, for ease of purification, monitoring expression, or monitoring cellular and subcellular localization.

As used herein, the term “single-chain meganuclease” refers to a non-naturally-occurring meganuclease comprising a pair of mono-LAGLIDADG meganucleases that are covalently joined into a single polypeptide using an amino acid linker. For example, a pair of rationally-designed meganucleases derived from I-CreI may be joined using an amino acid linker to join a first rationally-designed meganuclease monomer with a second rationally designed meganuclease monomer to produce a single-chain heterodimer (see, e.g., Example 5). Single-chain meganucleases typically comprise a pair of rationally-designed meganuclease subunits that recognize different half-sites such that the recognition sequence for a single-chain meganuclease is non-palindromic.

As used herein with respect to both amino acid sequences and nucleic acid sequences, the terms “percentage similarity” and “sequence similarity” refer to a measure of the degree of similarity of two sequences based upon an alignment of the sequences which maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7 (1-2): 203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

As used herein with respect to modifications of two proteins or amino acid sequences, the term “corresponding to” is used to indicate that a specified modification in the first protein is a substitution of the same amino acid residue as in the modification in the second protein, and that the amino acid position of the modification in the first proteins corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment, and despite the fact that X and Y may be different numbers.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

As used herein, unless specifically indicated otherwise, the word “or” is used in the inclusive sense of “and/or” and not the exclusive sense of “either/or.”

### 2.1 Rationally-Designed Meganucleases with Altered Sequence-Specificity

In one aspect of the invention, methods for rationally designing recombinant LAGLIDADG family meganucleases are provided. In this aspect, recombinant meganucleases are rationally-designed by first predicting amino acid substitutions that can alter base preference at each position in the half-site. These substitutions can be experimentally validated individually or in combinations to produce meganucleases with the desired cleavage specificity.

In accordance with the invention, amino acid substitutions that can cause a desired change in base preference are predicted by determining the amino acid side chains of a reference meganuclease (e.g., a wild-type meganuclease, or a non-naturally-occurring reference meganuclease) that are able to participate in making contacts with the nucleic acid bases of the meganuclease's DNA recognition sequence and the DNA phosphodiester backbone, and the spatial and chemical nature of those contacts. These amino acids include but are not limited to side chains involved in contacting the reference DNA half-site. Generally, this determination requires having knowledge of the structure of the complex between the meganuclease and its double-stranded DNA recognition sequence, or knowledge of the structure of a highly similar complex (e.g., between the same meganuclease and an alternative DNA recognition sequence, or between an allelic or phylogenetic variant of the meganuclease and its DNA recognition sequence).

Three-dimensional structures, as described by atomic coordinates data, of a polypeptide or complex of two or more polypeptides can be obtained in several ways. For example, protein structure determinations can be made using techniques including, but not limited to, X-ray crystallography, NMR, and computer simulations. Another approach is to analyze databases of existing structural co-ordinates for the meganuclease of interest or a related meganuclease. Such structural data is often available from databases in the form of three-dimensional coordinates. Often this data is accessible through online databases (e.g., the RCSB Protein Data Bank at www.rcsb.org/pdb).

Structural information can be obtained experimentally by analyzing the diffraction patterns of, for example, X-rays or electrons, created by regular two- or three-dimensional arrays (e.g., crystals) of proteins or protein complexes. Computational methods are used to transform the diffraction data into three-dimensional atomic co-ordinates in space. For example, the field of X-ray crystallography has been used to generate three-dimensional structural information on many protein-DNA complexes, including meganucleases (see, e.g., Chevalier et al. (2001), Nucleic Acids Res. 29 (18): 3757-3774).

Nuclear Magnetic Resonance (NMR) also has been used to determine inter-atomic distances of molecules in solution. Multi-dimensional NMR methods combined with computational methods have succeeded in determining the atomic co-ordinates of polypeptides of increasing size (see, e.g., Tzakos et al. (2006), Annu. Rev. Biophys. Biomol. Struct. 35:19-42.).

Alternatively, computational modeling can be used by applying algorithms based on the known primary structures and, when available, secondary, tertiary and/or quaternary structures of the protein/DNA, as well as the known physiochemical nature of the amino acid side chains, nucleic acid bases, and bond interactions. Such methods can optionally include iterative approaches, or experimentally-derived constraints. An example of such computational software is the CNS program described in Adams et al. (1999), Acta Crystallogr. D. Biol. Crystallogr. 55 (Pt 1): 181-90. A variety of other computational programs have been developed that predict the spatial arrangement of amino acids in a protein structure and predict the interaction of the amino acid side chains of the protein with various target molecules (see, e.g., U.S. Pat. No. 6,988,041).

Thus, in some embodiments of the invention, computational models are used to identify specific amino acid residues that specifically interact with DNA nucleic acid bases and/or facilitate non-specific phosphodiester backbone interactions. For instance, computer models of the totality of the potential meganuclease-DNA interaction can be produced using a suitable software program, including, but not limited to, MOLSCRIPT™ 2.0 (Avatar Software AB, Stockholm, Sweden), the graphical display program O (Jones et. al. (1991), Acta Crystallography, A47: 110), the graphical display program GRASP™ (Nicholls et al. (1991), PROTEINS, Structure, Function and Genetics 11 (4): 281ff), or the graphical display program INSIGHT™ (TSI, Inc., Shoreview, MN). Computer hardware suitable for producing, viewing and manipulating three-dimensional structural representations of protein-DNA complexes are commercially available and well known in the art (e.g., Silicon Graphics Workstation, Silicon Graphics, Inc., Mountainview, CA).

Specifically, interactions between a meganuclease and its double-stranded DNA recognition sequences can be resolved using methods known in the art. For example, a representation, or model, of the three dimensional structure of a multi-component complex structure, for which a crystal has been produced, can be determined using techniques which include molecular replacement or SIR/MIR (single/multiple isomorphous replacement) (see, e.g., Brunger (1997), Meth. Enzym. 276:558-580; Navaza and Saludjian (1997), Meth. Enzym. 276:581-594; Tong and Rossmann (1997), Meth. Enzym. 276:594-611; and Bentley (1997), Meth. Enzym. 276:611-619) and can be performed using a software program, such as AMoRe/Mosflm (Navaza (1994), Acta Cryst. A 50: 157-163; CCP4 (1994), Acta Cryst. D 50: 760-763) or XPLOR (see, Brünger et al. (1992), X-PLOR Version 3.1. A System for X-ray Crystallography and NMR, Yale University Press, New Haven, CT).

The determination of protein structure and potential meganuclease-DNA interaction allows for rational choices concerning the amino acids that can be changed to affect enzyme activity and specificity. Decisions are based on several factors regarding amino acid side chain interactions with a particular base or DNA phosphodiester backbone. Chemical interactions used to determine appropriate amino acid substitutions include, but are not limited to, van der Waals forces, steric hindrance, ionic bonding, hydrogen bonding, and hydrophobic interactions. Amino acid substitutions can be selected which either favor or disfavor specific interactions of the meganuclease with a particular base in a potential recognition sequence half-site in order to increase or decrease specificity for that sequence and, to some degree, overall binding affinity and activity. In addition, amino acid substitutions can be selected which either increase or decrease binding affinity for the phosphodiester backbone of double-stranded DNA in order to increase or decrease overall activity and, to some degree, to decrease or increase specificity.

Thus, in specific embodiments, a three-dimensional structure of a meganuclease-DNA complex is determined and a “contact surface” is defined for each base-pair in a DNA recognition sequence half-site. In some embodiments, the contact surface comprises those amino acids in the enzyme with β-carbons less than 9.0 Å from a major groove hydrogen-bond donor or acceptor on either base in the pair, and with side chains oriented toward the DNA, irrespective of whether the residues make base contacts in the wild-type meganuclease-DNA complex. In other embodiments, residues can be excluded if the residues do not make contact in the wild-type meganuclease-DNA complex, or residues can be included or excluded at the discretion of the designer to alter the number or identity of the residues considered. In one example, as described below, for base positions −2, −7, −8, and −9 of the wild-type I-CreI half-site, the contact surfaces were limited to the amino acid positions that actually interact in the wild-type enzyme-DNA complex. For positions −1, −3, −4, −5, and −6, however, the contact surfaces were defined to contain additional amino acid positions that are not involved in wild-type contacts but which could potentially contact a base if substituted with a different amino acid.

It should be noted that, although a recognition sequence half-site is typically represented with respect to only one strand of DNA, meganucleases bind in the major groove of double-stranded DNA, and make contact with nucleic acid bases on both strands. In addition, the designations of “sense” and “antisense” strands are completely arbitrary with respect to meganuclease binding and recognition. Sequence specificity at a position can be achieved either through interactions with one member of a base pair, or by a combination of interactions with both members of a base pair. Thus, for example, in order to favor the presence of an A/T base pair at position X, where the A base is on the “sense” strand and the T base is on the “antisense” strand, residues are selected which are sufficiently close to contact the sense strand at position X and which favor the presence of an A, and/or residues are selected which are sufficiently close to contact the antisense strand at position X and which favor the presence of a T. In accordance with the invention, a residue is considered sufficiently close if the β-carbon of the residue is within 9 Å of the closest atom of the relevant base.

Thus, for example, an amino acid with a β-carbon within 9 Å of the DNA sense strand but greater than 9 Å from the antisense strand is considered for potential interactions with only the sense strand. Similarly, an amino acid with a β-carbon within 9 Å of the DNA antisense strand but greater than 9 Å from the sense strand is considered for potential interactions with only the antisense strand. Amino acids with β-carbons that are within 9 Å of both DNA strands are considered for potential interactions with either strand.

For each contact surface, potential amino acid substitutions are selected based on their predicted ability to interact favorably with one or more of the four DNA bases. The selection process is based upon two primary criteria: (i) the size of the amino acid side chains, which will affect their steric interactions with different nucleic acid bases, and (ii) the chemical nature of the amino acid side chains, which will affect their electrostatic and bonding interactions with the different nucleic acid bases.

With respect to the size of side chains, amino acids with shorter and/or smaller side chains can be selected if an amino acid β-carbon in a contact surface is <6 Å from a base, and amino acids with longer and/or larger side chains can be selected if an amino acid β-carbon in a contact surface is >6 Å from a base. Amino acids with side chains that are intermediate in size can be selected if an amino acid β-carbon in a contact surface is 5-8 Å from a base.

The amino acids with relatively shorter and smaller side chains can be assigned to Group 1, including glycine (G), alanine (A), serine(S), threonine (T), cysteine (C), valine (V), leucine (L), isoleucine (I), aspartate (D), asparagine (N) and proline (P). Proline, however, is expected to be used less frequently because of its relative inflexibility. In addition, glycine is expected to be used less frequently because it introduces unwanted flexibility in the peptide backbone and its very small size reduces the likelihood of effective contacts when it replaces a larger residue. On the other hand, glycine can be used in some instances for promoting a degenerate position. The amino acids with side chains of relatively intermediate length and size can be assigned to Group 2, including lysine (K), methionine (M), arginine (R), glutamate (E) and glutamine (Q). The amino acids with relatively longer and/or larger side chains can be assigned to Group 3, including lysine (K), methionine (M), arginine (R), histidine (H), phenylalanine (F), tyrosine (Y), and tryptophan (W). Tryptophan, however, is expected to be used less frequently because of its relative inflexibility. In addition, the side chain flexibility of lysine, arginine, and methionine allow these amino acids to make base contacts from long or intermediate distances, warranting their inclusion in both Groups 2 and 3. These groups are also shown in tabular form below:

| Group 1 | Group 2 | Group 3 |
|---|---|---|
| glycine (G) | glutamine (Q) | arginine (R) |
| alanine (A) | glutamate (E) | histidine (H) |
| serine (S) | lysine (K) | phenylalanine (F) |
| threonine (T) | methionine (M) | tyrosine (Y) |
| cysteine (C) | arginine (R) | tryptophan (W) |
| valine (V) | | lysine (K) |
| leucine (L) | | methionine (M) |
| isoleucine (I) | | |
| aspartate (D) | | |
| asparagine (N) | | |
| proline (P) | | |

With respect to the chemical nature of the side chains, the different amino acids are evaluated for their potential interactions with the different nucleic acid bases (e.g., van der Waals forces, ionic bonding, hydrogen bonding, and hydrophobic interactions) and residues are selected which either favor or disfavor specific interactions of the meganuclease with a particular base at a particular position in the double-stranded DNA recognition sequence half-site. In some instances, it may be desired to create a half-site with one or more complete or partial degenerate positions. In such cases, one may choose residues which favor the presence of two or more bases, or residues which disfavor one or more bases. For example, partial degenerate base recognition can be achieved by sterically hindering a pyrimidine at a sense or antisense position.

Recognition of guanine (G) bases is achieved using amino acids with basic side chains that form hydrogen bonds to N7 and 06 of the base. Cytosine (C) specificity is conferred by negatively-charged side chains which interact unfavorably with the major groove electronegative groups present on all bases except C. Thymine (T) recognition is rationally-designed using hydrophobic and van der Waals interactions between hydrophobic side chains and the major groove methyl group on the base. Finally, adenine (A) bases are recognized using the carboxamide side chains Asn and Gln or the hydroxyl side chain of Tyr through a pair of hydrogen bonds to N7 and N6 of the base. Lastly, His can be used to confer specificity for a purine base (A or G) by donating a hydrogen bond to N7. These straightforward rules for DNA recognition can be applied to predict contact surfaces in which one or both of the bases at a particular base-pair position are recognized through a rationally-designed contact.

Thus, based on their binding interactions with the different nucleic acid bases, and the bases which they favor at a position with which they make contact, each amino acid residue can be assigned to one or more different groups corresponding to the different bases they favor (i.e., G, C, T or A). Thus, Group G includes arginine (R), lysine (K) and histidine (H); Group C includes aspartate (D) and glutamate (E); Group T includes alanine (A), valine (V), leucine (L), isoleucine (I), cysteine (C), threonine (T), methionine (M) and phenylalanine (F); and Group A includes asparagine (N), glutamine (N), tyrosine (Y) and histidine (H). Note that histidine appears in both Group G and Group A; that serine(S) is not included in any group but may be used to favor a degenerate position; and that proline, glycine, and tryptophan are not included in any particular group because of predominant steric considerations. These groups are also shown in tabular form below:

| Group G | Group C | Group T | Group A |
|---|---|---|---|
| arginine (R) | aspartate (D) | alanine (A) | asparagine (N) |
| lysine (K) | glutamate (E) | valine (V) | glutamine (Q) |
| histidine (H) | | leucine (L) | tyrosine (Y) |
| | | isoleucine (I) | histidine (H) |
| | | cysteine (C) | |
| | | threonine (T) | |
| | | methionine (M) | |
| | | phenylalanine (F) | |

Thus, in accordance with the invention, in order to effect a desired change in the recognition sequence half-site of a meganuclease at a given position X, (1) determine at least the relevant portion of the three-dimensional structure of the wild-type or reference meganuclease-DNA complex and the amino acid residue side chains which define the contact surface at position X; (2) determine the distance between the β-carbon of at least one residue comprising the contact surface and at least one base of the base pair at position X; and (3) (a) for a residue which is <6 Å from the base, select a residue from Group 1 and/or Group 2 which is a member of the appropriate one of Group G, Group C, Group T or Group A to promote the desired change, and/or (b) for a residue which is >6 Å from the base, select a residue from Group 2 and/or Group 3 which is a member of the appropriate one of Group G, Group C, Group T or Group A to promote the desired change. More than one such residue comprising the contact surface can be selected for analysis and modification and, in some embodiments, each such residue is analyzed and multiple residues are modified. Similarly, the distance between the β-carbon of a residue included in the contact surface and each of the two bases of the base pair at position X can be determined and, if the residue is within 9 Å of both bases, then different substitutions can be made to affect the two bases of the pair (e.g., a residue from Group 1 to affect a proximal base on one strand, or a residue from Group 3 to affect a distal base on the other strand). Alternatively, a combination of residue substitutions capable of interacting with both bases in a pair can affect the specificity (e.g., a residue from the T Group contacting the sense strand combined with a residue from the A Group contacting the antisense strand to select for T/A). Finally, multiple alternative modifications of the residues can be validated either empirically (e.g., by producing the recombinant meganuclease and testing its sequence recognition) or computationally (e.g., by computer modeling of the meganuclease-DNA complex of the modified enzyme) to choose amongst alternatives.

Once one or more desired amino acid modifications of the wild-type or reference meganuclease are selected, the rationally-designed meganuclease can be produced by recombinant methods and techniques well known in the art. In some embodiments, non-random or site-directed mutagenesis techniques are used to create specific sequence modifications. Non-limiting examples of non-random mutagenesis techniques include overlapping primer PCR (see, e.g., Wang et al. (2006), Nucleic Acids Res. 34 (2): 517-527), site-directed mutagenesis (see, e.g., U.S. Pat. No. 7,041,814), cassette mutagenesis (see, e.g., U.S. Pat. No. 7,041,814), and the manufacturer's protocol for the Altered Sites® II Mutagenesis Systems kit commercially available from Promega Biosciences, Inc. (San Luis Obispo, CA).

The recognition and cleavage of a specific DNA sequence by a rationally-designed meganuclease can be assayed by any method known by one skilled in the art (see, e.g., U.S. Pat. Pub. No. 2006/0078552). In certain embodiments, the determination of meganuclease cleavage is determined by in vitro cleavage assays. Such assays use in vitro cleavage of a polynucleotide substrate comprising the intended recognition sequence of the assayed meganuclease and, in certain embodiments, variations of the intended recognition sequence in which one or more bases in one or both half-sites have been changed to a different base. Typically, the polynucleotide substrate is a double-stranded DNA molecule comprising a target site which has been synthesized and cloned into a vector. The polynucleotide substrate can be linear or circular, and typically comprises only one recognition sequence. The meganuclease is incubated with the polynucleotide substrate under appropriate conditions, and the resulting polynucleotides are analyzed by known methods for identifying cleavage products (e.g., electrophoresis or chromatography). If there is a single recognition sequence in a linear, double-strand DNA substrate, the meganuclease activity is detected by the appearance of two bands (products) and the disappearance of the initial full-length substrate band. In one embodiment, meganuclease activity can be assayed as described in, for example, Wang et al. (1997), Nucleic Acid Res., 25:3767-3776.

In other embodiments, the cleavage pattern of the meganuclease is determined using in vivo cleavage assays (see, e.g., U.S. Pat. Pub. No. 2006/0078552). In particular embodiments, the in vivo test is a single-strand annealing recombination test (SSA). This kind of test is known to those of skill in the art (Rudin et al. (1989), Genetics 122:519-534; Fishman-Lobell et al. (1992), Science 258:480-4).

As will be apparent to one of skill in the art, additional amino acid substitutions, insertions or deletions can be made to domains of the meganuclease enzymes other than those involved in DNA recognition and binding without complete loss of activity. Substitutions can be conservative substitutions of similar amino acid residues at structurally or functionally constrained positions, or can be non-conservative substitutions at positions which are less structurally or functionally constrained. Such substitutions, insertions and deletions can be identified by one of ordinary skill in the art by routine experimentation without undue effort. Thus, in some embodiments, the recombinant meganucleases described herein include proteins having anywhere from 85% to 99% sequence similarity (e.g., 85%, 87.5%, 90%, 92.5%, 95%, 97.5%, 99%) to a reference meganuclease sequence. With respect to each of the wild-type I-CreI, I-MsoI, I-SceI and I-CeuI proteins, the most N-terminal and C-terminal sequences are not clearly visible in X-ray crystallography studies, suggesting that these positions are not structurally or functionally constrained. Therefore, these residues can be excluded from calculation of sequence similarity, and the following reference meganuclease sequences can be used: residues 2-153 of SEQ ID NO: 1 for I-CreI, residues 6-160 of SEQ ID NO: 6 for I-MsoI, residues 3-186 of SEQ ID NO: 9 for I-SceI, and residues 5-211 of SEQ ID NO: 12 for I-CeuI.

### 2.2 LAGLIDADG Family Meganucleases

The LAGLIDADG meganuclease family is composed of more than 200 members from a diverse phylogenetic group of host organisms. All members of this family have one or two copies of a highly conserved LAGLIDADG motif along with other structural motifs involved in cleavage of specific DNA sequences. Enzymes that have a single copy of the LAGLIDADG motif (i.e., mono-LAGLIDADG meganucleases) function as dimers, whereas the enzymes that have two copies of this motif (i.e., di-LAGLIDADG meganucleases) function as monomers.

All LAGLIDADG family members recognize and cleave relatively long sequences (>12 bp), leaving four nucleotide 3′ overhangs. These enzymes also share a number of structural motifs in addition to the LAGLIDADG motif, including a similar arrangement of anti-parallel β-strands at the protein-DNA interface. Amino acids within these conserved structural motifs are responsible for interacting with the DNA bases to confer recognition sequence specificity. The overall structural similarity between some members of the family (e.g., I-CreI, I-MsoI, I-SceI and I-CeuI) has been elucidated by X-ray crystallography. Accordingly, the members of this family can be modified at particular amino acids within such structural motifs to change the overall activity or sequence-specificity of the enzymes, and corresponding modifications can reasonable be expected to have similar results in other family members. See, generally, Chevalier et al. (2001), Nucleic Acid Res. 29 (18): 3757-3774).

2.2.1 Rationally-Designed Meganucleases Derived from I-CreI

In one aspect, the present invention relates to non-naturally-occurring, rationally-designed meganucleases which are based upon or derived from the I-CreI meganuclease of Chlamydomonas reinhardtii. The wild-type amino acid sequence of the I-CreI meganuclease is shown in SEQ ID NO: 1, which corresponds to Genbank Accession #PO5725. Two recognition sequence half sites of the wild-type I-CreI meganuclease from crystal structure having PDB identifier (PDB ID) 1BP7 are shown below:

| | |
|---|---|
| Position | -9-8-7-6-5-4-3-2-1 |
| | 5′-G A A A C T G T C T C A C G A C G T T T T G-3′ SEQ ID NO: 2 |
| | 3′-C T T T G A C A G A G T G C T G C A A A A C-5′ SEQ ID NO: 3 |
| Position | -1-2-3-4-5-6-7-8-9 |

Note that this natural recognition sequence is not perfectly palindromic, even outside the central four base pairs. The two recognition sequence half-sites are shown in bold on their respective sense strands.

Wild-type I-CreI also recognizes and cuts the following perfectly palindromic (except for the central N1-N4 bases) sequence:

| | |
|---|---|
| Position | -9-8-7-6-5-4-3-2-1 |
| | 5′-C A A A C T G T C G T G A G A C A G T T T G-3′ SEQ ID NO: 4 |
| | 3′-G T T T G A C A G C A C T C T G T C A A A C-5′ SEQ ID NO: 5 |
| Position | -1-2-3-4-5-6-7-8-9 |

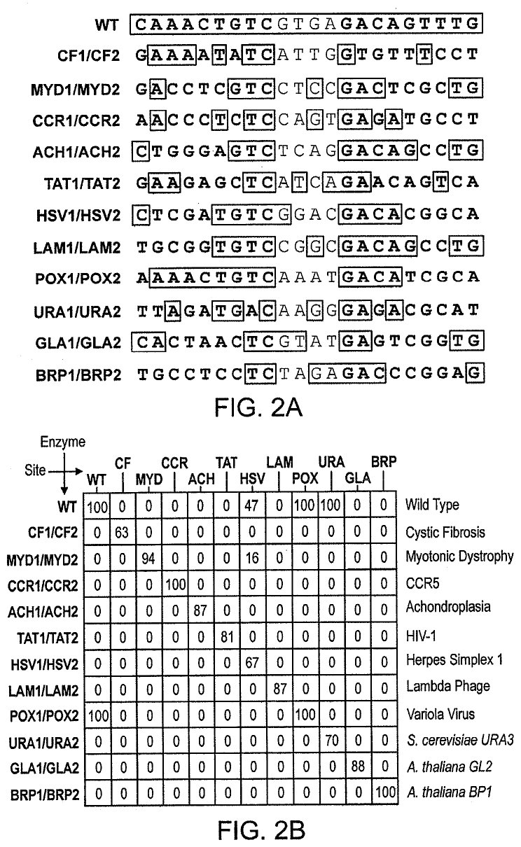

The palindromic sequence of SEQ ID NO: 4 and SEQ ID NO: 5 is considered to be a better substrate for the wild-type I-CreI because the enzyme binds this site with higher affinity and cleaves it more efficiently than the natural DNA sequence. For the purposes of the following disclosure, and with particular regard to the experimental results presented herein, this palindromic sequence cleaved by wild-type I-CreI is referred to as “WT” (see, e.g., FIG. 2(A)). The two recognition sequence half-sites are shown in bold on their respective sense strands.

FIG. 1(A) depicts the interactions of a wild-type I-CreI meganuclease homodimer with a double-stranded DNA recognition sequence, FIG. 1(B) shows the specific interactions between amino acid residues of the enzyme and bases at the −4 position of one half-site for a wild-type enzyme and one wild-type recognition sequence, and FIGS. 1(C)-(E) show the specific interactions between amino acid residues of the enzyme and bases at the −4 position of one half-site for three rationally-designed meganucleases described herein with altered specificity at position −4 of the half-site.

Thus, the base preference at any specified base position of the half-site can be rationally altered to each of the other three base pairs using the methods disclosed herein. First, the wild-type recognition surface at the specified base position is determined (e.g., by analyzing meganuclease-DNA complex co-crystal structures; or by computer modeling of the meganuclease-DNA complexes). Second, existing and potential contact residues are determined based on the distances between the β-carbons of the surrounding amino acid positions and the nucleic acid bases on each DNA strand at the specified base position. For example, and without limitation, as shown in FIG. 1(A), the I-CreI wild type meganuclease-DNA contact residues at position −4 involve a glutamine at position 26 which hydrogen bonds to an A base on the antisense DNA strand. Residue 77 was also identified as potentially being able to contact the −4 base on the DNA sense strand. The β-carbon of residue 26 is 5.9 Å away from N7 of the A base on the antisense DNA strand, and the β-carbon of residue 77 is 7.15 Å away from the C5-methyl of the T on the sense strand. According to the distance and base chemistry rules described herein, a C on the sense strand could hydrogen bond with a glutamic acid at position 77 and a G on the antisense strand could bond with glutamine at position 26 (mediated by a water molecule, as observed in the wild-type I-CreI crystal structure) (see FIG. 1(C)); a G on the sense strand could hydrogen bond with an arginine at position 77 and a C on the antisense strand could hydrogen bond with a glutamic acid at position 26 (see FIG. 1(D)); an A on the sense strand could hydrogen bond with a glutamine at position 77 and a T on the antisense strand could form hydrophobic contacts with an alanine at position 26 (see FIG. 1(E)). If the base specific contact is provided by position 77, then the wild-type contact, Q26, can be substituted (e.g., with a serine residue) to reduce or remove its influence on specificity. Alternatively, complementary mutations at positions 26 and 77 can be combined to specify a particular base pair (e.g., A26 specifies a T on the antisense strand and Q77 specifies an A on the sense strand (FIG. 1(E)). These predicted residue substitutions have all been validated experimentally.

Thus, in accordance with the invention, a substantial number of amino acid modifications to the DNA recognition domain of the I-CreI meganuclease have been identified which, singly or in combination, result in recombinant meganucleases with specificities altered at individual bases within the DNA recognition sequence half-site, such that these non-naturally-occurring, rationally-designed meganucleases have half-sites different from the wild-type enzyme. The amino acid modifications of I-CreI and the resulting change in recognition sequence half-site specificity are shown in Table 1:

TABLE 1

| | Favored Sense-Strand Base | | | | | | | | | | |
|---|---|---|---|---|---|---|---|---|---|---|---|
| Posn. | A | C | G | T | A/T | A/C | A/G | C/T | G/T | A/G/T | A/C/G/T |

| | | | | | | | | | | |
|---|---|---|---|---|---|---|---|---|---|---|
| −1 | Y75 | R70* | K70 | Q70* | | | | T46* | | G70 |
| | L75* | H75* | E70* | C70 | | | | | | A70 |
| | C75* | R75* | E75* | L70 | | | | | | S70 |
| | Y139* | H46* | E46* | Y75* | | | | | | G46* |
| | C46* | K46* | D46* | Q75* | | | | | | |
| | A46* | R46* | | H75* | | | | | | |
| | | | | H139 | | | | | | |
| | | | | Q46* | | | | | | |
| | | | | H46* | | | | | | |
| −2 | Q70 | E70 | H70 | Q44* | C44* | | | | | |
| | T44* | D70 | D44* | | | | | | | |
| | A44* | K44* | E44* | | | | | | | |
| | V44* | R44* | | | | | | | | |
| | I44* | | | | | | | | | |
| | L44* | | | | | | | | | |
| | N44* | | | | | | | | | |
| −3 | Q68 | E68 | R68 | M68 | | H68 | | Y68 | K68 | |
| | C24* | F68 | | C68 | | | | | | |
| | I24* | K24* | | L68 | | | | | | |
| | | R24* | | F68 | | | | | | |
| −4 | A26* | E77 | R77 | | | | | S77 | | S26* |
| | Q77 | K26* | E26* | | | | | Q26* | | |
| −5 | | E42 | R42 | | | K28* | C28* | | | M66 |
| | | | | | | | Q42 | | | K66 |
| −6 | Q40 | E40 | R40 | C40 | A40 | | | | | S40 |
| | C28* | R28* | | I40 | A79 | | | | | S28* |
| | | | | V40 | A28* | | | | | |
| | | | | C79 | H28* | | | | | |
| | | | | I79 | | | | | | |
| | | | | V79 | | | | | | |
| | | | | Q28* | | | | | | |
| −7 | N30* | E38 | K38 | I38 | | | C38 | | | H38 |
| | Q38 | K30* | R38 | L38 | | | | | | N38 |
| | | R30* | E30* | | | | | | | Q30* |
| −8 | F33 | E33 | F33 | L33 | | R32* | R33 | | | |
| | Y33 | D33 | H33 | V33 | | | | | | |
| | | | | I33 | | | | | | |
| | | | | F33 | | | | | | |
| | | | | C33 | | | | | | |
| −9 | | E32 | R32 | L32 | | | | D32 | | S32 |
| | | | K32 | V32 | | | | I32 | | N32 |
| | | | | A32 | | | | | | H32 |
| | | | | C32 | | | | | | Q32 |
| | | | | | | | | | | T32 |

Bold entries are wild-type contact residues and do not constitute “modifications” as used herein. An asterisk indicates that the residue contacts the base on the antisense strand.

2.2.2 Rationally-Designed Meganucleases Derived from I-MsoI

In another aspect, the present invention relates to non-naturally-occurring, rationally-designed meganucleases which are based upon or derived from the I-MsoI meganuclease of Monomastix sp. The wild-type amino acid sequence of the I-MsoI meganuclease is shown in SEQ ID NO: 6, which corresponds to Genbank Accession #AAL34387. Two recognition sequence half-sites of the wild-type I-MsoI meganuclease from crystal structure having PDB identifier (PDB ID) 1M5X are shown below:

| | |
|---|---|
| Position | -9-8-7-6-5-4-3-2-1 |
| | 5′-C A G A A C G T C G T G A G A C A G T T C C-3′ SEQ ID NO: 7 |
| | 3′-G T C T T G C A G C A C T C T G T C A A G G-5′ SEQ ID NO: 8 |
| Position | -1-2-3-4-5-6-7-8-9 |

Note that the recognition sequence is not perfectly palindromic, even outside the central four base pairs. The two recognition sequence half-sites are shown in bold on their respective sense strands.

In accordance with the invention, a substantial number of amino acid modifications to the DNA recognition domain of the I-MsoI meganuclease have been identified which, singly or in combination, can result in recombinant meganucleases with specificities altered at individual bases within the DNA recognition sequence half-sites, such that these non-naturally-occurring, rationally-designed meganucleases have recognition sequences different from the wild-type enzyme. Amino acid modifications of I-MsoI and the predicted change in recognition sequence half-site specificity are shown in Table 2:

TABLE 2

Favored Sense-Strand Base

| Position | A | C | G | T |
|---|---|---|---|---|
| −1 | K75* | D77 | K77 | C77 |
| | Q77 | E77 | R77 | L77 |
| | A49* | K49* | E49* | Q79* |
| | C49* | R75* | E79* | |
| | K79* | K75* | | |
| | | R79* | | |
| | | K79* | | |
| −2 | Q75 | E75 | K75 | A75 |
| | K81 | D75 | E47* | C75 |
| | C47* | R47* | E81* | V75 |
| | I47* | K47* | | I75 |
| | L47* | K81* | | T75 |
| | | R81* | | Q47* |
| | | | | Q81* |
| −3 | Q72 | E72 | R72 | K72 |
| | C26* | Y72 | K72 | Y72 |
| | L26* | H26* | Y26* | H26* |
| | V26* | K26* | F26* | |
| | A26* | R26* | | |
| | I26* | | | |
| −4 | K28 | K28* | R83 | K28 |
| | Q83 | R28* | K83 | K83 |
| | | E83 | | Q28* |
| −5 | K28 | K28* | R45 | Q28* |
| | C28* | R28* | E28* | |
| | L28* | | | |
| | 128* | | | |
| −6 | I30* | E43 | R43 | K43 |
| | V30* | E85 | K43 | I85 |
| | S30* | K30* | K85 | V85 |
| | L30* | R30* | R85 | L85 |
| | Q43 | | E30* | Q30* |
| | | | D30* | |
| −7 | Q41 | E32 | R32 | K32 |
| | | E41 | R41 | M41 |
| | | | K41 | L41 |
| | | | | I41 |
| −8 | Y35 | E32 | R32 | K32 |
| | K35 | | K32 | K35 |
| | | | K35 | |
| | | | R35 | |
| −9 | N34 | D34 | K34 | S34 |
| | H34 | E34 | R34 | C34 |
| | | S34 | H34 | V34 |
| | | | | T34 |
| | | | | A34 |

Bold entries are represent wild-type contact residues and do not constitute “modifications” as used herein.
An asterisk indicates that the residue contacts the base on the antisense strand.

2.2.3 Rationally-Designed Meganucleases Derived from I-SceI

In another aspect, the present invention relates to non-naturally-occurring, rationally-designed meganucleases which are based upon or derived from the I-SceI meganuclease of Saccharomyces cerevisiae. The wild-type amino acid sequence of the I-SceI meganuclease is shown in SEQ ID NO: 9, which corresponds to Genbank Accession #CAA09843. The recognition sequence of the wild-type I-SceI meganuclease from crystal structure having PDB identifier (PDB ID) 1R7M is shown below:

| Sense | 5′-T T A C C C T G T T A T C C C T A G-3′ SEQ ID NO: 10 |
|---|---|
| Antisense | 3′-A A T G G G A C A A T A G G G A T C-5′ SEQ ID NO: 11 |
| Position | 1 2 3 4 5 6 7 8 9 10 11 12 13 14 15 16 17 18 |

Note that the recognition sequence is non-palindromic and there are not four base pairs separating half-sites.

In accordance with the invention, a substantial number of amino acid modifications to the DNA recognition domain of the I-SceI meganuclease have been identified which, singly or in combination, can result in recombinant meganucleases with specificities altered at individual bases within the DNA recognition sequence, such that these non-naturally-occurring, rationally-designed meganucleases have recognition sequences different from the wild-type enzyme. The amino acid modifications of I-SceI and the predicted change in recognition sequence specificity are shown in Table 3:

TABLE 3

Favored Sense-Strand Base

| Position | A | C | G | T |
|---|---|---|---|---|
| 4 | K50 | R50* | E50* | K57 |
| | | K50* | R57 | M57 |
| | | E57 | K57 | Q50* |
| 5 | K48 | R48* | E48* | Q48* |
| | Q102 | K48* | K102 | C102 |
| | | E102 | R102 | L102 |
| | | E59 | | V102 |
| 6 | K59 | R59* | K84 | Q59* |
| | | K59* | E59* | Y46 |
| 7 | C46* | R46* | K86 | K68 |
| | L46* | K46* | R86 | C86 |
| | V46* | E86 | E46* | L86 |
| | | | | Q46* |
| 8 | K61* | E88 | E61* | K88 |
| | S61* | R61* | R88 | Q61* |
| | V61* | H61* | K88 | H61* |
| | A61* | | | |
| | L61* | | | |
| 9 | T98* | R98* | E98* | Q98* |
| | C98* | K98* | D98* | |
| | V98* | | | |
| | L98* | | | |
| 10 | V96* | K96* | D96* | Q96* |
| | C96* | R96* | E96* | |
| | A96* | | | |
| 11 | C90* | K90* | E90* | Q90* |
| | L90* | R90* | | |
| 12 | Q193 | E165 | K165 | C165 |
| | | E193 | R165 | L165 |
| | | D193 | | C193 |
| | | | | V193 |
| | | | | A193 |
| | | | | T193 |
| | | | | S193 |
| 13 | C193* | K193* | E193* | Q193* |
| | L193* | R193* | D193* | C163 |
| | | D192 | K163 | L163 |
| | | | R192 | |
| 14 | L192* | E161 | K147 | K161 |
| | C192* | R192* | K161 | Q192* |
| | | K192* | R161 | |
| | | | R197 | |
| | | | D192* | |
| | | | E192* | |
| 15 | | E151 | K151 | C151 |
| | | | | L151 |
| | | | | K151 |
| 17 | N152* | K152* | N152* | Q152* |
| | S152* | K150* | S152* | Q150* |
| | C150* | | D152* | |
| | L150* | | D150* | |
| | V150* | | E150* | |
| | T150* | | | |
| 18 | K155* | R155* | E155* | H155* |
| | C155* | K155* | | Y155* |

Bold entries are wild-type contact residues and do not constitute “modifications” as used herein.
An asterisk indicates that the residue contacts the base on the antisense strand.

2.2.4 Rationally-Designed Meganucleases Derived from I-CeuI

In another aspect, the present invention relates to non-naturally-occurring, rationally-designed meganucleases which are based upon or derived from the I-CeuI meganuclease of Chlamydomonas eugametos. The wild-type amino acid sequence of the I-CeuI meganuclease is shown in SEQ ID NO: 12, which corresponds to Genbank Accession #P32761. Two recognition sequence half sites of the wild-type I-CeuI meganuclease from crystal structure having PDB identifier (PDB ID) 2EX5 are shown below:

| | |
|---|---|
| Position | -9-8-7-6-5-4-3-2-1 |
| | 5′-A T A A C G G T C C T A A G G T A G C G A A-3′ SEQ ID NO: 13 |
| | 3′-T A T T G C C A G G A T T C C A T C G C T T-5′ SEQ ID NO: 14 |
| Position | -1-2-3-4-5-6-7-8-9 |

Note that the recognition sequence is non-palindromic, even outside the central four base pairs, despite the fact that I-CeuI is a homodimer, due to the natural degeneracy in the I-CeuI recognition interface (Spiegel et al. (2006), Structure 14:869-80). The two recognition sequence half-sites are shown in bold on their respective sense strands.

In accordance with the invention, a substantial number of amino acid modifications to the DNA recognition domain of the I-CeuI meganuclease have been identified which, singly or in combination, result in recombinant meganucleases with specificities altered at individual bases within the DNA recognition sequence half-site, such that these non-naturally-occurring, rationally-designed meganucleases can have recognition sequences different from the wild-type enzyme. The amino acid modifications of I-CeuI and the predicted change in recognition sequence specificity are shown in Table 4:

TABLE 4

Favored Sense-Strand Base

| Position | A | C | G | T |
|---|---|---|---|---|
| −1 | C92* | K116* | E116* | Q116* |
| | A92* | R116* | E92* | Q92* |
| | V92* | D116* | | |
| | | K92* | | |
| −2 | Q117 | E117 | K117 | C117 |
| | C90* | D117 | R124 | V117 |
| | L90* | R174* | K124 | T117 |
| | V90* | K124* | E124* | Q90* |
| | | K90* | E90* | |
| | | R90* | D90* | |
| | | K68* | | |
| −3 | C70* | K70* | E70* | Q70* |
| | V70* | | E88* | |
| | T70* | | | |
| | L70* | | | |
| | K70* | | | |
| −4 | Q126 | E126 | R126 | K126 |
| | N126 | D126 | K126 | L126 |
| | K88* | R88* | E88* | Q88* |
| | L88* | K88* | D88* | |
| | C88* | K72* | | |
| | C72* | | | |
| | L72* | | | |
| | V72* | | | |
| −5 | C74* | K74* | E74* | C128 |
| | L74* | | K128 | L128 |
| | V74* | | R128 | V128 |
| | T74* | | E128 | T128 |
| −6 | Q86 | D86 | K128 | K86 |
| | | E86 | R128 | C86 |
| | | R84* | R86 | L86 |
| | | K84* | K86 | |
| | | | E84* | |
| −7 | L76* | R76* | E76* | H76* |
| | C76* | K76* | R84 | Q76* |
| | K76* | H76* | | |
| −8 | Y79 | D79 | R79 | C79 |
| | R79 | E79 | K79 | L79 |
| | Q76 | D76 | K76 | V79 |
| | | E76 | R76 | L76 |
| −9 | Q78 | D78 | R78 | K78 |
| | N78 | E78 | K78 | V78 |
| | H78 | | H78 | L78 |
| | K78 | | | C78 |
| | | | | T78 |

Bold entries are wild-type contact residues and do not constitute “modifications” as used herein.
An asterisk indicates that the residue contacts the base on the antisense strand.

### 2.2.5 Optionally-Excluded Recombinant Meganucleases

In some embodiments, the present invention is not intended to embrace certain recombinant meganucleases which have been described in the prior art, and which have been developed by alternative methods. These excluded meganucleases include those described by Arnould et al. (2006), J. Mol. Biol. 355:443-58; Sussman et al. (2004), J. Mol. Biol. 342:31-41; Chames et al. (2005), Nucleic Acids Res. 33: e178; Seligman et al. (2002), Nucleic Acids Res. 30:3870-9; and Ashworth et al. (2006), Nature 441 (7093): 656-659; the entire disclosures of which are hereby incorporated by reference, including recombinant meganucleases based on I-CreI with single substitutions selected from C33, R33, A44, H33, K32, F33, R32, A28, A70, E33, V33, A26, and R66. Also excluded are recombinant meganucleases based on I-CreI with three substitutions selected from A68/N70/N75 and D44/D70/N75, or with four substitutions selected from K44/T68/G60/N75 and R44/A68/T70/N75. Lastly, specifically excluded is the recombinant meganuclease based on I-MsoI with the pair of substitutions L28 and R83. These substitutions or combinations of substitutions are referred to herein as the “excluded modifications.”

2.2.6 Rationally-Designed Meganucleases with Multiple Changes in the Recognition Sequence Half-Site

In another aspect, the present invention relates to non-naturally-occurring, rationally-designed meganucleases which are produced by combining two or more amino acid modifications as described in sections 2.2.1-2.2.4 above, in order to alter half-site preference at two or more positions in a DNA recognition sequence half-site. For example, without limitation, and as more fully described below, the enzyme DJ1 was derived from I-CreI by incorporating the modifications R30/E38 (which favor C at position −7), R40 (which favors G at position −6), R42 (which favors at G at position −5), and N32 (which favors complete degeneracy at position −9). The rationally-designed DJ1 meganuclease invariantly recognizes C₋₇ G₋₆ G₋₅ compared to the wild-type preference for A₋₇ A₋₆ C₋₅, and has increased tolerance for A at position −9.

The ability to combine residue substitutions that affect different base positions is due in part to the modular nature of the LAGLIDADG meganucleases. A majority of the base contacts in the LAGLIDADG recognition interfaces are made by individual amino acid side chains, and the interface is relatively free of interconnectivity or hydrogen bonding networks between side chains that interact with adjacent bases. This generally allows manipulation of residues that interact with one base position without affecting side chain interactions at adjacent bases. The additive nature of the mutations listed in sections 2.2.1-2.2.4 above is also a direct result of the method used to identify these mutations. The method predicts side chain substitutions that interact directly with a single base. Interconnectivity or hydrogen bonding networks between side chains is generally avoided to maintain the independence of the substitutions within the recognition interface.

Certain combinations of side chain substitutions are completely or partially incompatible with one another. When an incompatible pair or set of amino acids are incorporated into a rationally-designed meganuclease, the resulting enzyme will have reduced or eliminated catalytic activity. Typically, these incompatibilities are due to steric interference between the side chains of the introduced amino acids and activity can be restored by identifying and removing this interference. Specifically, when two amino acids with large side chains (e.g., amino acids from group 2 or 3) are incorporated at amino acid positions that are adjacent to one another in the meganuclease structure (e.g., positions 32 and 33, 28 and 40, 28 and 42, 42 and 77, or 68 and 77 in the case of meganucleases derived from I-CreI), it is likely that these two amino acids will interfere with one another and reduce enzyme activity. This interference be eliminated by substituting one or both incompatible amino acids to an amino acid with a smaller side chain (e.g., group 1 or group 2). For example, in rationally-designed meganucleases derived from I-CreI, K28 interferes with both R40 and R42. To maximize enzyme activity, R40 and R42 can be combined with a serine or aspartic acid at position 28.

Combinations of amino substitutions, identified as described herein, can be used to rationally alter the specificity of a wild-type meganuclease (or a previously modified meganuclease) from an original recognition sequence to a desired recognition sequence which may be present in a nucleic acid of interest (e.g., a genome). FIG. 2A, for example, shows the “sense” strand of the I-CreI meganuclease recognition sequence WT (SEQ ID NO: 4) as well as a number of other sequences for which a rationally-designed meganuclease would be useful. Conserved bases between the WT recognition sequence and the desired recognition sequence are shaded. In accordance with the invention, recombinant meganucleases based on the I-CreI meganuclease can be rationally-designed for each of these desired recognition sequences, as well as any others, by suitable amino acid substitutions as described herein.

### 3. Rationally-Designed Meganucleases with Altered DNA-Binding Affinity

As described above, the DNA-binding affinity of the recombinant meganucleases described herein can be modulated by altering certain amino acids that form the contact surface with the phosphodiester backbone of DNA. The contact surface comprises those amino acids in the enzyme with β-carbons less than 9 Å from the DNA backbone, and with side chains oriented toward the DNA, irrespective of whether the residues make contacts with the DNA backbone in the wild-type meganuclease-DNA complex. Because DNA-binding is a necessary precursor to enzyme activity, increases/decreases in DNA-binding affinity have been shown to cause increases/decreases, respectively, in enzyme activity. However, increases/decreases in DNA-binding affinity also have been shown to cause decreases/increases in the meganuclease sequence-specificity. Therefore, both activity and specificity can be modulated by modifying the phosphodiester backbone contacts.

Specifically, to increase enzyme activity/decrease enzyme specificity:
(i) Remove electrostatic repulsion between the enzyme and DNA backbone. If an identified amino acid has a negatively-charged side chain (e.g., aspartic acid, glutamic acid) which would be expected to repulse the negatively-charged DNA backbone, the repulsion can be eliminated by substituting an amino acid with an uncharged or positively-charged side chain, subject to effects of steric interference. An experimentally verified example is the mutation of glutamic acid 80 in I-CreI to glutamine.
(ii) Introduce electrostatic attraction interaction between the enzyme and the DNA backbone. At any of the positions of the contact surface, the introduction of an amino acid with a positively-charged side chain (e.g., lysine or arginine) is expected to increase binding affinity, subject to effects of steric interference.
(iii) Introduce a hydrogen-bond between the enzyme and the DNA backbone. If an amino acid of the contact surface does not make a hydrogen bond with the DNA backbone because it lacks an appropriate hydrogen-bonding functionality or has a side chain that is too short, too long, and/or too inflexible to interact with the DNA backbone, a polar amino acid capable of donating a hydrogen bond (e.g., serine, threonine, tyrosine, histidine, glutamine, asparagine, lysine, cysteine, or arginine) with the appropriate length and flexibility can be introduced, subject to effects of steric interference.

Specifically, to decrease enzyme activity/increase enzyme specificity:
(i) Introduce electrostatic repulsion between the enzyme and the DNA backbone. At any of the positions of the contact surface, the introduction of an amino acid with a negatively-charged side chain (e.g., glutamic acid, aspartic acid) is expected to decrease binding affinity, subject to effects of steric interference.
(ii) Remove electrostatic attraction between the enzyme and DNA. If any amino acid of the contact surface has a positively-charged side chain (e.g., lysine or arginine) that interacts with the negatively-charged DNA backbone, this favorable interaction can be eliminated by substituting an amino acid with an uncharged or negatively-charged side chain, subject to effects of steric interference. An experimentally verified example is the mutation of lysine 116 in I-CreI to aspartic acid.
(iii) Remove a hydrogen-bond between the enzyme and the DNA backbone. If any amino acid of the contact surface makes a hydrogen bond with the DNA backbone, it can be substituted to an amino acid that would not be expected to make a similar hydrogen bond because its side chain is not appropriately functionalized or it lacks the necessary length/flexibility characteristics.

For example, in some recombinant meganucleases based on I-CreI, the glutamic acid at position 80 in the I-CreI meganuclease is altered to either a lysine or a glutamine to increase activity. In another embodiment, the tyrosine at position 66 of I-CreI is changed to arginine or lysine, which increases the activity of the meganuclease. In yet another embodiment, enzyme activity is decreased by changing the lysine at position 34 of I-CreI to aspartic acid, changing the tyrosine at position 66 to aspartic acid, and/or changing the lysine at position 116 to aspartic acid.

The activities of the recombinant meganucleases can be modulated such that the recombinant enzyme has anywhere from no activity to very high activity with respect to a particular recognition sequence. For example, the DJ1 recombinant meganuclease when carrying glutamic acid mutation at position 26 loses activity completely. However, the combination of the glutamic acid substitution at position 26 and a glutamine substitution at position 80 creates a recombinant meganuclease with high specificity and activity toward a guanine at −4 within the recognition sequence half-site (see FIG. 1(D)).

In accordance with the invention, amino acids at various positions in proximity to the phosphodiester DNA backbone can be changed to simultaneously affect both meganuclease activity and specificity. This “tuning” of the enzyme specificity and activity is accomplished by increasing or decreasing the number of contacts made by amino acids with the phosphodiester backbone. A variety of contacts with the phosphodiester backbone can be facilitated by amino acid side chains. In some embodiments, ionic bonds, salt bridges, hydrogen bonds, and steric hindrance affect the association of amino acid side chains with the phosphodiester backbone. For example, for the I-CreI meganuclease, alteration of the lysine at position 116 to an aspartic acid removes a salt bridge between nucleic acid base pairs at positions −8 and −9, reducing the rate of enzyme cleavage but increasing the specificity.

The residues forming the backbone contact surface of each of the wild-type I-CreI (SEQ ID NO: 1), I-MsoI (SEQ ID NO: 6), I-SceI (SEQ ID NO: 9) and I-CeuI (SEQ ID NO: 12) meganucleases are identified in Table 5 below:

TABLE 5

| I-CreI | I-MsoI | I-SceI | I-CeuI |
|---|---|---|---|
| P29, K34, T46, | K36, Q41, R51, | N15, N17, L19, | K21, D25, |
| K48, R51, V64, | N70, I85, G86, | K20, K23, K63, | K28, K31, |
| Y66, E80, I81, | S87, T88, H89, | L80, S81, H84, | S68, N70, |
| K82, L112, | Y118, Q122, | L92, N94, N120, | H94, R112, |
| K116, D137, | K123, Q139, | K122, K148, | R114, S117, |
| K139, T140, | K143, R144, | Y151, K153, | N120, D128, |
| T143 | E147, S150, | T156, N157, | N129, R130, |
| | N152 | S159, N163, | H172 |
| | | Q165, S166, | |
| | | Y188, K190, | |
| | | I191, K193, | |
| | | N194, K195, | |
| | | Y199, D201, | |
| | | S202, Y222, | |
| | | K223 | |

To increase the affinity of an enzyme and thereby make it more active/less specific:
(1) Select an amino acid from Table 5 for the corresponding enzyme that is either negatively-charged (D or E), hydrophobic (A, C, F, G, I, L, M, P, V, W, Y), or uncharged/polar (H, N, Q, S, T).
(2) If the amino acid is negatively-charged or hydrophobic, mutate it to uncharged/polar (less effect) or positively-charged (K or R, more effect).
(3) If the amino acid is uncharged/polar, mutate it to positively-charged.

To decrease the affinity of an enzyme and thereby make it less active/more specific:
(1) Select an amino acid from Table 5 for the corresponding enzyme that is either positively-charged (K or R), hydrophobic (A, C, F, G, I, L, M, P, V, W, Y), or uncharged/polar (H, N, Q, S, T).
(2) If the amino acid is positively-charged, mutate it to uncharged/polar (less effect) or negatively-charged (more effect).
(3) If the amino acid is hydrophobic or uncharged/polar, mutate it to negatively-charged.

### 4. Rationally-Designed Heterodimeric Meganucleases

In another aspect, the invention provides rationally-designed, non-naturally-occurring meganucleases which are heterodimers formed by the association of two monomers, one of which may be a wild-type and one or both of which may be a non-naturally-occurring or recombinant form. For example, wild-type I-CreI meganuclease is normally a homodimer composed of two monomers that each bind to one half-site in the pseudo-palindromic recognition sequence. A heterodimeric recombinant meganuclease can be produced by combining two meganucleases that recognize different half-sites, for example by co-expressing the two meganucleases in a cell or by mixing two meganucleases in solution. The formation of heterodimers can be favored over the formation of homodimers by altering amino acids on each of the two monomers that affect their association into dimers. In particular embodiments, certain amino acids at the interface of the two monomers are altered from negatively-charged amino acids (D or E) to positively charged amino acids (K or R) on a first monomer and from positively charged amino acids to negatively-charged amino acids on a second monomer (Table 6). For example, in the case of meganucleases derived from I-CreI, lysines at positions 7 and 57 are mutated to glutamic acids in the first monomer and glutamic acids at positions 8 and 61 are mutated to lysines in the second monomer. The result of this process is a pair of monomers in which the first monomer has an excess of positively-charged residues at the dimer interface and the second monomer has an excess of negatively-charged residues at the dimer interface. The first and second monomer will, therefore, associate preferentially over their identical monomer pairs due to the electrostatic interactions between the altered amino acids at the interface.

TABLE 6

| I-CreI: First Monomer | I-CreI: Second Monomer |
|---|---|
| Substitutions | Substitutions |
| K7 to E7 or D7 | E8 to K8 or R8 |
| K57 to E57 or D57 | E61 to K61 or R61 |
| K96 to E96 or D96 | |
| I-MsoI: First Monomer | I-MsoI: Second Monomer |
| Substitutions | Substitutions |
| R302 to E302 or D302 | D20 to K60 or R60 |
| | E11 to K11 or R11 |
| | Q64 to K64 or R64 |
| I-CeuI: First Monomer | I-CeuI: Second Monomer |
| Substitutions | Substitutions |
| R93 to E93 or D93 | E152 to K152 or R152 |

Alternatively, or in addition, certain amino acids at the interface of the two monomers can be altered to sterically hinder homodimer formation. Specifically, amino acids in the dimer interface of one monomer are substituted with larger or bulkier residues that will sterically prevent the homodimer. Amino acids in the dimer interface of the second monomer optionally can be substituted with smaller residues to compensate for the bulkier residues in the first monomer and remove any clashes in the heterodimer, or can be unmodified.

In another alternative or additional embodiment, an ionic bridge or hydrogen bond can be buried in the hydrophobic core of a heterodimeric interface. Specifically, a hydrophobic residue on one monomer at the core of the interface can be substituted with a positively charged residue. In addition, a hydrophobic residue on the second monomer, that interacts in the wild type homodimer with the hydrophobic residue substituted in the first monomer, can be substituted with a negatively charged residue. Thus, the two substituted residues can form an ionic bridge or hydrogen bond. At the same time, the electrostatic repulsion of an unsatisfied charge buried in a hydrophobic interface should disfavor homodimer formation.

Finally, as noted above, each monomer of the heterodimer can have different amino acids substituted in the DNA recognition region such that each has a different DNA half-site and the combined dimeric DNA recognition sequence is non-palindromic.

### 5. Rationally-Designed Inactive Meganucleases as Meganuclease DNA-Binding Domains

The catalytic activity of a non-naturally-occurring, rationally-designed meganuclease can be reduced or eliminated by mutating amino acids involved in catalysis (e.g., the mutation of Q47 to E in I-CreI, see Chevalier et al. (2001), Biochemistry. 43:14015-14026); the mutation of D44 or D145 to N in I-SceI; the mutation of E66 to Q in I-CeuI; the mutation of D22 to N in I-MsoI). The inactivated meganuclease can then be fused to an effector domain from another protein including, but not limited to, a transcription activator (e.g., the GAL4 transactivation domain or the VP16 transactivation domain), a transcription repressor (e.g., the KRAB domain from the Kruppel protein), a DNA methylase domain (e.g., M.CviPI or M.SssI), or a histone acetyltransferase domain (e.g., HDAC1 or HDAC2). Chimeric proteins consisting of an engineered DNA-binding domain, most notably an engineered zinc finger domain, and an effector domain are known in the art (see, e.g., Papworth et al. (2006), Gene 366:27-38).

In some embodiments, the meganuclease will also comprise a nuclear localization signal (e.g. the SV40 NLS (SEQ ID NO. 38), which can be added to the N-terminus of the meganuclease domain). The meganuclease DNA-binding domain may comprise a mono-LAGLIDADG meganuclease domain which recognizes a palindromic or pseudo-palindromic DNA sequence. Alternatively, it may comprise a di-LAGLIDADG meganuclease domain or a mono-LAGLIDADG meganuclease domain which can form a heterodimer, regardless of whether or not the mono-LAGLIDADG domain has been engineered to force heterodimerization, which can bind to a non-palindromic DNA sequence. Lastly, the meganuclease DNA-binding domain may comprise a single-chain meganuclease in which a pair of mono-LAGLIDADG subunits derived from I-CreI are joined into a single polypeptide. The latter embodiment is useful for the recognition of non-palindromic DNA sites.

### 6. Recognition Sites for Meganuclease DNA-Binding Domains

To influence the expression of a gene of interest, the engineered meganuclease DNA-binding domain (“meganuclease DNA-binding domain”) can recognize a DNA site in the gene or in the gene promoter. If the goal is gene activation, the meganuclease DNA-binding domain can recognize a DNA site in the promoter that is upstream from the start of gene transcription. If the goal is gene repression, the meganuclease DNA-binding domain can recognize a DNA site which is upstream or downstream from the transcription start site in either the promoter of the gene itself. In some embodiments, the meganuclease DNA-binding domain will recognize a DNA site that is within 2,000 bases of the transcription start site. In some embodiments, the meganuclease DNA-binding domain will recognize a DNA site that is within 500 bases of the transcription start site. In the case of a meganuclease DNA-binding domain intended to repress gene expression, it may be useful if the meganuclease DNA-binding domain recognizes a DNA site which is as close to the transcription start site as possible.

The transcription start sites of many genes of interest are known in the art and can be readily found in the scientific literature or in databases such as GenBank (http://www.ncbi.nlm.nih.gov/Genbank/). Alternatively, the transcription start site for a gene of interest may be determined experimentally by RT-PCR or other methods that are known in the art (see, e.g., Ohara, et al. (1990), Nuc. Acids Res. 23:6997-7002).

In some embodiments, where the intent of a targeted transcriptional effector is to control the expression of a native gene in a eukaryotic cell, the meganuclease DNA-binding domain can be designed to bind a recognition sequence which is known in advance to be in an accessible region of the chromatin. The accessibility of a particular recognition sequence can be determined by DNaseI hypersensitivity analysis. Such analyses have been performed for many genes of interest and are well-known in the scientific literature. In cases where such data are not already publicly available, DNaseI sensitivity may be determined experimentally using standard protocols (e.g., Lu and Richardson (2004), Methods Mol. Biol. 287:77-86). Alternatively, a meganuclease DNA-binding domain may be produced that binds to a recognition sequence in or near the recognition sequence for a known, native transcription factor. The DNA sequences recognized by many native transcription factors are known in the art (see, e.g., the TRANSFAC database, www.gene-regulation.com). Where such DNA sequences appear in the promoters of genes, it is generally believed that those sites, as well as the immediately flanking regions, are accessible within the chromatin structure.

Several methods exist to determine whether or not a meganuclease DNA-binding domain derived from an rationally-designed meganuclease binds to a particular DNA sequence. Methods for determining DNA-binding affinity in vitro are known in the art and include techniques such as electrophoretic mobility shift assay (EMSA; see, e.g., Ausubel et al. (1999), Curr. Protoc. Mol. Biol.). In addition, it is possible to use common experimental techniques such as chromatin immunoprecipitation to determine whether or not a particular meganuclease DNA-binding domain binds to a specific DNA sequence in vivo (see, e.g., Aparicio et al. (2005), Curr. Protoc. Mol. Biol. 21:21-3; see also Example 5).

### 7. Transcription Effector Domains

A transcription effector domain will affect gene expression by interacting, directly or indirectly, with the cellular transcription machinery. Effector domains can be found as part of natural transcription factors and are distinguished by their ability to either activate or repress gene transcription. Many transcription activator domains are known in the art and include the GAL4 activation domain (comprising amino acids 768-881 of the S. cerevisiae GAL4 protein, SEQ ID NO: 39) and the Herpes virus VP16 activation domain (comprising amino acids 413-490 of the HSV-1 VP16 protein, SEQ ID NO: 40). Transcription repressor domains are also known in the art and include the KRAB (Kruppel Associated Box) family of repressor domains. KRAB domains are ubiquitous in nature where they are typically found as components of Cys₂His₂ zinc finger transcription factors (see, e.g., Huntley et al. (2006), Genome Res. 16:669-677). For example, one KRAB domain suitable for some embodiments of the invention comprises amino acids 12-74 of the Rattus norvegicus Kid-1 protein (GenBank accession number Q02975, SEQ ID NO: 41).

Transcription effector domains may be fused to either the N- or C-terminus of a meganuclease-derived DNA-binding domain. In the case of meganuclease DNA-binding domains derived from I-CreI, it may be preferable to fuse the effector domain to the C-terminus. In addition, it may be preferable to add a short, flexible amino acid “domain linker” between the DNA-binding domain and the effector domain. Suitable embodiments include linkers of the form (Gly-Ser-Ser) n wherein n=1-5. The use of flexible linkers rich in glycine and serine amino acids to join protein domains is known in the art (e.g., Mack et al. (1995), Proc. Nat. Acad. Sci. USA 92:7021-7025; Ueda et al. (2000), J. Immunol. Methods 241:159-170; Brodelius et al. (2002), 269:3570-3577; Kim et al. (1996), Proc. Nat. Acad. Sci. USA 93:1156-1160). Domain linkers other than short, flexible amino acid linkers can, as described above, also be used.

### 8. Regulation of Transcription

Targeted transcriptional effectors described herein can be used to control gene expression in isolated cells or organisms. For most applications, a targeted transcriptional effector will be produced to bind to and regulate a native promoter/gene in a prokaryotic or eukaryotic cell. In some cases, however, it may be desirable to produce a targeted transcriptional effector which binds to and regulates an exogenous promoter/gene that has been introduced into the cell. Such an exogenous promoter/gene could exist in the cell extrachromosomally (e.g., on a plasmid) or it could be integrated into the genome of the cell (e.g., by viral transduction). In some embodiments, a targeted transcriptional effector may be produced to bind and regulate the genes of a virus (e.g. HIV or HSV-1) such that the pathogenicity of the virus is reduced. For example, a targeted transcriptional effector may be used to reduce the expression of viral genes necessary for integration into the host genome, replication, the emergence from latency, virus particle formation, cell exit, or the evasion of host defenses.

Targeted transcriptional effectors can be delivered to cells as protein or in the form of a nucleic acid which encodes the protein. In general, the effects that a targeted transcriptional effector exert on the expression of a gene of interest will persist only as long as the targeted transcriptional effector itself exists within the cell. Thus, delivery of a targeted transcriptional effector in protein form can be expected to yield a transient effect on gene transcription (e.g., a few days). Delivery of a targeted transcriptional effector gene carried on a non-replicating nucleic acid (e.g., non-replicating plasmid DNA) to a cell can be expected to effect the transcription of the gene of interest for a longer period of time (e.g., days to weeks). Delivery of a targeted transcriptional effector gene carried on a replicating nucleic acid (e.g., a replicating plasmid or a virus that integrates into the genome) can be expected to effect the expression of a gene of interest for the greatest length of time and can be made permanent.

The present disclosure provides targeted transcriptional effectors that have been engineered to specifically recognize, with high efficacy, endogenous cellular genes. Thus, the present disclosure demonstrates that targeted transcriptional effectors based on engineered meganucleases can be used to regulate expression of an endogenous cellular gene that is present in its native chromatin environment.

In some embodiments, the methods of regulation use targeted transcriptional effectors with a K_{d} for the targeted recognition sequence of less than about 25 nM to activate or repress gene transcription. The targeted transcriptional repressors can be used to decrease transcription of an endogenous cellular gene by 20% or more, and targeted transcriptional activators can be used to increase transcription of an endogenous cellular gene by 20% or more (as measured by changes in transcript number during the first half-life of the targeted transcriptional effector after administration).

### 9. Applications of Targeted Transcriptional Effectors

The methods described herein for regulating gene expression allow for novel human and mammalian therapeutic applications, e.g., treatment of genetic diseases; cancer; fungal, protozoal, bacterial, and viral infection; ischemia; vascular disease; arthritis; immunological disorders; etc., as well as providing means for functional genomics assays, and means for developing plants with altered phenotypes, including disease resistance, fruit ripening, sugar and oil composition, yield, and color.

As described herein, targeted transcriptional activators can be designed to recognize any suitable target site, for regulation of expression of any endogenous gene of choice. Examples of endogenous genes suitable for regulation include VEGF, CCR5, ERα, Her2/Neu, Tat, Rev, HBV C, S, X, and P, LDL-R, PEPCK, CYP7, Fibrinogen, ApoB, Apo E, Apo(a), renin, NF-κB, I-κB, TNF-α, FAS ligand, amyloid precursor protein, atrial naturetic factor, ob-leptin, ucp-1, IL-1, IL-2, IL-3, IL-4, IL-5, IL-6, IL-12, G-CSF, GM-CSF, Epo, PDGF, PAF, p53, Rb, fetal hemoglobin, dystrophin, eutrophin, GDNF, NGF, IGF-1, VEGF receptors flt and flk, topoisomerase, telomerase, bcl-2, cyclins, angiostatin, IGF, ICAM-1, STATS, c-myc, c-myb, TH, PTI-1, polygalacturonase, EPSP synthase, FAD2-1, delta-12 desaturase, delta-9 desaturase, delta-15 desaturase, acetyl-CoA carboxylase, acyl-ACP-thioesterase, ADP-glucose pyrophosphorylase, starch synthase, cellulose synthase, sucrose synthase, senescence-associated genes, heavy metal chelators, fatty acid hydroperoxide lyase, viral genes, protozoal genes, fungal genes, and bacterial genes. In general, suitable genes to be regulated include cytokines, lymphokines, growth factors, mitogenic factors, chemotactic factors, onco-active factors, receptors, potassium channels, G-proteins, signal transduction molecules, and other disease-related genes.

A general theme in transcription factor regulation of gene expression is that simple binding and sufficient proximity to the promoter are all that is generally needed. Exact positioning relative to the promoter, orientation and, within limits, distance do not matter greatly. This feature allows considerable flexibility in choosing sites for constructing artificial transcription factors. Therefore, the target site recognized by the targeted transcriptional effector can be any suitable site in the target gene that will allow activation or repression of gene expression by a targeted transcriptional effector, optionally linked to a regulatory domain. Possible target sites include regions adjacent to, downstream, or upstream of the transcription start site. In addition, target sites that are located in enhancer regions, repressor sites, RNA polymerase pause sites, and specific regulatory sites (e.g., SP-1 sites, hypoxia response elements, nuclear receptor recognition elements, p53 binding sites), sites in the cDNA encoding region or in an expressed sequence tag (EST) coding region.

In another embodiment, the targeted transcriptional activator is linked to at least one or more regulatory domains, described below. Examples of regulatory domains include transcription factor repressor or activator domains such as KRAB and VP16, co-repressor and co-activator domains, DNA methyl transferases, histone acetyltransferases, histone deacetylases, and endonucleases such as Fokl. For repression of gene expression, typically the expression of the gene is reduced by about 20% (i.e., 80% of non-targeted transcriptional activator modulated expression), about 50% (i.e., 50% of non-targeted transcriptional activator modulated expression), or about 75-100% (i.e., 25% to 0% of non-targeted transcriptional activator modulated expression). For activation of gene expression, typically expression is activated by about 20% (i.e., 120% of non-targeted transcriptional activator modulated expression), about 50% (i.e., 150% of non-targeted transcriptional activator modulated expression), about 100% (i.e., 200% of non-targeted transcriptional activator modulated expression), about 5-10 fold (i.e., 500-1000% of non-targeted transcriptional activators modulated expression), up to at least 100 fold or more.

The expression of targeted transcriptional effectors (activators and repressors) can also be controlled by systems typified by the tet-regulated systems and the RU-486 system (see, e.g., Gossen & Bujard (1992), Proc. Natl. Acad. Sci. USA 89:5547; Oligino et al. (1998), Gene Ther. 5:491-496; Wang et al. (1997), Gene Ther. 4:432-441; Neering et al. (1996), Blood 88:1147-1155; and Rendahl et al. (1998), Nat. Biotechnol. 16:757-761). These impart small molecule control on the expression of the targeted transcriptional effector activators and repressors and thus impart small molecule control on the target gene(s) of interest. This beneficial feature could be used in cell culture models, in gene therapy, and in transgenic animals and plants.

The practice of conventional techniques in molecular biology, biochemistry, chromatin structure and analysis, computational chemistry, cell culture, recombinant DNA, bioinformatics, genomics and related fields are well-known to those of skill in the art and are discussed, for example, in the following literature references: Sambrook et al., Molecular Cloning: A Laboratory Manual, Second edition, Cold Spring Harbor Laboratory Press, 1989; Ausubel et al., Current Protocols In Molecular Biology, John Wiley & Sons, New York, 1987 and periodic updates; the series Methods In Enzymology, Academic Press, San Diego; Wolffe, Chromatin Structure And Function, Third edition, Academic Press, San Diego, 1998; Methods In Enzymology, Vol. 304, “Chromatin” (P. M. Wassarman and A. P. Wolffe, eds.), Academic Press, San Diego, 1999; and Methods In Molecular Biology, Vol. 119, “Chromatin Protocols” (P. B. Becker, ed.) Humana Press, Totowa, 1999, all of which are incorporated by reference in their entireties.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

Further, a promoter can be a normal cellular promoter or, for example, a promoter of an infecting microorganism such as, for example, a bacterium or a virus. For example, the long terminal repeat (LTR) of retroviruses is a promoter region which may be a target for a modified zinc finger binding polypeptide. Promoters from members of the Lentivirus group, which include such pathogens as human T-cell lymphotrophic virus (HTLV) 1 and 2, or human immunodeficiency virus (HIV) 1 or 2, are examples of viral promoter regions which may be targeted for transcriptional modulation by a modified zinc finger binding polypeptide as described herein.

To determine the level of gene expression modulation by a targeted transcriptional effector, cells contacted with targeted transcriptional effectors are compared to control cells, e.g., without the targeted transcriptional effector, to examine the extent of inhibition or activation. Control samples are assigned a relative gene expression activity value of 100%.

A “promoter” is defined as an array of nucleic acid control sequences that direct transcription. As used herein, a promoter typically includes necessary nucleic acid sequences near the start site of transcription, such as, in the case of certain RNA polymerase II type promoters, a TATA element, enhancer, CCAAT box, SP-1 site, etc.

As used herein, a promoter also optionally includes distal enhancer or repressor elements, which can be located as much as several thousand base pairs from the start site of transcription. The promoters often have an element that is responsive to transactivation by a DNA-binding moiety such as a polypeptide, e.g., a nuclear receptor, Gal4, the lac repressor and the like.

A “transcriptional activator” and a “transcriptional repressor” refer to proteins or functional fragments of proteins that have the ability to modulate transcription. Such proteins include, e.g., transcription factors and co-factors (e.g., KRAB, MAD, ERD, SID, nuclear factor kappa B subunit p65, early growth response factor 1, and nuclear hormone receptors, VP16, VP64), endonucleases, integrases, recombinases, methyltransferases, histone acetyltransferases, histone deacetylases etc.

Activators and repressors include co-activators and co-repressors (see, e.g., Utley et al. (1998), Nature 394:498-502).

A “fusion molecule” is a molecule in which two or more subunit molecules are physically joined or linked (e.g., covalently). The subunit molecules can be the same chemical type of molecule, or can be different chemical types of molecules. Examples of the first type of fusion molecule include, but are not limited to, fusion polypeptides (for example, a fusion between an engineered meganuclease DNA-binding domain and a transcriptional effector domain) and fusion nucleic acids (for example, a nucleic acid encoding the fusion polypeptide described herein). An example of the second type of fusion molecule includes, but is not limited to, a fusion between a DNA-binding protein and a nucleic acid.

### 10. Targeted Transcriptional Effectors Comprising a Regulatory Domain

In some embodiments, the invention provides a targeted transcriptional effector comprising: (i) an engineered meganuclease DNA-binding domain lacking endonuclease cleavage activity that is engineered to bind to a target site in a gene of interest; and (ii) a regulatory domain, wherein the targeted regulator binds to the target site and regulates a desired function. The engineered meganuclease DNA-binding domain can be covalently or non-covalently associated with one or more regulatory domains, alternatively two or more regulatory domains, with the two or more domains being two copies of the same domain, or two different domains. The regulatory domains can be covalently linked to the engineered meganuclease DNA-binding domain, e.g., via an amino acid linker, as part of a fusion protein. The engineered meganuclease DNA-binding domains can also be associated with a regulatory domain via a non-covalent dimerization domain, e.g., a leucine zipper, a STAT protein N terminal domain, or an FK506 binding protein (see, e.g., O'Shea, Science. 254:539 (1991), Barahmand-Pour et al., Curr. Top. Microbiol. Immunol. 211:121-128 (1996); Klemm et al., Annu. Rev. Immunol. 16:569-592 (1998); Klemm et al., Annu. Rev. Immunol. 16:569-592 (1998); Ho et al., Nature. 382:822-826 (1996); and Pomeranz et al., Biochem. 37:965 (1998)). The regulatory domain can be associated with the engineered meganuclease DNA-binding domain at any suitable position, including the C- or N-terminus of the engineered meganuclease DNA-binding domain.

Common regulatory domains for addition to the engineered meganuclease DNA-binding domain include, e.g., effector domains from transcription factors (activators, repressors, co-activators, co-repressors), silencers, nuclear hormone receptors, oncogene transcription factors (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g., kinases, acetylases and deacetylases); and DNA modifying enzymes (e.g., methyltransferases, topoisomerases, helicases, ligases, kinases, phosphatases, polymerases, endonucleases) and their associated factors and modifiers.

Transcription factor polypeptides from which one can obtain a regulatory domain include those that are involved in regulated and basal transcription. Such polypeptides include transcription factors, their effector domains, coactivators, silencers, nuclear hormone receptors (see, e.g., Goodrich et al., Cell 84:825-30 (1996) for a review of proteins and nucleic acid elements involved in transcription; transcription factors in general are reviewed in Barnes & Adcock, Clin. Exp. Allergy. 25 Suppl. 2:46-9 (1995) and Roeder, Methods Enzymol. 273:165-71 (1996)). Databases dedicated to transcription factors are known (see, e.g., Science. 269:630 (1995)). Nuclear hormone receptor transcription factors are described in, for example, Rosen et al., J. Med. Chem. 38:4855-74 (1995). The C/EBP family of transcription factors are reviewed in Wedel et al., Immunobiology. 193:171-85 (1995). Coactivators and co-repressors that mediate transcription regulation by nuclear hormone receptors are reviewed in, for example, Meier, Eur. J. Endocrinol. 134 (2): 158-9 (1996); Kaiser et al., Trends Biochem. Sci. 21:342-5 (1996); and Utley et al., Nature. 394:498-502 (1998)). GATA transcription factors, which are involved in regulation of hematopoiesis, are described in, for example, Simon, Nat. Genet. 11:9-11 (1995); Weiss et al., Exp. Hemato. 23:99-107. TATA box binding protein (TBP) and its associated TAF polypeptides (which include TAF30, TAF55, TAF80, TAF110, TAF150, and TAF250) are described in Goodrich & Tjian, Curr. Opin. Cell Biol. 6:403-9 (1994) and Hurley, Curr. Opin. Struct. Biol. 6:69-75 (1996). The STAT family of transcription factors are reviewed in, for example, Barahmand-Pour et al., Curr. Top. Microbiol. Immunol. 211:121-8 (1996). Transcription factors involved in disease are reviewed in Aso et al., J. Clin. Invest. 97:1561-9 (1996).

In one embodiment, the KRAB repression domain from the human KOX-1 protein is used as a transcriptional repressor (Thiesen et al., New Biologist. 2:363-374 (1990); Margolin et al., PNAS. 91:4509-4513 (1994); Pengue et al., Nucl. Acids Res. 22:2908-2914 (1994); Witzgall et al., PNAS. 91:4514-4518 (1994)). In another embodiment, KAP-1, a KRAB co-repressor, is used with KRAB (Friedman et al., Genes Dev. 10:2067-2078 (1996)). Alternatively, KAP-1 can be used alone with a engineered meganuclease DNA-binding domain. Other transcription factors and transcription factor domains that act as transcriptional repressors include MAD (see, e.g., Sommer et al., J. Biol. Chem. 273:6632-6642 (1998); Gupta et al., Oncogene. 16:1149-1159 (1998); Queva et al., Oncogene. 16:967-977 (1998); Larsson et al, Oncogene. 15:737-748 (1997); Laherty et al., Cell. 89:349-356 (1997); and Cultraro et al., Mol. Cell. Biol. 17:2353-2359 (1997); FKHR (forkhead in rhapdosarcoma gene; Ginsberg et al., Cancer Res. 15:3542-3546 (1998); Epstein et al., Mol. Cell. Biol. 18:4118-4130 (1998)); EGR-1 (early growth response gene product-1; Yan et al., PNAS. 95:8298-8303 (1998); and Liu et al., Cancer Gene Ther. 5:3-28 (1998)); the ets2 repressor factor repressor domain (ERD; Sgouras et al., EMBO J. 14:4781-4793 ((1995)); and the MAD smSIN3 interaction domain (SID; Ayer et al., Allol. Cell. Biol. 16:5772-5781 (1996)).

In one embodiment, the HSV VP16 activation domain is used as a transcriptional activator (see, e.g., Hagmann et al., J. Virol. 71:5952-5962 (1997)). Other transcription factors that could supply activation domains include the VP64 activation domain (Seipel et al., EMBO J. 11:4961-4968 (1996)); nuclear hormone receptors (see, e.g., Torchia et al., Curr. Opin. Cell. Biol. 10:373-383 (1998)); the p65 subunit of nuclear factor kappa B (Bitko & Barik, J. Virol. 72:5610-5618 (1998) and Doyle & Hunt, Neuroreport. 8:2937-2942 (1997)); and EGR-1 (early growth response gene product-1; Yan et al., PNAS. 95:8298-8303 (1998); and Liu et al., Cancer Gene Ther. 5:3-28 (1998)).

Kinases, phosphatases, and other proteins that modify polypeptides involved in gene regulation are also useful as regulatory domains for engineered meganuclease DNA-binding domains. Such modifiers are often involved in switching on or off transcription mediated by, for example, hormones.

Kinases involved in transcription regulation are reviewed in Davis, Mol. Reprod. Dev. 42:459-67 (1995), Jackson et al., Adv. Second Messenger Phosphoprotein Res. 28:279-86 (1993), and Boulikas, Crit Rev. Eukaryot. Gene Expr. 5:1-77 (1995), while phosphatases are reviewed in, for example, Schonthal, Semin. Cancer Biol. 6:239-48 (1995). Nuclear tyrosine kinases are described in Wang, Trends Biochem. Sci. 19:373-6 (1994).

As described, useful domains can also be obtained from the gene products of oncogenes (e.g., myc, jun, fos, myb, max, mad, rel, ets, bcl, myb, mos family members) and their associated factors and modifiers. Oncogenes are described in, for example, Cooper, Oncogènes, 2nd ed., The Jones and Bartlett Series in Biology, Boston, MA, Jones and Bartlett Publishers, 1995. The ets transcription factors are reviewed in Waslylk et al., Eur. J. Biochem. 211:7-18 (1993) and Crepieux et al., Crit. Rev. Oncog. 5:615-38 (1994). Myc oncogenes are reviewed in, for example, Ryan et al., Biochem. J. 314:713-21 (1996). The jun and fos transcription factors are described in, for example, The Fos and Jun Families of Transcription Factors, Angel & Herrlich, eds. (1994). The max oncogene is reviewed in Hurlin et al., Cold Spring Harb. Symp. Quant. Biol. 59:109-16. The myb gene family is reviewed in Kanei-Ishii et al., Curr. Top. Microbiol. Immunol. 211:89-98 (1996). The mos family is reviewed in Yew et al., Curr. Opin. Genet. Dev. 3:19-25 (1993).

Engineered meganuclease DNA-binding domains can include regulatory domains obtained from DNA repair enzymes and their associated factors and modifiers. DNA repair systems are reviewed in, for example, Vos, Curr. Opin. Cell Biol. 4:385-95 (1992); Sancar, Ann. Rev. Genet. 29:69-105 (1995); Lehmann, Genet. Eng. 17:1-19 (1995); and Wood, Ann. Rev. Biochem. 65:135-67 (1996).

DNA rearrangement enzymes and their associated factors and modifiers can also be used as regulatory domains (see, e.g., Gangloff et al., Experientia. 50:261-9 (1994); Sadowski, FASEB J. 7:760-7 (1993)).

Similarly, regulatory domains can be derived from DNA modifying enzymes (e.g., DNA methyltransferases, topoisomerases, helicases, ligases, kinases, phosphatases, polymerases) and their associated factors and modifiers. Helicases are reviewed in Matson et al., Bioessays, 16:13-22 (1994), and methyltransferases are described in Cheng, Curr. Opin. Struct. Biol. 5:4-10 (1995). Chromatin associated proteins and their modifiers (e.g., kinases, acetylases and deacetylases), such as histone deacetylase (Wolffe, Science. 272:371-2 (1996)) are also useful as domains for addition to the engineered meganuclease DNA-binding domain of choice. In one embodiment, the regulatory domain is a DNA methyl transferase that acts as a transcriptional repressor (see, e.g., Van den Wyngaert et al., FEBS Lett. 426:283-289 (1998); Flynn et al., J. Mol. Biol. 279:101-116 (1998); Okano et al., Nucleic Acids Res. 26:2536-2540 (1998); and Zardo & Caiafa, J. Biol. Chem. 273:16517-16520 (1998)).

Factors that control chromatin and DNA structure, movement and localization and their associated factors and modifiers; factors derived from microbes (e.g., prokaryotes, eukaryotes and virus) and factors that associate with or modify them can also be used to obtain chimeric proteins. In one embodiment, recombinases and integrases are used as regulatory domains. In one embodiment, histone acetyltransferase is used as a transcriptional activator (see, e.g., Jin & Scotto, Mol. Cell. Biol. 18:4377-4384 (1998); Wolffe, Science. 272:371-372 (1996); Taunton et al., Science. 272:408-411 (1996); and Hassig et al., PNAS. 95:3519-3524 (1998)). In another embodiment, histone deacetylase is used as a transcriptional repressor (see, e.g., Jin & Scotto, Mol. Cell. Biol. 18:4377-4384 (1998); Syntichaki & Thireos, J. Biol. Chem. 273:24414-24419 (1998); Sakaguchi et al., Genes Dev. 12:2831-2841 (1998); and Martinez et al., J. Biol. Chem. 273:23781-23785 (1998)).

Another suitable repression domain is methyl binding domain protein 2B (MBD-2B) (see, also Hendrich et al. (1999) Mamm Genome. 10:906-912 for description of MBD proteins). Another useful repression domain is that associated with the v-ErbA protein (see infra). See, for example, Damm, et al. (1989) Nature. 339:593-597; Evans (1989) Int. J. Cancer Suppl. 4:26-28; Pain et al. (1990) New Biol. 2:284-294; Sap et al. (1989) Nature. 340:242-244; Zenke et al. (1988) Cell. 52:107-119; and Zenke et al. (1990) Cell. 61:1035-1049. Additional exemplary repression domains include, but are not limited to, thyroid hormone receptor (TR, see inf7a), SID, MBD1, MBD2, MBD3, MBD4, MBD-like proteins, members of the DNMT family (e.g., DNMT1, DNMT3A, DNMT3B), Rb, MeCP1 and MeCP2. See, for example, Bird et al. (1999) Cell. 99:451-454; Tyler et al. (1999) Cell. 99:443-446; Knoepfler et al. (1999) Cell. 99:447-450; and Robertson et al. (2000) Nature Genet. 25:338-342. Additional exemplary repression domains include, but are not limited to, ROM2 and AtHD2A. See, for example, Chern et al. (1996) Plant Cell. 8:305-321; and Wu et al. (2000) Plant J. 22:19-27.

Certain members of the nuclear hormone receptor (NHR) superfamily, including, for example, thyroid hormone receptors (TRs) and retinoic acid receptors (RARs) are among the most potent transcriptional regulators currently known. Zhang et al., Annu. Rev. Physio. 62:439-466 (2000) and Sucov et al., Mol Neurobiol. 10 (2-3): 169-184 (1995). In the absence of their cognate ligand, these proteins bind with high specificity and affinity to short stretches of DNA (e.g., 12-17 base pairs) within regulatory loci (e.g., enhancers and promoters) and effect robust transcriptional repression of adjacent genes.

The potency of their regulatory action stems from the concurrent use of two distinct functional pathways to drive gene silencing: (i) the creation of a localized domain of repressive chromatin via the targeting of a complex between the corepressor N-CoR and a histone deacetylase, HDAC3 (Guenther et al., Genes Dev. 14:1048-1057 (2000); Umov et al., EMBO J. 19:4074-4090 (2000); Li et al., EMBO J. 19, 4342-4350 (2000) and Underhill et al., J. Biol. Chem. 275:40463-40470 (2000)) and (ii) a chromatin independent pathway (Urnov et al., supra) that may involve direct interference with the function of the basal transcription machinery (Fondell et al., Genes Dev. 7 (7B): 1400-1410 (1993) and Fondell et al., Mol Cell Biol. 16:281-287 (1996).

In the presence of very low (e.g., nanomolar) concentrations of their ligand, these receptors undergo a conformational change which leads to the release of corepressors, recruitment of a different class of auxiliary molecules (e.g., coactivators) and potent transcriptional activation. Collingwood et al., J. Mol. Endocrinol. 23 (3): 255-275 (1999).

The portion of the receptor protein responsible for transcriptional control (e.g., repression and activation) can be physically separated from the portion responsible for DNA binding, and retains full functionality when tethered to other polypeptides, for example, other DNA-binding domains. Accordingly, a nuclear hormone receptor transcription control domain can be fused to a engineered meganuclease DNA-binding domain such that the transcriptional regulatory activity of the receptor can be targeted to a chromosomal region of interest (e.g., a gene) by virtue of the engineered meganuclease DNA-binding domain.

Moreover, the structure of TR and other nuclear hormone receptors can be altered, either naturally or through recombinant techniques, such that it loses all capacity to respond to hormone (thus losing its ability to drive transcriptional activation), but retains the ability to effect transcriptional repression. This approach is exemplified by the transcriptional regulatory properties of the oncoprotein v-ErbA. The v-ErbA protein is one of the two proteins required for leukemic transformation of immature red blood cell precursors in young chicks by the avian erythroblastosis virus. TR is a major regulator of erythropoiesis (Beug et al., Biochim Biophys Acta. 1288 (3): M35-47 (1996); in particular, in its unliganded state, it represses genes required for cell cycle arrest and the differentiated state. Thus, the administration of thyroid hormone to immature erythroblasts leads to their rapid differentiation. The v-ErbA oncoprotein is an extensively mutated version of TR; these mutations include: (i) deletion of 12 amino-terminal amino acids; (ii) fusion to the gag oncoprotein; (iii) several point mutations in the DNA binding domain that alter the DNA binding specificity of the protein relative to its parent, TR, and impair its ability to heterodimerize with the retinoid X receptor; (iv) multiple point mutations in the ligand-binding domain of the protein that effectively eliminate the capacity to bind thyroid hormone; and (v) a deletion of a carboxy-terminal stretch of amino acids that is essential for transcriptional activation. Stunnenberg et al., Biochim Biophys Acta. 1423 (1): F15-33 (1999). As a consequence of these mutations, v-ErbA retains the capacity to bind to naturally occurring TR target genes and is an effective transcriptional repressor when bound (Umov et al., supra; Sap et al., Nature. 340:242-244 (1989); and Ciana et al., EMBO J. 17 (24): 7382-7394 (1999). In contrast to TR, however, v-ErbA is completely insensitive to thyroid hormone, and thus maintains transcriptional repression in the face of a challenge from any concentration of thyroids or retinoids, whether endogenous to the medium, or added by the investigator.

This functional property of v-ErbA is retained when its repression domain is fused to a heterologous, synthetic DNA binding domain. Accordingly, in one aspect, v-ErbA or its functional fragments are used as a repression domain. In additional embodiments, TR or its functional domains are used as a repression domain in the absence of ligand and/or as an activation domain in the presence of ligand (e.g., 3,5, 3′-triiodo-L-thyronine or T3).

Thus, TR can be used as a switchable functional domain (i.e., a bifunctional domain); its activity (activation or repression) being dependent upon the presence or absence (respectively) of ligand.

Additional exemplary repression domains are obtained from the DAX protein and its functional fragments. Zazopoulos et al., Nature. 390:311-315 (1997). In particular, the C-terminal portion of DAX-1, including amino acids 245-470, has been shown to possess repression activity. Altincicek et al., J. Biol. Ther. 275:7662-7667 (2000). A further exemplary repression domain is the RBP1 protein and its functional fragments. Lai et al., Oncogene 18:2091-2100 (1999); Lai et al., Mol. Cell. Biol. 19:6632-6641 (1999); Lai et al., Mol. Cell. Biol. 21:2918-2932 (2001) and WO 01/04296. The full-length RBP1 polypeptide contains 1257 amino acids. Exemplary functional fragments of RBP1 are a polypeptide comprising amino acids 1114-1257, and a polypeptide comprising amino acids 243-452.

Members of the TIEG family of transcription factors contain three repression domains known as R1, R2 and R3. Repression by TIEG family proteins is achieved at least in part through recruitment of mSIN3A histone deacetylases complexes. Cook et al. (1999) J. Biol. Chem. 274:29, 500-29, 504; Zhang et al. (2001) Mol. Cell. Biol. 21:5041-5049. Any or all of these repression domains (or their functional fragments) can be fused alone, or in combination with additional repression domains (or their functional fragments), to a DNA-binding domain to generate a targeted exogenous repressor molecule.

Furthermore, the product of the human cytomegalovirus (HCMV) UL34 open reading frame acts as a transcriptional repressor of certain HCMV genes, for example, the US3 gene. LaPierre et al. (2001) J. Virol. 75:6062-6069. Accordingly, the UL34 gene product, or functional fragments thereof, can be used as a component of a fusion polypeptide also comprising a zinc finger binding domain. Nucleic acids encoding such fusions are also useful in the methods and compositions disclosed herein.

Yet another exemplary repression domain is the CDF-1 transcription factor and/or its functional fragments. See, for example, WO 99/27092.

The Ikaros family of proteins are involved in the regulation of lymphocyte development, at least in part by transcriptional repression. Accordingly, an Ikaros family member (e.g., Ikaros, Aiolos) or a functional fragment thereof, can be used as a repression domain. See, for example, Sabbattini et al. (2001) EMBO J. 20:2812-2822.

The yeast Ashlp protein comprises a transcriptional repression domain. Maxon et al. (2001) Proc. Natl. Acad. Sci. USA 98:1495-1500. Accordingly, the Ashlp protein, its functional fragments, and homologues of Ashlp, such as those found, for example, in, vertebrate, mammalian, and plant cells, can serve as a repression domain for use in the methods and compositions disclosed herein.

Additional exemplary repression domains include those derived from histone deacetylases (HDACs, e.g., Class I HDACs, Class II HDACs, SIR-2 homologues), HDAC-interacting proteins (e.g., SIN3, SAP30, SAP15, NCOR, SMRT, RB, p107, p130, RBAP46/48, MTA, Mi-2, Brgl, Brm), DNA-cytosine methyltransferases (e.g., Dnmt1, Dnmt3a, Dnmt3b), proteins that bind methylated DNA (e.g., MBD1, MBD2, MBD3, MBD4, MeCP2, DMAP1), protein methyltransferases (e.g., lysine and arginine methylases, SuVar homologues such as Suv39Hl), polycomb-type repressors (e.g., Bmi-1, eed1, RING1, RYBP, E2F6, Mell8, YY1 and CtBP), viral repressors (e.g., adenovirus Elb 55K protein, cytomegalovirus UL34 protein, viral oncogenes such as v-erbA), hormone receptors (e.g., Dax-1, estrogen receptor, thyroid hormone receptor), and repression domains associated with naturally-occurring zinc finger proteins (e.g., WT1, KAP1). Further exemplary repression domains include members of the polycomb complex and their homologues, HPH1, HPH2, HPC2, NC2, groucho, Eve, tramtrak, mHP1, SIP1, ZEB1, ZEB2, and Enx1/Ezh2. In all of these cases, either the full-length protein or a functional fragment can be used as a repression domain for fusion to a zinc finger binding domain. Furthermore, any homologues of the aforementioned proteins can also be used as repression domains, as can proteins (or their functional fragments) that interact with any of the aforementioned proteins.

Additional repression domains, and exemplary functional fragments, are as follows. Hes1 is a human homologue of the Drosophila hairy gene product and comprises a functional fragment encompassing amino acids 910-1014. In particular, a WRPW (trp-arg-pro-trp) motif can act as a repression domain. Fisher et al (1996) Mol. Cell. Biol. 16:2670-2677.

The TLE1, TLE2 and TLE3 proteins are human homologues of the Drosophila groucho gene product. Functional fragments of these proteins possessing repression activity reside between amino acids 1-400. Fisher et al., supra.

The Tbx3 protein possesses a functional repression domain between amino acids 524-721. He et al. (1999) Proc. Natl. Acad. Sci. USA 96:10, 212-10, 217. The Tbx2 gene product is involved in repression of the p14/p16 genes and contains a region between amino acids 504-702 that is homologous to the repression domain of Tbx3; accordingly Tbx2 and/or this functional fragment can be used as a repression domain. Carreira et al. (1998) Mol. Cell. Biol. 18:5, 099-5, 108.

The human Ezh2 protein is a homologue of Drosophila e7lha7lcer of zeste and recruits the eed1 polycomb-type repressor. A region of the Ezh2 protein comprising amino acids 1-193 can interact with eed1 and repress transcription; accordingly Ezh2 and/or this functional fragment can be used as a repression domain. Denisenko et al. (1998) Mol. Cell. Biol. 18:5634-5642.

The RYBP protein is a corepressor that interacts with polycomb complex members and with the YY1 transcription factor. A region of RYBP comprising amino acids 42-208 has been identified as functional repression domain. Garcia et al. (1999) EMBO J. 18:3404-3418.

The RING finger protein RING 1 A is a member of two different vertebrate polycomb-type complexes, contains multiple binding sites for various components of the polycomb complex, and possesses transcriptional repression activity. Accordingly, RING 1 A or its functional fragments can serve as a repression domain. Satjin et al. (1997) Mol. Cell. Biol. 17:4105-4113.

The Bmi-1 protein is a member of a vertebratepolycomb complex and is involved in transcriptional silencing. It contains multiple binding sites for various polycomb complex components. Accordingly, Bmi-1 and its functional fragments are useful as repression domains. Gunster et al. (1997) Mol. Cell. Biol. 17:2326-2335; Hemenway et al. (1998) Oncogen. 16:2541-2547.

The E2F6 protein is a member of the mammalian Bmi-1-containing polycomb complex and is a transcriptional repressor that is capable or recruiting RYBP, Bmi-1 and RING1A. A functional fragment of E2F6 comprising amino acids 129-281 acts as a transcriptional repression domain. Accordingly, E2F6 and its functional fragments can be used as repression domains. Trimarchi et al. (2001) Proc Natl. Acad. Sci. USA 98:1519-1524.

The eed1 protein represses transcription at least in part through recruitment of histone deacetylases (e.g., HDAC2). Repression activity resides in both the N- and C-terminal regions of the protein. Accordingly, eed1 and its functional fragments can be used as repression domains. van der Vlag et al. (1999) Nature Genet. 23:474-478.

The CTBP2 protein represses transcription at least in part through recruitment of an HPC2-polycomb complex. Accordingly, CTBP2 and its functional fragments are useful as repression domains. Richard et al. (1999) Mol. Cell. Biol. 19:777-787.

Neuron-restrictive silencer factors are proteins that repress expression of neuron-specific genes. Accordingly, a NRSF or functional fragment thereof can serve as a repression domain. See, for example, U.S. Pat. No. 6,270,990.

It will be clear to those of skill in the art that any repressor or a molecule that interacts with a repressor is suitable as a functional domain. Essentially any molecule capable of recruiting a repressive complex and/or repressive activity (such as, for example, histone deacetylation) to the target gene is useful as a repression domain of a fusion protein.

Additional exemplary activation domains include, but are not limited to, p300, CBP, PCAF, SRC1 PvALF, AtHD2A and ERF-2. See, for example, Robyr et al. (2000) Mol. Endocrinol. 14:329-347; Collingwood et al. (1999) J. Mol. Endocrinol. 23:255-275; Leo et al. (2000) Gene 245:1-11; Manteuffel-Cymborowska (1999) Acta Biochim. Pol. 46:77-89; McKenna et al. (1999) J. Steroid Biochem. Mol. Biol. 69:3-12; Malik et al. (2000) Trends Biochem. Sci. 25:277-283; and Lemon et al. (1999) Curr. Opin. Genet. Dev. 9:499-504. Additional exemplary activation domains include, but are not limited to, OsGAI, HALF-1, C1, AP1, ARF-5, -6, -7, and -8, CPRF1, CPRF4, MYC-RP/GP, and TRAB1. See, for example, Ogawa et al. (2000) Gene. 245:21-29; Okanami et al. (1996) Genes Cells. 1:87-99; Goff et al. (1991) Genes Dev. 5:298-309; Cho et al. (1999) Plant Mol. Biol. 40:419-429; Ulmason et al. (1999) Proc. Natl. Acad. Sci. USA 96:5844-5849; Sprenger-Haussels et al. (2000) Plant J. 22:1-8; Gong et al. (1999) Plant Mol. Biol. 41:33-44; and Hobo et al. (1999) Proc. Natl. Acad. Sci. USA 96:15348-15353.

It will be clear to those of skill in the art that any activator or a molecule that interacts with an activator is suitable as a functional domain. Essentially any molecule capable of recruiting an activating complex and/or activating activity (such as, for example, histone acetylation) to the target gene is useful as an activating domain of a fusion protein.

Insulator domains, chromatin remodeling proteins such as ISWI-containing domains and/or methyl binding domain proteins suitable for use as functional domains in fusion molecules are described, for example, in co-owned WO 01/83793; WO 02/26959; WO 02/26960 and WO 02/44376.

In a further embodiment, an engineered meganuclease DNA-binding domain is fused to a bifunctional domain (BFD). A bifunctional domain is a transcriptional regulatory domain whose activity depends upon interaction of the BFD with a second molecule. The second molecule can be any type of molecule capable of influencing the functional properties of the BFD including, but not limited to, a compound, a small molecule, a peptide, a protein, a polysaccharide or a nucleic acid. An exemplary BFD is the ligand binding domain of the estrogen receptor (ER). In the presence of estradiol, the ER ligand binding domain acts as a transcriptional activator; while, in the absence of estradiol and the presence of tamoxifen or 4-hydroxy-tamoxifen, it acts as a transcriptional repressor. Another example of a BFD is the thyroid hormone receptor (TR) ligand binding domain which, in the absence of ligand, acts as a transcriptional repressor and in the presence of thyroid hormone (T3), acts as a transcriptional activator.

An additional BFD is the glucocorticoid receptor (GR) ligand binding domain. In the presence of dexamethasone, this domain acts as a transcriptional activator; while, in the presence of RU486, it acts as a transcriptional repressor. An additional exemplary BFD is the ligand binding domain of the retinoic acid receptor. In the presence of its ligand all-trans-retinoic acid, the retinoic acid receptor recruits a number of co-activator complexes and activates transcription. In the absence of ligand, the retinoic acid receptor is not capable of recruiting transcriptional co-activators. Additional BFDs are known to those of skill in the art. See, for example, U.S. Pat. Nos. 5,834,266 and 5,994,313 and WO 99/10508.

Another class of functional domains, derived from nuclear receptors, are those whose functional activity is regulated by a non-natural ligand. These are often mutants or modified versions of naturally-occurring receptors and are sometimes referred to as “switchable” domains. For example, certain mutants of the progesterone receptor (PR) are unable to interact with their natural ligand, and are therefore incapable of being transcriptionally activated by progesterone. Certain of these mutants, however, can be activated by binding small molecules other than progesterone (one example of which is the antiprogestin mifepristone). Such non-natural but functionally competent ligands have been denoted anti-hormones. See, e.g., U.S. Pat. Nos. 5,364,791; 5,874,534; 5,935,934; Wang et al., (1994) Proc. Natl. Acad. Sci. USA 91:8180-8184; Wang et al., (1997) Gene Ther. 4:432-441.

Accordingly, a fusion comprising a targeted engineered meganuclease DNA-binding domain, a functional domain, and a mutant PR ligand binding domain of this type can be used for mifepristone-dependent activation or repression of an endogenous gene of choice, by designing the engineered meganuclease DNA-binding domain such that it binds in or near the gene of choice. If the fusion contains an activation domain, mifepristone-dependent activation of gene expression is obtained; if the fusion contains a repression domain, mifepristone-dependent repression of gene expression is obtained. Additionally, polynucleotides encoding such fusion proteins are provided, as are vectors comprising such polynucleotides and cells comprising such polynucleotides and vectors. It will be clear to those of skill in the art that modified or mutant versions of receptors other than PR can also be used as switchable domains. See, for example, Tora et al. (1989) EMBO J. 8:1981-1986.

### 11. Expression Vectors

The nucleic acid encoding the targeted transcriptional effector of choice is typically cloned into intermediate vectors for transformation into prokaryotic or eukaryotic cells for replication and/or expression, e.g., for determination of K_{d}. Intermediate vectors are typically prokaryote vectors, e.g., plasmids, or shuttle vectors, or insect vectors, for storage or manipulation of the nucleic acid encoding engineered meganuclease DNA-binding domain or production of protein. The nucleic acid encoding a engineered meganuclease DNA-binding domain is also typically cloned into an expression vector, for administration to a plant cell, animal cell (e.g., a human or other mammalian cell), fungal cell, bacterial cell, or protozoal cell.

To obtain expression of a cloned gene or nucleic acid, a engineered meganuclease DNA-binding domain is typically subcloned into an expression vector that contains a promoter to direct transcription.

Suitable bacterial and eukaryotic promoters are well known in the art and described, e.g., in Sambrook et al., Molecular Cloning, A Laboratory Manual (2nd ed. 1989); Kriegler, Gene Trtisfei- and Expression: A Laboratory Manual (1990); and Current Protocols in Molecular Biology (Ausubel et al., eds., 1994). Bacterial expression systems for expressing the ZFP are available in, e.g., E. coli, Bacillus sp., and Salmonella (Palva et al., Gene. 22:229-235 (1983)). Kits for such expression systems are commercially available. Eukaryotic expression systems for mammalian cells, yeast, and insect cells are well known in the art and are also commercially available.

The promoter used to direct expression of a targeted transcriptional effector nucleic acid depends on the particular application. For example, a strong constitutive promoter can be used for expression and purification of targeted transcriptional effector. In contrast, when a targeted transcriptional effector is administered in vivo for gene regulation, either a constitutive or an inducible promoter can be used, depending on the particular use of the targeted transcriptional effector. In addition, a promoter for administration of a targeted transcriptional effector can be a weak promoter, such as HSV TK, or a promoter having similar activity. The promoter also can include elements that are responsive to transactivation, e.g., hypoxia response elements, Gal4 response elements, lac repressor response element, and small molecule control systems such as tet-regulated systems and the RU-486 system (see, e.g., Gossen & Bujard, PNAS. 89:5547 (1992); Oligino et al., Gene Ther. 5:491-496 (1998); Wang et al., Gene Ther. 4:432-441 (1997); Neering et al., Blood. 88:1147-1155 (1996); and Rendahl et al., Nat. Biotechnol. 16:757-761 (1998)).

In addition to the promoter, the expression vector can contain a transcription unit or expression cassette that contains all the additional elements required for the expression of the nucleic acid in host cells, either prokaryotic or eukaryotic. An expression cassette can contain a promoter operably linked, e.g., to the nucleic acid sequence encoding the targeted transcriptional effector, and signals required, e.g., for efficient polyadenylation of the transcript, transcriptional termination, ribosome binding sites, or translation termination.

Additional elements of the cassette may include, e.g., enhancers, and heterologous spliced intronic signals.

The particular expression vector used to transport the genetic information into the cell is selected with regard to the intended use of the targeted transcriptional effector, e.g., expression in plants, animals, bacteria, fungus, protozoa etc. Standard bacterial expression vectors include plasmids such as pBR322 based plasmids, pSKF, pET23D, and commercially available fusion expression systems such as GST and LacZ. A common fusion protein is the maltose binding protein, “MBP.” Such fusion proteins are used for purification of the targeted transcriptional effector. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMT010/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with a targeted transcriptional effector encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, naked DNA, plasmid vectors, viral vectors, both episomal and integrative, and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

### 12. Assays for Determining Regulation of Gene Expression

A variety of assays can be used to determine the level of gene expression regulation by targeted transcriptional effectors. The activity of a particular targeted transcriptional effector can be assessed using a variety of ill vitro and in vivo assays, by measuring, e.g., protein or mRNA levels, product levels, enzyme activity, tumor growth; transcriptional activation or repression of a reporter gene; second messenger levels (e.g., cGMP, CAMP, IP3, DAG, Ca2+); cytokine and hormone production levels; and neovascularization, using, e.g., immunoassays (e.g., ELISA and immunohistochemical assays with antibodies), hybridization assays (e.g., RNase protection, northerns, in situ hybridization, oligonucleotide array studies), colorimetric assays, amplification assays, enzyme activity assays, tumor growth assays, phenotypic assays, and the like.

Targeted transcriptional effectors can be tested for activity in vitro using cultured cells, e.g., HEK 293 cells, CHO cells, VERO cells, BHK cells, HeLa cells, COS cells, and the like. The targeted transcriptional effectors is often first tested using a transient expression system with a reporter gene, and then regulation of the target endogenous gene is tested in cells and in animals, both in vivo and ex vivo. The targeted transcriptional effector can be recombinantly expressed in a cell, recombinantly expressed in cells transplanted into an animal, or recombinantly expressed in a transgenic animal, as well as administered as a protein to an animal or cell using delivery vehicles described below. The cells can be immobilized, be in solution, be injected into an animal, or be naturally occurring in a transgenic or non-transgenic animal.

Modulation of gene expression is tested using one of the in vitro or in vivo assays described herein. Samples or assays are treated with a targeted transcriptional effector and compared to control samples without the test compound, to examine the extent of modulation. As described above, for regulation of endogenous gene expression, the targeted transcriptional effector typically has a K_{d} of 200 nM or less, or 100 nM or less, or 50 nM or less, or 25 nM or less.

The effects of the targeted transcriptional effectors can be measured by examining any of the parameters described above. Any suitable gene expression, phenotypic, or physiological change can be used to assess the influence of a targeted transcriptional effector. When the functional consequences are determined using intact cells or animals, one can also measure a variety of effects such as tumor growth, neovascularization, hormone release, transcriptional changes to both known and uncharacterized genetic markers (e.g., northern blots or oligonucleotide array studies), changes in cell metabolism such as cell growth or pH changes, and changes in intracellular second messengers such as cGMP.

Assays for targeted transcriptional effector regulation of endogenous gene expression can be performed in vitro. In one useful in vitro assay format, targeted transcriptional effector regulation of endogenous gene expression in cultured cells is measured by examining protein production using an ELISA assay. The test sample is compared to control cells treated with an empty vector or an unrelated targeted transcriptional effector that is targeted to another gene.

In another embodiment, targeted transcriptional effector regulation of endogenous gene expression is determined in vitro by measuring the level of target gene mRNA expression. The level of gene expression is measured using amplification, e.g., using PCR, LCR, or hybridization assays, e.g., northern hybridization, RNase protection, dot blotting. RNase protection is used in one embodiment. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system can be devised using the target gene promoter operably linked to a reporter gene such as luciferase, green fluorescent protein, CAT, or p-gal. The reporter construct is typically co-transfected into a cultured cell.

After treatment with the targeted transcriptional effector of choice, the amount of reporter gene transcription, translation, or activity is measured according to standard techniques known to those of skill in the art.

Another example of an assay format useful for monitoring targeted transcriptional effector regulation of endogenous gene expression is performed in vivo. This assay is particularly useful for examining targeted transcriptional effectors that inhibit expression of tumor promoting genes, genes involved in tumor support, such as neovascularization (e.g., VEGF), or that activate tumor suppressor genes such as p53. In this assay, cultured tumor cells expressing the targeted transcriptional effector of choice are injected subcutaneously into an immune compromised mouse such as an athymic mouse, an irradiated mouse, or a SCID mouse. After a suitable length of time (e.g., 4-8 weeks), tumor growth is measured, e.g., by volume or by its two largest dimensions, and compared to the control. Tumors that have statistically significant reduction (using, e.g., Student's T test) are said to have inhibited growth. Alternatively, the extent of tumor neovascularization can also be measured. Immunoassays using endothelial cell specific antibodies are used to stain for vascularization of the tumor and the number of vessels in the tumor. Tumors that have a statistically significant reduction in the number of vessels (using, e.g., Student's T test) are said to have inhibited neovascularization.

Transgenic and non-transgenic animals are also used in some embodiments for examining regulation of endogenous gene expression in vivo. Transgenic animals typically express the targeted transcriptional effector of choice. Alternatively, animals that transiently express the ZFP of choice, or to which the targeted transcriptional effector has been administered in a delivery vehicle, can be used. Regulation of endogenous gene expression is tested using any one of the assays described herein.

### 13. Nucleic Acids Encoding Fusion Proteins

Conventional viral and non-viral based gene transfer methods can be used to introduce nucleic acids encoding targeted transcriptional effector in mammalian cells or target tissues. Such methods can be used to administer nucleic acids encoding targeted transcriptional effectors to cells in vitro.

The nucleic acids encoding targeted transcriptional effectors can be administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science. 256:808-813 (1992); Nabel & Felgner, TIBTECH. 11:211-217 (1993); Mitani & Caskey, TIBTECH. 11:162-166 (1993); Dillon, TIBTECH. 11:167-175 (1993); Miller, Nature. 357:455-460 (1992); Van Brunt, Biotechnology. 6 (10): 1149-1154 (1988); Vigne, Restorative Neurology and Neuroscience. 8:35-36 (1995); Kremer & Perricaudet, British Medical Bulletin. 51 (1): 31-44 (1995); Haddada et al., in Current Topics in Microbiology and Immunology. Doerfler and Böhm (eds) (1995); and Yu et al., Gene Therapy. 1:13-26 (1994).

Methods of non-viral delivery of nucleic acids encoding targeted transcriptional effectors include lipofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin). Cationic and neutral lipids that are suitable for efficient receptor-recognition lipofection of polynucleotides include those of Felgner, WO 91/17424 and WO 91/16024. Delivery can be to cells (ex vivo administration) or target tissues (in vivo administration).

The preparation of lipid: nucleic acid complexes, including targeted liposomes such as immunolipid complexes, is well known to one of skill in the art (see, e.g., Crystal, Science. 270: 404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy. 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids encoding a targeted transcriptional effector take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of targeted transcriptional effectors could include retroviral, lentivirus, adenoviral, adeno-associated and herpes simplex virus vectors for gene transfer. Viral vectors are currently the most efficient and versatile method of gene transfer in target cells and tissues.

Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vector that are able, to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscheretal., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression of the targeted transcriptional effector is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology. 160: 38-47 (1987); U.S. Pat. No. 4,797,368; WO 93/24641; Kotin, Human Gene Therapy. 5: 793-801 (1994); Muzyczka, J. Clin. Invest. 94: 1351 (1994). Construction of recombinant AAV vectors are described in a number of publications, including U.S. Pat. No. 5,173,414; Tratschin et al., Mol. Cell. Biol. 5: 3251-3260 (1985); Tratschin, et al., Mol. Cell. Biol. 4:2072-2081 (1984); Hermonat & Muzyczka, PNAS. 81:6466-6470 (1984); and Samulski et al., J. Virol. 63:03822-3828 (1989).

In particular, at least six viral vector approaches are currently available for gene transfer in clinical trials, with retroviral vectors by far the most frequently used system.

All of these viral vectors utilize approaches that involve complementation of defective vectors by genes inserted into helper cell lines to generate the transducing agent. pLASN and MFG-S are examples are retroviral vectors that have been used in clinical trials (Dunbar et al., Blood. 85:3048-305 (1995); Kohn et al., Nat. Med. 1:1017-102 (1995); Malech et al., PNAS. 94:22 12133-12138 (1997)). PA317/pLASN was the first therapeutic vector used in a gene therapy trial. (Blaese et al., Science. 270:475-480 (1995)). Transduction efficiencies of 50% or greater have been observed for MFG-S packaged vectors. (Ellem et al., Cancer Immunol. Immunother. 44 (1): 10-20 (1997); Dranoff et al., Hum. Gene Ther. 1:111-2 (1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet. 351:9117 1702-3 (1998), Kearns et al., Gene Ther. 9:748-55 (1996)).

Replication-deficient recombinant adenoviral vectors (Ad) are predominantly used for colon cancer gene therapy, because they can be produced at high titer and they readily infect a number of different cell types. Most adenovirus vectors are engineered such that a transgene replaces the Ad Ela, Elb, and E3 genes; subsequently the replication defector vector is propagated in human 293 cells that supply deleted gene function in trans. Ad vectors can transduce multiply types of tissues in vivo, including nondividing, differentiated cells such as those found in the liver, kidney and muscle system tissues.

Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection. 24:1 5-10 (1996); Sterman et al, Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include HEK 293 cells, which package adenovirus, and W2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., PNAS 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In one embodiment, cells are isolated from the subject organism, transfected with a targeted transcriptional effector nucleic acid (gene or cDNA), and re-infused back into the subject organism (such as a patient). Various cell types suitable for ex vivo transfection are well known to those of skill in the art (see, e.g., Freshney et al., Culture of Animal Cells, A Manual of Basic Sechnique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-y and TNF-a are known (see Inaba et al, J. Exp. Med. 176:1693-1702 (1992)).

Stem cells are isolated for transduction and differentiation using known methods.

For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (see Inaba et al., J. Exp. Med. 176:1693-1702 (1992)).

Vectors (e.g., retroviruses, adenoviruses, liposomes, etc.) containing therapeutic targeted transcriptional effector nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

### 14. Delivery Vehicles

An important factor in the administration of polypeptide compounds, such as the targeted transcriptional effectors, is ensuring that the polypeptide has the ability to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins and other compounds such as liposomes have been described, which have the ability to translocate polypeptides such as targeted transcriptional effectors across a cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest intemalizable peptide of a homeodomain protein, Antennapedia, was found to be the third helix of the protein, from amino acid position 43 to 58 (see, e.g., Prochiantz, Current Opinion in Neurobiology 6:629-634 (1996)). Another subsequence, the h (hydrophobic) domain of signal peptides, was found to have similar cell membrane translocation characteristics (see, e.g., Lin et al., J. Biol. Chem. 270:1 4255-14258 (1995)).

Examples of peptide sequences which can be linked to a protein, for facilitating uptake of the protein into cells, include, but are not limited to: an 11 amino acid peptide of the tat protein of HIV; a 20 residue peptide sequence which corresponds to amino acids 84-103 of the pl6 protein (see Fahraeus et al., Current Biology. 6:84 (1996)); the third helix of the 60-amino acid long homeodomain of Antennapedia (Derossi et al., J. Biol. Chem. 269:10444 (1994)); the h region of a signal peptide such as the Kaposi fibroblast growth factor (K-FGF) h region (Lin et al., supra); or the VP22 translocation domain from HSV (Elliot & O'Hare, Cell. 88:223-233 (1997)). Other suitable chemical moieties that provide enhanced cellular uptake may also be chemically linked to targeted transcriptional effectors.

Toxin molecules also have the ability to transport polypeptides across cell membranes. Often, such molecules are composed of at least two parts (called “binary toxins”): a translocation or binding domain or polypeptide and a separate toxin domain or polypeptide. Typically, the translocation domain or polypeptide binds to a cellular receptor, and then the toxin is transported into the cell. Several bacterial toxins, including Clostridium perfrisagens iota toxin, diphtheria toxin (DT), Pseudomonas exotoxin A (PE), pertussis toxin (PT), Bacillus aitthracis toxin, and pertussis adenylate cyclase (CYA), have been used in attempts to deliver peptides to the cell cytosol as internal or amino-terminal fusions (Arora et al., J. Biol. Chem., 268:3334-3341 (1993); Perelle et al., Infect. Immun., 61:5147-5156 (1993); Stenmark et al., J. Cell Biol. 113:1025-1032 (1991); Donnelly et al., PNAS. 90:3530-3534 (1993); Carbonetti et al., Abstr. Annu. Meet. Am. Soc. Microbiol. 95:295 (1995); Sebo et al., Infect. Immun. 63:3851-3857 (1995); Klimpel et al., PNAS. 89:10277-10281 (1992); and Novak et al., J. Biol. Chem. 267:17186-17193 1992)).

Amino acid sequences which facilitate internalization of linked polypeptides into cells can be selected from libraries of randomized peptide sequences. See, for example, Yeh et al. (2003) Molecular Therapy. 7 (5): S461 (Abstract #1191). Such “internalization peptides” can be fused to a targeted transcriptional effector to facilitate entry of the protein into a cell.

Such subsequences, as described above, can be used to translocate targeted transcriptional effectors across a cell membrane. ZFPs can be conveniently fused to or derivatized with such sequences.

Typically, the translocation sequence is provided as part of a fusion protein. Optionally, a linker can be used to link the targeted transcriptional effector and the translocation sequence. Any suitable linker can be used, e.g., a peptide linker.

The targeted transcriptional effector can also be introduced into an animal cell (e.g., a mammalian cell) via a liposomes and liposome derivatives such as immunoliposomes. The term “liposome” refers to vesicles comprised of one or more concentrically ordered lipid bilayers, which encapsulate an aqueous phase. The aqueous phase typically contains the compound to be delivered to the cell, i.e., a targeted transcriptional effector.

The liposome fuses with the plasma membrane, thereby releasing the drug into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound (in this case, a targeted transcriptional effector) at the target tissue or cell. For systemic or tissue specific delivery, this can be accomplished, for example, in a passive manner wherein the liposome bilayer degrades over time through the action of various agents in the body. Alternatively, active drug release involves using an agent to induce a permeability change in the liposome vesicle.

Liposome membranes can be constructed so that they become destabilized when the environment becomes acidic near the liposome membrane (see, e.g., PNAS. 84:7851 (1987); Biochemistry. 28:908 (1989)). When liposomes are endocytosed by a target cell, for example, they become destabilized and release their contents. This destabilization is termed fusogenesis. Dioleoylphosphatidylethanolamine (DOPE) is the basis of many “fusogenic” systems.

Such liposomes typically comprise a targeted transcriptional effector and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes as described in, e.g., Szoka et al, Ann. Rev. Biophys. Bioeng 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication WO 91/17424, Deamer & Bangham, Biochim. Biophys. Acta. 443:629-634 (1976); Fraley, et al., PNAS. 76:3348-3352 (1979); Hope et al., Biochim. Biophys. Acta. 812:55-65 (1985); Mayer et al., Biochim. Biopllys. Acta. 858:161-168 (1986); Williams et al., PNAS. 85:242-246 (1988); Liposomes (Ostro (ed.), 1983, Chapter 1); Hope et al., Chem. Phys. Lip. 40:89 (1986); Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments, it is desirable to target liposomes using targeting moieties that are specific to a particular cell type, tissue, and the like. Targeting of liposomes using a variety of targeting moieties (e.g., ligands, receptors, and monoclonal antibodies) has been previously described (see, e.g., U.S. Pat. Nos. 4,957,773 and 4,603,044).

Examples of targeting moieties include monoclonal antibodies specific to antigens associated with neoplasms, such as prostate cancer specific antigen and MAGE. Tumors can also be diagnosed by detecting gene products resulting from the activation or over-expression of oncogenes, such as ras or c-erbB2. In addition, many tumors express antigens normally expressed by fetal tissue, such as the alphafetoprotein (AFP) and carcinoembryonic antigen (CEA). Sites of viral infection can be diagnosed using various viral antigens such as hepatitis B core and surface antigens (HBVc, HBVs) hepatitis C antigens, Epstein-Barr virus antigens, human immunodeficiency type-1 virus (HIV1) and papilloma virus antigens. Inflammation can be detected using molecules specifically recognized by surface molecules which are expressed at sites of inflammation such as integrins (e.g., VCAM-1), selectin receptors (e.g., ELAM-1) and the like.

Standard methods for coupling targeting agents to liposomes can be used. These methods generally involve incorporation into liposomes lipid components, e.g., phosphatidylethanolamine, which can be activated for attachment of targeting agents, or derivatized lipophilic compounds, such as lipid derivatized bleomycin. Antibody targeted liposomes can be constructed using, for instance, liposomes which incorporate protein A (see Renneisen et al., J. Biol. Chem. 265:16337-16342 (1990) and Leonetti et al., PNAS. 87:2448-2451 (1990).

### 15. Dosages

For therapeutic applications, the dose administered to a patient, in the context of the present disclosure, should be sufficient to effect a beneficial therapeutic response in the patient over time. In addition, particular dosage regimens can be useful for determining phenotypic changes in an experimental setting, e.g., in functional genomics studies, and in cell or animal models. The dose will be determined by the efficacy and K_{d} of the particular engineered DNS-binding domain employed, the nuclear volume of the target cell, and the condition of the patient, as well as the body weight or surface area of the patient to be treated. The size of the dose also will be determined by the existence, nature, and extent of any adverse side-effects that accompany the administration of a particular compound or vector in a particular patient.

The maximum therapeutically effective dosage of targeted transcriptional effector for approximately 99% binding to target sites is calculated to be in the range of less than about 1. 5×10⁵ to 1. 5×10⁶ copies of the specific targeted transcriptional effector molecule per cell. The number of targeted transcriptional effector s per cell for this level of binding is calculated as follows, using the volume of a HeLa cell nucleus (approximately 1000 μm³ or 10⁻¹² L; Cell Biology, (Altman & Katz, eds. (1976)). As the HeLa nucleus is relatively large, this dosage number is recalculated as needed using the volume of the target cell nucleus. This calculation also does not take into account competition for targeted transcriptional effector binding by other sites. This calculation also assumes that essentially all of the targeted transcriptional effector is localized to the nucleus. A value of 100×K_{d} is used to calculate approximately 99% binding of to the target site, and a value of 10×K_{d} is used to calculate approximately 90% binding of to the target site.

The appropriate dose of an expression vector encoding a targeted transcriptional effector can also be calculated by taking into account the average rate of targeted transcriptional effector expression from the promoter and the average rate of targeted transcriptional effector degradation in the cell. A weak promoter such as a wild-type or mutant HSV TK can be used, as described above. The dose of targeted transcriptional effector in micrograms is calculated by taking into account the molecular weight of the particular targeted transcriptional effector being employed.

In determining the effective amount of the targeted transcriptional effector to be administered in the treatment or prophylaxis of disease, the physician evaluates circulating plasma levels of the targeted transcriptional effector or nucleic acid encoding the targeted transcriptional effector, potential targeted transcriptional effector toxicities, progression of the disease, and the production of anti-targeted transcriptional effector antibodies. Administration can be accomplished via single or divided doses.

### 16. Pharmaceutical Compositions and Administration

Targeted transcriptional effector s and expression vectors encoding targeted transcriptional effectors can be administered directly to the patient for modulation of gene expression and for therapeutic or prophylactic applications, for example, cancer, ischemia, diabetic retinopathy, macular degeneration, rheumatoid arthritis, psoriasis, HIV infection, sickle cell anemia, Alzheimer's disease, muscular dystrophy, neurodegenerative diseases, vascular disease, cystic fibrosis, stroke, and the like. Examples of microorganisms that can be inhibited by targeted transcriptional effector gene therapy include pathogenic bacteria, e.g., chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme disease bacteria; infectious fungus, e.g., Aspergillus, Candida species; protozoa such as sporozoa (e.g., Plasmodia), rhizopods (e.g., Entamoeba) and flagellates (Tijpanosoma, Leishmania, Trichonaonas, Giardia, etc.); viral diseases, e.g., hepatitis (A, B, or C), herpes virus (e.g., VZV, HSV-1, HSV-6, HSV-11, CMV, and EBV), HIV, Ebola, adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, poliovirus, rabies virus, and arboviral encephalitis virus, etc.

Administration of therapeutically effective amounts is by any of the routes normally used for introducing targeted transcriptional effector into ultimate contact with the tissue to be treated. The targeted transcriptional effectors are administered in any suitable manner, optionally with pharmaceutically acceptable carriers. Suitable methods of administering such modulators are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions that are available (see, e.g., Remisagton's Pharmaceutical Sciences, 1 7th ed. 1985)).

The targeted transcriptional effectors, alone or in combination with other suitable components, can be made into aerosol formulations (i.e., they can be “nebulized”) to be administered via inhalation.

Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

Regulation of gene expression in plants targeted transcriptional effectors can be used to engineer plants for traits such as increased disease resistance, modification of structural and storage polysaccharides, flavors, proteins, and fatty acids, fruit ripening, yield, color, nutritional characteristics, improved storage capability, and the like. In particular, the engineering of crop species for enhanced oil production, e.g., the modification of the fatty acids produced in oilseeds, is of interest.

Seed oils are composed primarily of triacylglycerols (TAGs), which are glycerol esters of fatty acids. Commercial production of these vegetable oils is accounted for primarily by six major oil crops (soybean, oil palm, rapeseed, sunflower, cotton seed, and peanut.) Vegetable oils are used predominantly (90%) for human consumption as margarine, shortening, salad oils, and frying oil. The remaining 10% is used for non-food applications such as lubricants, oleochemicals, biofuels, detergents, and other industrial applications.

The desired characteristics of the oil used in each of these applications varies widely, particularly in terms of the chain length and number of double bonds present in the fatty acids making up the TAGs. These properties are manipulated by the plant in order to control membrane fluidity and temperature sensitivity. The same properties can be controlled using targeted transcriptional effectors to produce oils with improved characteristics for food and industrial uses.

The primary fatty acids in the TAGs of oilseed crops are 16 to 18 carbons in length and contain 0 to 3 double bonds. Palmitic acid (16:0 [16 carbons: 0 double bonds]), oleic acid (18:1), linoleic acid (18:2), and linolenic acid (18:3) predominate. The number of double bonds, or degree of saturation, determines the melting temperature, reactivity, cooking performance, and health attributes of the resulting oil.

The enzyme responsible for the conversion of oleic acid (18:1) into linoleic acid (18:2) (which is then the precursor for 18:3 formation) is A12-oleate desaturase, also referred to as omega-6 desaturase. A block at this step in the fatty acid desaturation pathway should result in the accumulation of oleic acid at the expense of polyunsaturates.

In one embodiment targeted transcriptional effectors are used to regulate expression of the FAD2-1 gene in soybeans. Two genes encoding microsomal A6 desaturases have been cloned recently from soybean, and are referred to as FAD2-1 and FAD2-2 (Heppard et al., Plant Physiol. 110:311-319 (1996)). FAD2-1 (delta 12 desaturase) appears to control the bulk of oleic acid desaturation in the soybean seed. Targeted transcriptional effectors can thus be used to modulate gene expression of FAD2-1 in plants. Specifically, targeted transcriptional effectors can be used to inhibit expression of the FAD2-1 gene in soybean in order to increase the accumulation of oleic acid (18:1) in the oil seed. Moreover, targeted transcriptional effectors can be used to modulate expression of any other plant gene, such as delta-9 desaturase, delta-12 desaturases from other plants, delta-15 desaturase, acetyl-CoA carboxylase, acyl-ACP-thioesterase, ADP-glucose pyrophosphorylase, starch synthase, cellulose synthase, sucrose synthase, senescence-associated genes, heavy metal chelators, fatty acid hydroperoxide lyase, polygalacturonase, EPSP synthase, plant viral genes, plant fungal pathogen genes, and plant bacterial pathogen genes.

Recombinant DNA vectors suitable for transformation of plant cells are also used to deliver protein (e.g., targeted transcriptional effector)-encoding nucleic acids to plant cells. Techniques for transforming a wide variety of higher plant species are well known and described in the technical and scientific literature (see, e.g., Weising et al. Ann. Rev. Genet. 22:421-477 (1988)). A DNA sequence coding for the desired targeted transcriptional effectors is combined with transcriptional and translational initiation regulatory sequences which will direct the transcription of the targeted transcriptional effectors in the intended tissues of the transformed plant.

For example, a plant promoter fragment may be employed which will direct expression of the targeted transcriptional effectors in all tissues of a regenerated plant. Such promoters are referred to herein a “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacteriuna turnafaciefas, and other transcription initiation regions from various plant genes known to those of skill.

Alternatively, the plant promoter may direct expression of the targeted transcriptional effectors in a specific tissue or may be otherwise under more precise environmental or developmental control.

Such promoters are referred to here as “inducible” promoters. Examples of environmental conditions that may effect transcription by inducible promoters include anaerobic conditions or the presence of light.

Examples of promoters under developmental control include promoters that initiate transcription only in certain tissues, such as fruit, seeds, or flowers. For example, the use of a polygalacturonase promoter can direct expression of the targeted transcriptional effectors in the fruit, a CHS-A (chalcone synthase A from petunia) promoter can direct expression of the ZFP in flower of a plant.

The vector comprising a targeted transcriptional effector sequence will typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

Such DNA constructs may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using biolistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al. EMBO J. 3:2717-2722 (1984).

Electroporation techniques are described in Fromm et al. PNAS. 82:5824 (1985). Biolistic transformation techniques are described in Klein et al. Nature. 327:70-73 (1987).

Agrobacterium tumefaciens-meditated transformation techniques are well described in the scientific literature (see, e.g., Horsch et al. Science. 233:496-498 (1984); and Fraley et al. PNAS. 80:4803 (1983)).

Transformed plant cells which are derived by any of the above transformation techniques can be cultured to regenerate a whole plant which possesses the transformed genotype and thus the desired targeted transcriptional effector-controlled phenotype. Such regeneration techniques rely on manipulation of certain phytohormones in a tissue culture growth medium, typically relying on a biocide and/or herbicide marker which has been introduced together with the ZFP nucleotide sequences. Plant regeneration from cultured protoplasts is described in Evans et al., Protoplasts Isolation and Culture, Handbook of Plant Cell Culture, pp. 124-176 (1983); and Binding, Regeneration of Plants, Plant Protoplasts, pp. 21-73 (1985). Regeneration can also be obtained from plant callus, explants, organs, or parts thereof. Such regeneration techniques are described generally in Klee et al. Ann. Rev. of Plant Plays. 38:467-486 (1987).

Functional genomics assays targeted transcriptional effectors also have use for assays to determine the phenotypic consequences and function of gene expression. The recent advances in analytical techniques, coupled with focused mass sequencing efforts have created the opportunity to identify and characterize many more molecular targets than were previously available. This new information about genes and their functions will speed along basic biological understanding and present many new targets for therapeutic intervention. In some cases analytical tools have not kept pace with the generation of new data. An example is provided by recent advances in the measurement of global differential gene expression.

These methods, typified by gene expression microarrays, differential cDNA cloning frequencies, subtractive hybridization and differential display methods, can very rapidly identify genes that are up or down-regulated in different tissues or in response to specific stimuli. Increasingly, such methods are being used to explore biological processes such as, transformation, tumor progression, the inflammatory response, neurological disorders etc. One can now very easily generate long lists of differentially expressed genes that correlate with a given physiological phenomenon, but demonstrating a causative relationship between an individual differentially expressed gene and the phenomenon is difficult. Until now, simple methods for assigning function to differentially expressed genes have not kept pace with the ability to monitor differential gene expression.

Using conventional molecular approaches, over expression of a candidate gene can be accomplished by cloning a full-length cDNA, subcloning it into a mammalian expression vector and transfecting the recombinant vector into an appropriate host cell.

This approach is straightforward but labor intensive, particularly when the initial candidate gene is represented by a simple expressed sequence tag (EST). Under expression of a candidate gene by “conventional” methods is yet more problematic.

Antisense methods and methods that rely on targeted ribozymes are unreliable, succeeding for only a small fraction of the targets selected. Gene knockout by homologous recombination works fairly well in recombinogenic stem cells but very inefficiently in somatically derived cell lines. In either case large clones of syngeneic genomic DNA (on the order of 10 kb) should be isolated for recombination to work efficiently.

The targeted transcriptional effectors technology can be used to rapidly analyze differential gene expression studies. Engineered targeted transcriptional effectors can be readily used to up or down-regulate any endogenous target gene. Very little sequence information is required to create a gene-specific DNA binding domain. This makes the targeted transcriptional effectors technology ideal for analysis of long lists of poorly characterized differentially expressed genes. One can simply build a zinc finger-based DNA binding domain for each candidate gene, create chimeric up and down-regulating artificial transcription factors and test the consequence of up or down-regulation on the phenotype under study (transformation, response to a cytokine etc.) by switching the candidate genes on or off one at a time in a model system.

This specific example of using engineered targeted transcriptional effectors s to add functional information to genomic data is merely illustrative. Any experimental situation that could benefit from the specific up or down-regulation of a gene or genes could benefit from the reliability and ease of use of engineered targeted transcriptional effectors.

Additionally, greater experimental control can be imparted by targeted transcriptional effectors than can be achieved by more conventional methods. This is because the production and/or function of an engineered targeted transcriptional effectors can be placed under small molecule control. Examples of this approach are provided by the Tet-On system, the ecdysone-regulated system and a system incorporating a chimeric factor including a mutant progesterone receptor. These systems are all capable of indirectly imparting small molecule control on any endogenous gene of interest or any transgene by placing the function and/or expression of a targeted transcriptional effectors regulator under small molecule control.

### 17. Transgenic Animals

A further application of the targeted transcriptional effector technology is manipulating gene expression in transgenic animals. As with cell lines, over-expression of an endogenous gene or the introduction of a heterologous gene to a transgenic animal, such as a transgenic mouse, is a fairly straightforward process. The targeted transcriptional effector technology is an improvement in these types of methods because one can circumvent the need for generating full-length cDNA clones of the gene under study.

Likewise, as with cell-based systems, conventional down-regulation of gene expression in transgenic animals is plagued by technical difficulties. Gene knockout by homologous recombination is the method most commonly applied currently. This method requires a relatively long genomic clone of the gene to be knocked out (ca. 10 kb). Typically, a selectable marker is inserted into an exon of the gene of interest to effect the gene disruption, and a second counter-selectable marker provided outside of the region of homology to select homologous versus non-homologous recombinants. This construct is transfected into embryonic stem cells and recombinants selected in culture.

Recombinant stem cells are combined with very early stage embryos generating chimeric animals. If the chimerism extends to the germline homozygous knockout animals can be isolated by back-crossing. When the technology is successfully applied, knockout animals can be generated in approximately one year. Unfortunately two common issues often prevent the successful application of the knockout technology; embryonic lethality and developmental compensation. Embryonic lethality results when the gene to be knocked out plays an essential role in development. This can manifest itself as a lack of chimerism, lack of germline transmission or the inability to generate homozygous back crosses. Genes can play significantly different physiological roles during development versus in adult animals. Therefore, embryonic lethality is not considered a rationale for dismissing a gene target as a useful target for therapeutic intervention in adults.

Embryonic lethality most often simply means that the gene of interest can not be easily studied in mouse models, using conventional methods.

Developmental compensation is the substitution of a related gene product for the gene product being knocked out. Genes often exist in extensive families. Selection or induction during the course of development can in some cases trigger the substitution of one family member for another mutant member. This type of functional substitution may not be possible in the adult animal. A typical result of developmental compensation would be the lack of a phenotype in a knockout mouse when the ablation of that gene's function in an adult would otherwise cause a physiological change. This is a kind of false negative result that often confounds the interpretation of conventional knockout mouse models.

A few new methods have been developed to avoid embryonic lethality. These methods are typified by an approach using the cre recombinase and lox DNA recognition elements. The recognition elements are inserted into a gene of interest using homologous recombination (as described above) and the expression of the recombinase induced in adult mice post-development. This causes the deletion of a portion of the target gene and avoids developmental complications. The method is labor intensive and suffers form chimerism due to non-uniform induction of the recombinase.

The use of targeted transcriptional effectors to manipulate gene expression can be restricted to adult animals using the small molecule regulated systems described in the previous section. Expression and/or function of a zinc finger-based repressor can be switched off during development and switched on at will in the adult animals. This approach relies on the addition of the targeted transcriptional effectors expressing module only; homologous recombination is not required. Because the targeted transcriptional effectors repressors are trans dominant, there is no concern about germline transmission or homozygosity. These issues dramatically affect the time and labor required to go from a poorly characterized gene candidate (a cDNA or EST clone) to a mouse model. This ability can be used to rapidly identify and/or validate gene targets for therapeutic intervention, generate novel model systems and permit the analysis of complex physiological phenomena (development, hematopoiesis, transformation, neural function etc.). Chimeric targeted mice can be derived according to Hogan et al., Manipulating the Mouse Embryo: A Laboratory Manual, (1988); Teratocarcinomas and Embryonic Stem Cells: A Practical Approach, Robertson, ed., Oxford University Press (1987); and Capecchi et al., Science. 244:1288 (1989).

### EXAMPLES

Embodiments of the invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below. Examples 1˜4 below refer specifically to non-naturally-occurring, rationally-designed meganucleases based on I-CreI, but non-naturally-occurring, rationally-designed meganucleases based on I-SceI, I-MsoI, I-CeuI, and other LAGLIDADG meganucleases can be similarly produced and used, as described herein.

### Example 1

### Rational Design of Meganucleases Recognizing the HIV-1 TAT Gene

### 1. Rational Meganuclease Design.

A pair of meganucleases were rationally-designed to recognize and cleave the DNA site 5′-GAAGAGCTCATCAGAACAGTCA-3′ (SEQ ID NO: 15) found in the HIV-1 TAT Gene. In accordance with Table 1, two meganucleases, TAT1 and TAT2, were designed to bind the half-sites 5′-GAAGAGCTC-3′ (SEQ ID NO: 16) and 5′-TGACTGTTC-3′ (SEQ ID NO: 17), respectively, using the following base contacts (non-WT contacts are in bold):

TAT1:

| Position | −9 | −8 | −7 | −6 | −5 | −4 | −3 | −2 | −1 |
|---|---|---|---|---|---|---|---|---|---|
| Base | G | A | A | G | A | G | C | T | C |
| Contact Residues | S32 | Y33 | N30/ | R40 | K28 | S26/ | K24/ | Q44 | R70 |
| | | | Q38 | | | R77 | Y68 | | |

TAT2:

| Position | −9 | −8 | −7 | −6 | −5 | −4 | −3 | −2 | −1 |
|---|---|---|---|---|---|---|---|---|---|
| Base | T | G | A | C | T | G | T | T | C |
| Contact Residues | C32 | R33 | N30/ | R28/ | M66 | S26/ | Y68 | Q44 | R70 |
| | | | Q38 | E40 | | R77 | | | |

The two enzymes were cloned, expressed in E. coli, and assayed for enzyme activity against the corresponding DNA recognition sequence as described below. In both cases, the rationally-designed meganucleases were found to be inactive. A second generation of each was then produced in which E80 was mutated to Q to improve contacts with the DNA backbone. The second generation TAT2 enzyme was found to be active against its intended recognition sequence while the second generation TAT1 enzyme remained inactive. Visual inspection of the wild-type I-CreI co-crystal structure suggested that TAT1 was inactive due to a steric clash between R40 and K28. To alleviate this clash, TAT1 variants were produced in which K28 was mutated to an amino acid with a smaller side chain (A, S, T, or C) while maintaining the Q80 mutation. When these enzymes were produced in E. coli and assayed, the TAT1 variants with S28 and T28 were both found to be active against the intended recognition sequence while maintaining the desired base preference at position −7.

### 2. Construction of Recombinant Meganucleases.

Mutations for the redesigned I-CreI enzymes were introduced using mutagenic primers in an overlapping PCR strategy. Recombinant DNA fragments of I-CreI generated in a primary PCR were joined in a secondary PCR to produce full-length recombinant nucleic acids. All recombinant I-CreI constructs were cloned into pET21a vectors with a six histidine tag fused at the 3′ end of the gene for purification (Novagen Corp., San Diego, CA). All nucleic acid sequences were confirmed using Sanger Dideoxynucleotide sequencing (see Sanger et al. (1977), Proc. Natl. Acad. Sci. USA. 74 (12): 5463-7).

Wild-type I-CreI and all engineered meganucleases were expressed and purified using the following method. The constructs cloned into a pET21a vector were transformed into chemically competent BL21 (DE3) pLysS, and plated on standard 2×YT plates containing 200 μg/ml carbanicillin. Following overnight growth, transformed bacterial colonies were scraped from the plates and used to inoculate 50 ml of 2×YT broth. Cells were grown at 37° C. with shaking until they reached an optical density of 0.9 at a wavelength of 600 nm. The growth temperature was then reduced from 37° C. to 22° C. Protein expression was induced by the addition of 1 mM IPTG, and the cells were incubated with agitation for two and a half hours. Cells were then pelleted by centrifugation for 10 min. at 6000×g. Pellets were resuspended in 1 ml binding buffer (20 mM Tris-HCL, pH 8.0, 500 mM NaCl, 10 mM imidazole) by vortexing. The cells were then disrupted with 12 pulses of sonication at 50% power and the cell debris was pelleted by centrifugation for 15 min. at 14,000×g. Cell supernatants were diluted in 4 ml binding buffer and loaded onto a 200 μl nickel-charged metal-chelating Sepharose column (Pharmacia).

The column was subsequently washed with 4 ml wash buffer (20 mM Tris-HCl, pH 8.0, 500 mM NaCl, 60 mM imidazole) and with 0.2 ml elution buffer (20 mM Tris-HCl, pH 8.0, 500 mM NaCl, 400 mM imidazole). Meganuclease enzymes were eluted with an additional 0.6 ml of elution buffer and concentrated to 50-130 μl using Vivospin disposable concentrators (ISC, Inc., Kaysville, UT). The enzymes were exchanged into SA buffer (25 mM Tris-HCL, pH 8.0, 100 mM NaCl, 5 mM MgCl₂, 5 mM EDTA) for assays and storage using Zeba spin desalting columns (Pierce Biotechnology, Inc., Rockford, IL). The enzyme concentration was determined by absorbance at 280 nm using an extinction coefficient of 23,590 M⁻¹ cm⁻¹. Purity and molecular weight of the enzymes was then confirmed by MALDI-TOF mass spectrometry.

Heterodimeric enzymes were produced either by purifying the two proteins independently, and mixing them in vitro or by constructing an artificial operon for tandem expression of the two proteins in E. coli. In the former case, the purified meganucleases were mixed 1:1 in solution and pre-incubated at 42° C. for 20 minutes prior to the addition of DNA substrate. In the latter case, the two genes were cloned sequentially into the pET-21a expression vector using NdeI/EcoRI and EcoRI/HindIII. The first gene in the operon ends with two stop codons to prevent read-through errors during transcription. A 12-base pair nucleic acid spacer and a Shine-Dalgarno sequence from the pET21 vector separated the first and second genes in the artificial operon.

### 3. Cleavage Assays.

All enzymes purified as described above were assayed for activity by incubation with linear, double-stranded DNA substrates containing the meganuclease recognition sequence. Synthetic oligonucleotides corresponding to both sense and antisense strands of the recognition sequence were annealed and were cloned into the Smal site of the pUC19 plasmid by blunt-end ligation. The sequences of the cloned binding sites were confirmed by Sanger dideoxynucleotide sequencing. All plasmid substrates were linearized with XmnI, ScaI or BpmI concurrently with the meganuclease digest. The enzyme digests contained 5 μl 0.05 μM DNA substrate, 2.5 μl 5 μM recombinant I-CreI meganuclease, 9.5 μl SA buffer, and 0.5 μl XmnI, ScaI, or BpmI. Digests were incubated at either 37° C., or 42° C. for certain meganuclease enzymes, for four hours. Digests were stopped by adding 0.3 mg/ml Proteinase K and 0.5% SDS, and incubated for one hour at 37° C. Digests were analyzed on 1.5% agarose and visualized by ethidium bromide staining.

To evaluate meganuclease half-site preference, rationally-designed meganucleases were incubated with a set of DNA substrates corresponding to a perfect palindrome of the intended half-site as well as each of the 27 possible single-base-pair substitutions in the half-site. In this manner, it was possible to determine how tolerant each enzyme is to deviations from its intended half-site.

### 4. Recognition Sequence-Specificity.

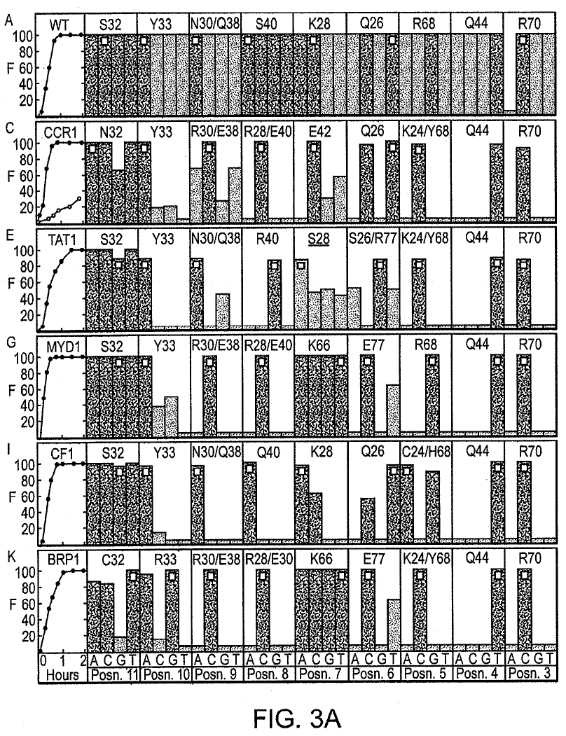

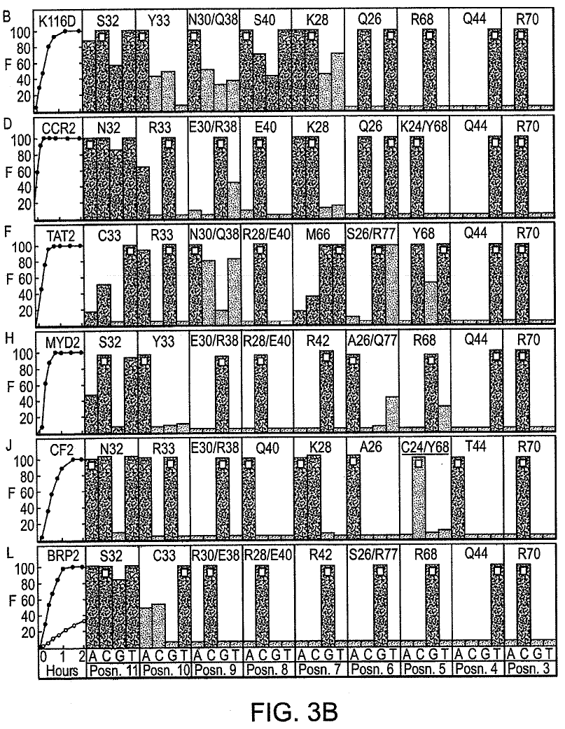

Purified recombinant TAT1 and TAT2 meganucleases recognized DNA sequences that were distinct from the wild-type meganuclease recognition sequence (FIG. 2(B)). The wild-type I-CreI meganuclease cleaves the WT recognition sequence, but cuts neither the intended sequence for TAT1 nor the intended sequence for TAT2. TAT1 and TAT2, likewise, cut their intended recognition sequences but not the wild-type sequence. The meganucleases were then evaluated for half-site preference and overall specificity (FIG. 3). Wild-type I-CreI was found to be highly tolerant of single-base-pair substitutions in its natural half-site. In contrast, TAT1 and TAT2 were found to be highly-specific and completely intolerant of base substitutions at positions −1, −2, −3, −6, and −8 in the case of TAT1, and positions −1, −2, and −6 in the case of TAT2.

### Example 2

### Rational Design of Meganucleases with Altered DNA-Binding Affinity

1. Rationally-Designed Meganucleases with Increased Affinity and Increased Activity.

The meganucleases CCR1 and BRP2 were rationally-designed to cleave the half-sites 5′-AACCCTCTC-3′ (SEQ ID NO: 18) and 5′-CTCCGGGTC-3′ (SEQ ID NO: 19), respectively. These enzymes were produced in accordance with Table 1 as in Example 1:

CCR1:

| Position | −9 | −8 | −7 | −6 | −5 | −4 | −3 | −2 | −1 |
|---|---|---|---|---|---|---|---|---|---|
| Base | A | A | C | C | C | T | C | T | C |
| Contact Residues | N32 | Y33 | R30/ | R28/ | E42 | Q26 | K24/ | Q44 | R70 |
| | | | E38 | E40 | | | Y68 | | |

BRP2:

| Position | −9 | −8 | −7 | −6 | −5 | −4 | −3 | −2 | −1 |
|---|---|---|---|---|---|---|---|---|---|
| Base | C | T | C | C | G | G | G | T | C |
| Contact Residues | S32 | C33 | R30/ | R28/ | R42 | S26/ | R68 | Q44 | R70 |
| | | | E38 | E40 | | R77 | | | |

Both enzymes were expressed in E. coli, purified, and assayed as in Example 1. Both first generation enzymes were found to cleave their intended recognition sequences with rates that were considerably below that of wild-type I-CreI with its natural recognition sequence. To alleviate this loss in activity, the DNA-binding affinity of CCR1 and BRP2 was increased by mutating E80 to Q in both enzymes. These second-generation versions of CCR1 and BRP2 were found to cleave their intended recognition sequences with substantially increased catalytic rates.

2. Rationally-Designed Meganucleases with Decreased DNA-Binding Affinity and Decreased Activity but Increased Specificity.

Wild-type I-CreI was found to be highly-tolerant of substitutions to its half-site (FIG. 3(A)). In an effort to make the enzyme more specific, the lysine at position 116 of the enzyme, which normally makes a salt-bridge with a phosphate in the DNA backbone, was mutated to aspartic acid to reduce DNA-binding affinity. This rationally-designed enzyme was found to cleave the wild-type recognition sequence with substantially reduced activity but the recombinant enzyme was considerably more specific than wild-type. The half-site preference of the K116D variant was evaluated as in Example 1 and the enzyme was found to be entirely intolerant of deviation from its natural half-site at positions −1, −2, and −3, and displayed at least partial base preference at the remaining 6 positions in the half-site (FIG. 3(B)).

### Example 3

### Rationally-Designed Meganuclease Heterodimers

### 1. Cleavage of Non-Palindromic DNA Sites by Rationally-Designed Meganuclease Heterodimers Formed in Solution.

Two meganucleases, LAM1 and LAM2, were rationally-designed to cleave the half-sites 5′-TGCGGTGTC-3′ (SEQ ID NO: 20) and 5′-CAGGCTGTC-3′ (SEQ ID NO: 21), respectively. The heterodimer of these two enzymes was expected to recognize the DNA sequence 5′-TGCGGTGTCCGGCGACAGCCTG-3′ (SEQ ID NO: 22) found in the bacteriophage λ p05 gene.

LAM1:

| Position | −9 | −8 | −7 | −6 | −5 | −4 | −3 | −2 | −1 |
|---|---|---|---|---|---|---|---|---|---|
| Base | T | G | C | G | G | T | G | T | C |
| Contact Residues | C32 | R33 | R30/ | D28/ | R42 | Q26 | R68 | Q44 | R70 |
| | | | E38 | R40 | | | | | |

LAM2:

| Position | −9 | −8 | −7 | −6 | −5 | −4 | −3 | −2 | −1 |
|---|---|---|---|---|---|---|---|---|---|
| Base | C | A | G | G | C | T | G | T | C |
| Contact Residues | S32 | Y33 | E30/ | R40 | K28/ | Q26 | R68 | Q44 | R70 |
| | | | R38 | | E42 | | | | |

LAM1 and LAM 2 were cloned, expressed in E. coli, and purified individually as described in Example 1. The two enzymes were then mixed 1:1 and incubated at 42° C. for 20 minutes to allow them to exchange subunits and re-equilibrate. The resulting enzyme solution, expected to be a mixture of LAM1 homodimer, LAM2 homodimer, and LAM1/LAM2 heterodimer, was incubated with three different recognition sequences corresponding to the perfect palindrome of the LAM1 half-site, the perfect palindrome of the LAM2 half-site, and the non-palindromic hybrid site found in the bacteriophage λ genome. The purified LAM1 enzyme alone cuts the LAM1 palindromic site, but neither the LAM2 palindromic site, nor the LAM1/LAM2 hybrid site. Likewise, the purified LAM2 enzyme alone cuts the LAM2 palindromic site but neither the LAM1 palindromic site nor the LAM1/LAM2 hybrid site. The 1:1 mixture of LAM1 and LAM2, however, cleaves all three DNA sites. Cleavage of the LAM1/LAM2 hybrid site indicates that two distinct re-designed meganucleases can be mixed in solution to form a heterodimeric enzyme capable of cleaving a non-palindromic DNA site.

### 2. Cleavage of Non-Palindromic DNA Sites by Meganuclease Heterodimers Formed by Co-Expression.

Genes encoding the LAM1 and LAM2 enzymes described above were arranged into an operon for simultaneous expression in E. coli as described in Example 1. The co-expressed enzymes were purified as in Example 1 and the enzyme mixture incubated with the three potential recognition sequences described above. The co-expressed enzyme mixture was found to cleave all three sites, including the LAM1/LAM2 hybrid site, indicating that two distinct rationally-designed meganucleases can be co-expressed to form a heterodimeric enzyme capable of cleaving a non-palindromic DNA site.

3. Preferential Cleavage of Non-Palindromic DNA Sites by Meganuclease Heterodimers with Modified Protein-Protein Interfaces.

For applications requiring the cleavage of non-palindromic DNA sites, it is desirable to promote the formation of enzyme heterodimers while minimizing the formation of homodimers that recognize and cleave different (palindromic) DNA sites. To this end, variants of the LAM1 enzyme were produced in which lysines at positions 7, 57, and 96 were changed to glutamic acids. This enzyme was then co-expressed and purified as in above with a variant of LAM2 in which glutamic acids at positions 8 and 61 were changed to lysine. In this case, formation of the LAM1 homodimer was expected to be reduced due to electrostatic repulsion between E7, E57, and E96 in one monomer and E8 and E61 in the other monomer. Likewise, formation of the LAM2 homodimer was expected to be reduced due to electrostatic repulsion between K7, K57, and K96 on one monomer and K8 and K61 on the other monomer. Conversely, the LAM1/LAM2 heterodimer was expected to be favored due to electrostatic attraction between E7, E57, and E96 in LAM1 and K8 and K61 in LAM2. When the two meganucleases with modified interfaces were co-expressed and assayed as described above, the LAM1/LAM2 hybrid site was found to be cleaved preferentially over the two palindromic sites, indicating that substitutions in the meganuclease protein-protein interface can drive the preferential formation of heterodimers.

### Example 4

### Additional Rationally-Designed Meganuclease Heterodimers which Cleave Physiologic DNA Sequences

1. Rationally-Designed Meganuclease Heterodimers which Cleave DNA Sequences Relevant to Gene Therapy.

A rationally-designed meganuclease heterodimer (ACH1/ACH2) can be produced that cleaves the sequence 5′-CTGGGAGTCTCAGGACAGCCTG-3′ (SEQ ID NO: 23) in the human FGFR3 gene, mutations in which cause achondroplasia. For example, a meganuclease was rationally-designed based on the I-CreI meganuclease, as described above, with the following contact residues and recognition sequence half-sites:

ACH1:

| Position | −9 | −8 | −7 | −6 | −5 | −4 | −3 | −2 | −1 |
|---|---|---|---|---|---|---|---|---|---|
| Base | C | T | G | G | G | A | G | T | C |
| Contact Residues | D32 | C33 | E30/ | R40/ | R42 | A26/ | R68 | Q44 | R70 |
| | | | R38 | D28 | | Q77 | | | |

ACH2:

| Position | −9 | −8 | −7 | −6 | −5 | −4 | −3 | −2 | −1 |
|---|---|---|---|---|---|---|---|---|---|
| Base | C | A | G | G | C | T | G | T | C |
| Contact Residues | D32 | Y33 | E30/ | R40 | K28/ | Q26 | R68 | Q44 | R70 |
| | | | R38 | | E42 | | | | |

A rationally-designed meganuclease heterodimer (HGH1/HGH2) can be produced that cleaves the sequence 5′-CCAGGTGTCTCTGGACTCCTCC-3′ (SEQ ID NO: 24) in the promoter of the Human Growth Hormone gene. For example, a meganuclease was rationally-designed based on the I-CreI meganuclease, as described above, with the following contact residues and recognition sequence half-sites:

HGH1:

| Position | −9 | −8 | −7 | −6 | −5 | −4 | −3 | −2 | −1 |
|---|---|---|---|---|---|---|---|---|---|
| Base | C | C | A | G | G | T | G | T | C |
| Contact Residues | D32 | C33 | N30/ | R40/ | R42 | Q26 | R68 | Q44 | R70 |
| | | | Q38 | D28 | | | | | |

HGH2:

| Position | −9 | −8 | −7 | −6 | −5 | −4 | −3 | −2 | −1 |
|---|---|---|---|---|---|---|---|---|---|
| Base | G | G | A | G | G | A | G | T | C |
| Contact Residues | K32 | R33 | N30/ | R40/ | R42 | A26 | R68 | Q44 | R70 |
| | | | Q38 | D28 | | | | | |

A rationally-designed meganuclease heterodimer (CF1/CF2) can be produced that cleaves the sequence 5′-GAAAATATCATTGGTGTTTCCT-3′ (SEQ ID NO: 25) in the ΔF508 allele of the human CFTR gene. For example, a meganuclease was rationally-designed based on the I-CreI meganuclease, as described above, with the following contact residues and recognition sequence half-sites:

CF1:

| Position | −9 | −8 | −7 | −6 | −5 | −4 | −3 | −2 | −1 |
|---|---|---|---|---|---|---|---|---|---|
| Base | G | A | A | A | A | T | A | T | C |
| Contact Residues | S32 | Y33 | N30/ | Q40 | K28 | Q26 | H68/ | Q44 | R70 |
| | | | Q38 | | | | C24 | | |

CF2:

| Position | −9 | −8 | −7 | −6 | −5 | −4 | −3 | −2 | −1 |
|---|---|---|---|---|---|---|---|---|---|
| Base | A | G | G | A | A | A | C | A | C |
| Contact Residues | N32 | R33 | E30/ | Q40 | K28 | A26 | Y68/ | T44 | R70 |
| | | | R38 | | | | C24 | | |

A rationally-designed meganuclease heterodimer (CCR1/CCR2) can be produced that cleaves the sequence 5′-AACCCTCTCCAGTGAGATGCCT-3′ (SEQ ID NO: 26) in the human CCR5 gene (an HIV co-receptor). For example, a meganuclease was rationally-designed based on the I-CreI meganuclease, as described above, with the following contact residues and recognition sequence half-sites:

CCR1:

| Position | −9 | −8 | −7 | −6 | −5 | −4 | −3 | −2 | −1 |
|---|---|---|---|---|---|---|---|---|---|
| Base | A | A | C | C | C | T | C | T | C |
| Contact Residues | N32 | Y33 | R30/ | E40/ | E42 | Q26 | Y68/ | Q44 | R70 |
| | | | E38 | R28 | | | K24 | | |

CCR2:

| Position | −9 | −8 | −7 | −6 | −5 | −4 | −3 | −2 | −1 |
|---|---|---|---|---|---|---|---|---|---|
| Base | A | G | G | C | A | T | C | T | C |
| Contact Residues | N32 | R33 | E30/ | E40 | K28 | Q26 | Y68/ | Q44 | R70 |
| | | | R38 | | | | K24 | | |

A rationally-designed meganuclease heterodimer (MYD1/MYD2) can be produced that cleaves the sequence 5′-GACCTCGTCCTCCGACTCGCTG-3′ (SEQ ID NO: 27) in the 3′ untranslated region of the human DM kinase gene. For example, a meganuclease was rationally-designed based on the I-CreI meganuclease, as described above, with the following contact residues and recognition sequence half-sites:

MYD1:

| Position | −9 | −8 | −7 | −6 | −5 | −4 | −3 | −2 | −1 |
|---|---|---|---|---|---|---|---|---|---|
| Base | G | A | C | C | T | C | G | T | C |
| Contact Residues | S32 | Y33 | R30/ | E40/ | K66 | Q26/ | R68 | Q44 | R70 |
| | | | E38 | R28 | | E77 | | | |

MYD1:

| Position | −9 | −8 | −7 | −6 | −5 | −4 | −3 | −2 | −1 |
|---|---|---|---|---|---|---|---|---|---|
| Base | C | A | G | C | G | A | G | T | C |
| Contact Residues | S32 | Y33 | E30/ | E40/ | R42 | A26 | R68 | Q44 | R70 |
| | | | R38 | R28 | | Q77 | | | |

2. Rationally-Designed Meganuclease Heterodimers which Cleave DNA Sequences in Pathogen Genomes.

A rationally-designed meganuclease heterodimer (HSV1/HSV2) can be produced that cleaves the sequence 5′-CTCGATGTCGGACGACACGGCA-3′ (SEQ ID NO: 28) in the UL36 gene of Herpes Simplex Virus-1 and Herpes Simplex Virus-2. For example, a meganuclease was rationally-designed based on the I-CreI meganuclease, as described above, with the following contact residues and recognition sequence half-sites:

HSV1:

| Position | −9 | −8 | −7 | −6 | −5 | −4 | −3 | −2 | −1 |
|---|---|---|---|---|---|---|---|---|---|
| Base | C | T | C | G | A | T | G | T | C |
| Contact Residues | S32 | C33 | R30/ | R40/ | Q42/ | Q26 | R68 | Q44 | R70 |
| | | | E38 | | K28 | | | | |

HSV2:

| Position | −9 | −8 | −7 | −6 | −5 | −4 | −3 | −2 | −1 |
|---|---|---|---|---|---|---|---|---|---|
| Base | T | G | C | C | G | T | G | T | C |
| Contact Residues | C32 | R33 | R30/ | E40/ | R42 | Q26 | R68 | Q44 | R70 |
| | | | E38 | R28 | | | | | |

A rationally-designed meganuclease heterodimer (ANT1/ANT2) can be produced that cleaves the sequence 5′-ACAAGTGTCTATGGACAGTTTA-3′ (SEQ ID NO: 29) in the Bacillus anthracis genome. For example, a meganuclease was rationally-designed based on the I-CreI meganuclease, as described above, with the following contact residues and recognition sequence half-sites:

ANT1:

| Position | −9 | −8 | −7 | −6 | −5 | −4 | −3 | −2 | −1 |
|---|---|---|---|---|---|---|---|---|---|
| Base | A | C | A | A | G | T | G | T | C |
| Contact Residues | N32 | C33 | N30/ | Q40/ | R42 | Q26 | R68 | Q44 | R70 |
| | | | Q38 | A28 | | | | | |

ANT2:

| Position | −9 | −8 | −7 | −6 | −5 | −4 | −3 | −2 | −1 |
|---|---|---|---|---|---|---|---|---|---|
| Base | T | A | A | A | C | T | G | T | C |
| Contact Residues | C32 | Y33 | N30/ | Q40 | E42 | Q26 | R68 | Q44 | R70 |
| | | | Q38 | | | | | | |

A rationally-designed meganuclease heterodimer (POX1/POX2) can be produced that cleaves the sequence 5′-AAAACTGTCAAATGACATCGCA-3′ (SEQ ID NO: 30) in the Variola (smallpox) virus gp009 gene. For example, a meganuclease was designed based on the I-CreI meganuclease, as described above, with the following contact residues and recognition sequence half-sites:

POX1:

| Position | −9 | −8 | −7 | −6 | −5 | −4 | −3 | −2 | −1 |
|---|---|---|---|---|---|---|---|---|---|
| Base | A | A | A | A | C | T | G | T | C |
| Contact Residues | N32 | C33 | N30/ | Q40 | K28 | Q26 | R68 | Q44 | R70 |
| | | | Q38 | | | | | | |

POX2:

| Position | −9 | −8 | −7 | −6 | −5 | −4 | −3 | −2 | −1 |
|---|---|---|---|---|---|---|---|---|---|
| Base | T | G | C | G | A | T | G | T | C |
| Contact | C32 | R33 | R30/ | R40 | C28/ | Q26 | R68 | Q44 | R70 |
| Resi- | | | E38 | | Q42 | | | | |
| dues | | | | | | | | | |

A rationally-designed meganuclease homodimer (EBB1/EBB1) can be produced that cleaves the pseudo-palindromic sequence 5′-CGGGGTCTCGTGCGAGGCCTCC-3′ (SEQ ID NO: 31) in the Epstein-Barr Virus BALF2 gene. For example, a meganuclease was rationally-designed based on the I-CreI meganuclease, as described above, with the following contact residues and recognition sequence half-sites:

EBB1:

| Position | −9 | −8 | −7 | −6 | −5 | −4 | −3 | −2 | −1 |
|---|---|---|---|---|---|---|---|---|---|
| Base | C | G | G | G | G | T | C | T | C |
| Contact | S32 | R33 | D30/ | R40/ | R42 | Q26 | Y68/ | Q44 | R70 |
| Resi- | | | Q38 | D28 | | | K24 | | |
| dues | | | | | | | | | |

EBB1:

| Position | −9 | −8 | −7 | −6 | −5 | −4 | −3 | −2 | −1 |
|---|---|---|---|---|---|---|---|---|---|
| Base | G | G | A | G | G | C | C | T | C |
| Contact | S32 | R33 | D30/ | R40/ | R42 | Q26 | Y68/ | Q44 | R70 |
| Resi- | | | Q38 | D28 | | | K24 | | |
| dues | | | | | | | | | |

3. Rationally-Designed Meganuclease Heterodimers which Cleave DNA Sequences in Plant Genomes.

A rationally-designed meganuclease heterodimer (GLA1/GLA2) can be produced that cleaves the sequence 5′-CACTAACTCGTATGAGTCGGTG-3′ (SEQ ID NO: 32) in the Arabidopsis thalianna GL2 gene. For example, a meganuclease was rationally-designed based on the I-CreI meganuclease, as described above, with the following contact residues and recognition sequence half-sites:

GLA1:

| Position | −9 | −8 | −7 | −6 | −5 | −4 | −3 | −2 | −1 |
|---|---|---|---|---|---|---|---|---|---|
| Base | C | A | C | T | A | A | C | T | C |
| Contact | S32 | Y33 | R30/ | S40/ | K28 | A26/ | Y68/ | Q44 | R70 |
| Resi- | | | E38 | C79 | | Q77 | K24 | | |
| dues | | | | | | | | | |

GLA2:

| Position | −9 | −8 | −7 | −6 | −5 | −4 | −3 | −2 | −1 |
|---|---|---|---|---|---|---|---|---|---|
| Base | C | A | C | C | G | A | C | T | C |
| Contact | S32 | Y33 | R30/ | E40/ | R42 | A26 | Y68/ | Q44 | R70 |
| Resi- | | | E38 | R28 | | Q77 | K24 | | |
| dues | | | | | | | | | |

A rationally-designed meganuclease heterodimer (BRP1/BRP2) can be produced that cleaves the sequence 5′-TGCCTCCTCTAGAGACCCGGAG-3′ (SEQ ID NO: 33) in the Arabidopsis thalianna BP1 gene. For example, a meganuclease was rationally-designed based on the I-CreI meganuclease, as described above, with the following contact residues and recognition sequence half-sites:

BRP1:

| Position | −9 | −8 | −7 | −6 | −5 | −4 | −3 | −2 | −1 |
|---|---|---|---|---|---|---|---|---|---|
| Base | T | G | C | C | T | C | C | T | C |
| Contact | C32 | R33 | R30/ | R28/ | K66 | Q26/ | Y68/ | Q44 | R70 |
| Resi- | | | E38 | E40 | | E77 | K24 | | |
| dues | | | | | | | | | |

BRP2:

| Position | −9 | −8 | −7 | −6 | −5 | −4 | −3 | −2 | −1 |
|---|---|---|---|---|---|---|---|---|---|
| Base | C | T | C | C | G | G | G | T | C |
| Contact | S32 | C33 | R30/ | E40/ | R42 | S26 | R68 | Q44 | R70 |
| Resi- | | | E38 | R28 | | R77 | | | |
| dues | | | | | | | | | |

A rationally-designed meganuclease heterodimer (MGC1/MGC2) can be produced that cleaves the sequence 5′-TAAAATCTCTAAGGTCTGTGCA-3′ (SEQ ID NO: 34) in the Nicotiana tabacum Magnesium Chelatase gene. For example, a meganuclease was rationally-designed based on the I-CreI meganuclease, as described above, with the following contact residues and recognition sequence half-sites:

MGC1:

| Position | −9 | −8 | −7 | −6 | −5 | −4 | −3 | −2 | −1 |
|---|---|---|---|---|---|---|---|---|---|
| Base | T | A | A | A | A | T | C | T | C |
| Contact | C32 | Y33 | N30/ | Q40/ | K28 | Q26 | Y68/ | Q44 | R70 |
| Resi- | | | Q38 | | | | K24 | | |
| dues | | | | | | | | | |

MGC2:

| Position | −9 | −8 | −7 | −6 | −5 | −4 | −3 | −2 | −1 |
|---|---|---|---|---|---|---|---|---|---|
| Base | T | G | C | A | C | A | G | A | C |
| Contact | S32 | R33 | R30/ | Q40 | K28 | A26 | R68 | T44 | R70 |
| Resi- | | | E38 | | | Q77 | | | |
| dues | | | | | | | | | |

A rationally-designed meganuclease heterodimer (CYP/HGH2) can be produced that cleaves the sequence 5′-CAAGAATTCAAGCGAGCATTAA-3′ (SEQ ID NO: 35) in the Nicotiana tabacum CYP82E4 gene. For example, a meganuclease was rationally-designed based on the I-CreI meganuclease, as described above, with the following contact residues and recognition sequence half-sites:

CYP:

| Position | −9 | −8 | −7 | −6 | −5 | −4 | −3 | −2 | −1 |
|---|---|---|---|---|---|---|---|---|---|
| Base | C | A | A | G | A | A | T | T | C |
| Contact | D32 | Y33 | N30/ | R40/ | K28 | Q77/ | Y68 | Q44 | R70 |
| Resi- | | | Q38 | | | A26 | | | |
| dues | | | | | | | | | |

HGH2:

| Position | −9 | −8 | −7 | −6 | −5 | −4 | −3 | −2 | −1 |
|---|---|---|---|---|---|---|---|---|---|
| Base | T | T | A | A | T | G | C | T | C |
| Contact | S32 | C33 | N30/ | Q40 | K66 | R77/ | Y68 | Q44 | R70 |
| Resi- | | | Q38 | | | S26 | K24 | | |
| dues | | | | | | | | | |

4. Rationally-Designed Meganuclease Heterodimers which Cleave DNA Sequences in Yeast Genomes.

A rationally-designed meganuclease heterodimer (URA1/URA2) can be produced that cleaves the sequence 5′-TTAGATGACAAGGGAGACGCAT-3′ (SEQ ID NO: 36) in the Saccharomyces cerevisiae URA3 gene. For example, a meganuclease was rationally-designed based on the I-CreI meganuclease, as described above, with the following contact residues and recognition sequence half-sites:

URA1:

| Position | −9 | −8 | −7 | −6 | −5 | −4 | −3 | −2 | −1 |
|---|---|---|---|---|---|---|---|---|---|
| Base | T | T | A | G | A | T | G | A | C |
| Contact | S32 | C33 | N30/ | R40 | K28 | Q26 | R68 | T44 | R70 |
| Resi- | | | Q38 | | | | | | |
| dues | | | | | | | | | |

URA2:

| Position | −9 | −8 | −7 | −6 | −5 | −4 | −3 | −2 | −1 |
|---|---|---|---|---|---|---|---|---|---|
| Base | A | T | G | C | G | T | C | T | C |
| Contact | N32 | C33 | E30/ | E40/ | R42 | Q26 | Y68/ | Q44 | R70 |
| Resi- | | | R38 | R28 | | | K24 | | |
| dues | | | | | | | | | |

### 5. Recognition Sequence Specificity.

The rationally-designed meganucleases outlined above in this Example were cloned, expressed in E. coli, and purified as in Example 1. Each purified meganuclease was then mixed 1:1 with its corresponding heterodimerization partner (e.g., ACH1 with ACH2, HGH1 with HGH2, etc.) and incubated with a linearized DNA substrate containing the intended non-palindromic DNA recognition sequence for each meganuclease heterodimer. As shown in FIG. 3, each rationally-designed meganuclease heterodimer cleaves its intended DNA site.

### Example 5

### Production of an Engineered DNA-Binding Domain which Recognizes a Site in the Human Genome

1. Targeting Rheumatoid Arthritis with a Targeted Transcriptional Effector.

Rheumatoid arthritis (RA) is a chronic inflammatory disease that targets synovial joints and is primarily characterize by joint destruction. The prevalence of the disease is estimated to be as high as 1% in adults and greatly diminishes the quality of life of affected individuals. Although the exact cause of the disease has yet to be determined, the immunological basis of the synovial inflammation and joint destruction is well understood. Activated monocytes and macrophages within the synovial cavity produce high levels of cytokines including interleukin-1 (IL-1) and tumor necrosis factor α (TNF-α). These pro-inflammatory cytokines induce a cascade of events that ultimately lead to the production of matrix metalloproteinases and osteoclasts, which result in severe damage to cartilage and bone.

TNF-α antagonists as therapy for RA. For decades, the only treatment options for RA were disease modifying antirheumatic drugs (DMARDs) including sulphasalazine, cyclosporine A, and methotrexate. However, several years ago, studies in animal models of inflammatory arthritis led to a new class of therapeutic agents, the TNF-α antagonists. There are currently three TNF-α antagonists available for clinical use: two are anti-TNF antibodies (Infliximab and Adalimumab) and the third is a soluble TNF-receptor fusion protein (Etanercept). These antagonists effectively block the downstream actions of TNF-α, and have demonstrated success in reducing the clinical manifestations of RA. In addition, this class of drugs is being used now to treat other conditions, including psoriasis, ankylosing spondylitis, and vasculitis. Despite the clinical success of TNF-α antagonists, there are serious adverse effects associated with these agents, including an increased risk of tuberculosis, increased incidence of lymphoma, autoimmune responses, and demyelinating syndromes. These adverse effects are likely due to the systemic inhibition of TNF-α. Given the serious nature of these side effects, there are considerable efforts to develop alternative and/or complementary strategies to treat RA and other rheumatic diseases.

Targeting TNF-α at the transcriptional level. TNF-α inhibitors currently target this important cytokine at either the protein level or the RNA level. Here, we propose to target TNF-α at the transcriptional level, by engineering a transcriptional repressor that recognizes a DNA sequence unique to the TNF-α gene. This approach has several major advantages over current tactics to inhibit TNF-α. First, by engineering a DNA-binding protein that recognizes a unique site in the TNF-α gene, the possibility of off-target effects is greatly reduced. Whereas small molecule inhibitors typically bind small motifs that may be present in multiple macromolecules, our designed DNA-binding proteins are targeted to a unique DNA sequence in the genome. Second, by aiming to reduce expression of TNF-α instead of blockading the protein entirely, our approach allows some expression of this important cytokine. By allowing baseline levels of TNF-α expression, the risk of adverse effects caused by systemic inhibition of TNF-α (with anti-TNF-α antibodies, for example) should be reduced. Third, the minimum effective dose should be significantly less for an engineered transcription factor, because there are only two copies of the TNF-α promoter in a cell and, thus, only two targets for an engineered transcription factor. For inhibitors that act at the RNA or protein level, there will be hundreds or thousands of targets which, necessarily, require high levels of inhibitors.

### 2. Production and Evaluation of the TNF_{SC} Meganuclease.

A rationally-designed meganuclease heterodimer (TNF1/TNF2) can be produced that cleaves the sequence 5′-AATGGAGACGCAAGAGAGGGAG-3′ (SEQ ID NO: 42) in the human tumor necrosis factor alpha (TNF-α) gene 436 bp downstream from the transcription start site. For example, a meganuclease was rationally-designed based on the I-CreI meganuclease, as described above, with the following contact residues and recognition sequence half-sites:

TNF1:

| Position | −9 | −8 | −7 | −6 | −5 | −4 | −3 | −2 | −1 |
|---|---|---|---|---|---|---|---|---|---|
| Base | A | A | T | G | G | A | G | A | C |
| Contact | N32 | Y33 | Q30/ | R40/ | R42 | A26/ | R68 | T44 | R70 |
| Resi- | | | S38 | D28 | | Q77 | | | |
| dues | | | | | | | | | |

TNF2:

| Position | −9 | −8 | −7 | −6 | −5 | −4 | −3 | −2 | −1 |
|---|---|---|---|---|---|---|---|---|---|
| Base | C | T | C | C | C | T | C | T | C |
| Contact | S32 | C33 | R30/ | E40/ | E42 | Q26 | Y68/ | Q44 | R70 |
| Resi- | | | E38 | R28 | | I77 | K24 | | |
| dues | | | | | | | | | |

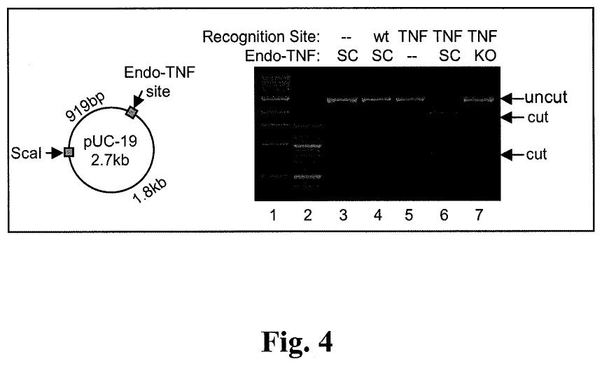

The TNF1 and TNF2 meganuclease monomers were then arranged into a single-chain meganuclease by joining an N-terminal TNF1 monomer, terminated at L155, with a C-terminal TNF2 initiated at K7 using a 38 amino acid linker (SEQ ID NO: 37). In addition, the SV40 nuclear localization signal (SEQ ID NO: 38) was added to the N-terminus. The resulting rationally-designed single-chain meganuclease is called “Endo-TNF_{SC}” (SEQ ID NO: 43). Endo-TNF_{SC} was expressed in E. coli and purified as described in Example 1. The purified meganuclease was then incubated with a plasmid substrate harboring its intended recognition sequence (SEQ ID NO: 42) and cleavage activity was determined as in Example 1. These results are shown in FIG. 4.

### 3. Production and Evaluation of the Endo-TNF_{KO} DNA-Binding Domain.

The DNA cleavage activity of Endo-TNF_{SC} was eliminated by mutating the glutamine amino acids in positions 57 and 244 to glutamic acid. Q57 and Q244 in TNF_{SC} correspond to Q47 in wild-type I-CreI. The resulting protein, Endo-TNF_{KO} (SEQ ID NO: 44), was expressed in E. coli, purified, and tested for cleavage activity as above. No DNA cleavage activity was detected (FIG. 4). Endo-TNF_{KO} was then cloned into a mammalian expression vector (pCI, Promega). This plasmid was used to transfect HEK-293 cells and binding of the Endo-TNF_{KO} protein to its intended recognition sequence in the human TNF-α gene was confirmed by chromatin immunoprecipitation using standard protocols (e.g., the protocol below).

### Chromatin Immunoprecipitation Protocol (ChIP)

1) Transfect a T-75 flask of HEK 293 cells desired plasmid using Lipofectamine 2000 according to the manufacturer's instructions.
2) 24 hours post-transfection, add 1.8 mL crosslinking mix (11% formaldehyde, 100 mM NaCl, 0.5 mM EDTA, 50 mM HEPES, pH 8.0). Incubate at room temperature for 10 minutes.
3) Quench the crosslinking reaction by adding 1.8 mL of 1.25 M glycine.
4) Remove media, and wash cells 2× with PBS.
5) Add 750 μL lysis buffer (1% SDS, 10 mM EDTA, 50 mM Tris-HCl, pH 8.0) with protease inhibitor cocktail (Sigma). Incubate at 4° C. for 5 minutes.
6) Scrape cells into a 1.5 mL Eppendorf tube.
7) Sonicate until DNA fragments approximately 500-1000 bp are generated.
8) Quantitiate protein concentration by Bradford assay.
9) Dilute 100 μg of lysates in lysis buffer to a total volume of 1 mL.
10) Pre-clear diluted lysates with 50 μL of Protein G-Sepharose beads (Sigma) for 1 hour at 4° C. with rocking.
11) Immunoprecipitate protein/DNA complexes with 10 μL Cre antisera or 10 μL FBS (fetal bovine serum) as a control. Rock overnight at 4° C.
12) Add 50 μL Protein G-Sepharose beads, and rock for 1 hour at 4°.
13) Wash beads 3× in wash buffer 1 (1% Triton X-100, 0.1% SDS, 150 mM NaCL, 2 mM EDTA, 20 mM Tris-HCl, pH 8.0) with protease inhibitors.
14) Wash beads 1× in final wash buffer (1% Triton X-100, 0.1% SDS, 500 mM NaCL, 2 mM EDTA, 20 mM Tris-HCl, pH 8.0) with protease inhibitors.
15) Wash a final time in LiCL buffer (0.25 M LiCl, 1% NP4o, 1% deoxycholate, 1 mM EDTA, 10 mM Tris-HCl, pH 8.0).
16) Elute immune complexes by adding 150 μL elution buffer (1% SDS, 100 mM NaHCO₃), Proteinase K (500 μg/mL) and RNase A (500 μg/ml) and incubating at 37° C. for 30 minutes.
17) Reverse cross-links by incubating at 65° C. for a minimum of 4 hours.
18) Recover DNA with Qiaquick spin columns. Elute in 50 μL.
19) Proceed to PCR for desired target.

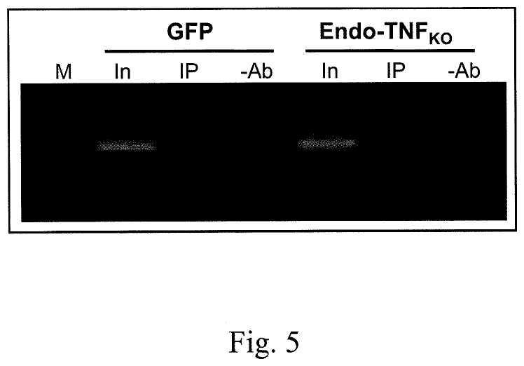

FIG. 5 shows the results of this ChIP analysis which confirms that the Endo-TNF_{KO} protein does, indeed, bind to its intended site in the TNF-α gene. Thus, Endo-TNF_{KO} is a suitable DNA-binding domain for the production of targeted transcriptional effector intended to regulate expression of the human TNF-α gene. In particular, a TNF-α repressor can be produced by linking Endo-TNF_{KO} to a KRAB repressor domain (e.g. SEQ ID NO: 41) together using a short (3-15 amino acid) linker rich in glysine and serine residues. Such a transcription factor can be delivered to human cells and its ability to repress transcription of the TNF-α gene can be determined by RT-PCR to evaluate TNF-α transcript levels or by ELISA to evaluate TNF-α protein levels.

### Example 6

### A Targeted Transcriptional Activator Derived from a Rationally Designed Meganuclease

### 1. Production of the CCR2_{KO} DNA-Binding Domain.

The DNA-contacting amino acids of the CCR2 meganuclease are presented in Example 4. The CCR2 meganuclease homodimer recognizes the palindromic DNA sequence 5′-AGGCATCTCGTACGAGATGCCT-3′ (SEQ ID NO: 45). The CCR2_{KO} meganuclease DNA-binding domain was produced by i) mutating Q47 to E (Q47E) to eliminate DNA cleavage activity ii) adding an N-terminal nuclear-localization signal (SEQ ID NO: 38).

### 2. Production of the CCR2_{REP} Engineered Transcription Factor.

A KRAB domain from the R. norvegicus Kid-1 protein (SEQ ID NO: 41) was fused to the C-terminus of CCR2_{KO} using a 9 amino acid linker (GSSGSSGSS). The resulting targeted transcriptional activator is referred to as CCR2_{REP} (SEQ ID NO 46).

3. Evaluation of CCR2_{REP} as a transcription repressor.

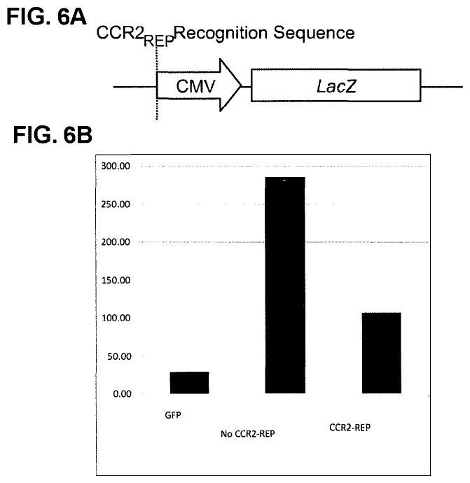

An E. coli beta-galactosidase (LacZ) gene was inserted into the mammalian expression vector pCI (Promega) between PstI and NotI. In this plasmid, LacZ expression is driven by a truncated CMV promoter (corresponding to the 3′ 442 bp of the canonical CMV promoter, SEQ ID NO: 47). A CCR2 recognition sequence (SEQ ID NO: 45) was then inserted at the 5′ end of this promoter (see FIG. 6A).

HEK 293 cells (1×10⁵) were transfected first with either the pCI empty vector or pCI carrying the CCR2_{REP} gene under the control of a constitutive CMV promoter using Lipofectamine 2000 according to the manufacturer's instructions (Invitrogen). 6 hours post-transfection, transfection complexes were removed and replaced with fresh media. 24 hours post-transfection, the cells were re-transfected with the LacZ reporter plasmid using Lipofectamine 2000. As a measure of transfection efficiency, additional cells were transfected at both time points with pCI eGFP. 24 hours post-transfection of the reporter plasmid, cells were washed with PBS, resuspended in Buffer 1 (0.01 M Tris-HCl, pH 7.9; 1 mM EDTA), lysed by sonication and clarified by centrifugation.

Lysates from transfected cells were subjected to a standard o-nitrophenyl-β-D-galactoside (ONPG) assay (Current Protocols in Molecular Biology. ed. V. B. Chanda. Vol. 2. 2004, John Wiley & Sons, Inc). Briefly, an aliquot of each lysate was diluted in 300 μL Z Buffer (60 mM Na₂HPO₄, 40 mM NaH₂PO₄, 10 mM KCl, 1 mM MgSO₄, 50 mM 2-mercaptoethanol) in 1.5 mL Eppendorf tubes. 100 μL ONPG (Sigma) was added, and the tubes were vortexed and placed in a 37° water bath. The reaction was stopped with 500 μL 1M Na₂CO₃ after one hour, and the absorbance at 420 nm was measured using a NanoDrop ND-1000 spectrophotometer. β-galactosidase activity was determined using standard equations.

The results of this experiment are shown in FIG. 6B. It was found that cells expressing CCR2_{REP} produce ˜2.6-fold less LAC-Z activity than cells transfected with the empty vector. These results indicate that a targeted transcriptional effector can be produced from a rationally-designed meganuclease.

Equivalents: Those skilled in the art will recognize, or be able to ascertain, using no more than routine experimentation, numerous equivalents to the specific embodiments described specifically herein. Such equivalents are intended to be encompassed in the scope of the following claims.

All publications and patent applications cited in this specification are herein incorporated by reference in their entireties, as if each individual publication or patent application were specifically and individually indicated to be incorporated by reference in its entirety.

1. A targeted transcriptional effector comprising: (i) an inactive meganuclease DNA-binding domain that binds to a target recognition site; and (ii) a transcription effector domain, wherein binding of the meganuclease DNA-binding domain targets the transcriptional effector to a gene of interest.

2. The targeted transcriptional effector of claim 1, further comprising a domain linker joining the meganuclease DNA-binding domain and the transcription effector domain.

3. The targeted transcriptional effector of claim 2, wherein the domain linker comprises a polypeptide.

4. The targeted transcriptional effector of claim 1, wherein the meganuclease DNA-binding domain is altered from a naturally-occurring meganuclease by at least one point mutation which reduces or abolishes endonuclease cleavage activity.

5. The targeted transcriptional effector of claim 1, further comprising a nuclear localization signal.

6. The method of claim 1, wherein the transcriptional effector domain is a transcription activator.

7. The method of claim 1, wherein the transcriptional effector domain is a transcription repressor.

8. The targeted transcriptional effector of claim 1, wherein the meganuclease DNA-binding domain comprises a recombinant meganuclease having altered specificity for at least one recognition sequence half-site relative to a wild-type I-CreI meganuclease, comprising:
a polypeptide having at least 85% sequence similarity to residues 2-153 of the I-CreI meganuclease of SEQ ID NO: 1; and
having specificity for a recognition sequence half-site which differs by at least one base pair from a half-site within an I-CreI meganuclease recognition sequence selected from the group consisting of SEQ ID NO: 2, SEQ ID NO: 3, SEQ ID NO: 4 and SEQ ID NO: 5;
wherein said recombinant meganuclease comprises at least one modification of Table 1 and a modification which reduces or abolishes said endonuclease cleavage activity.

9. The targeted transcriptional effector of claim 8, wherein the modification which reduces or abolishes said endonuclease cleavage activity is Q47E.

10. The targeted transcriptional effector of claim 1, wherein the meganuclease DNA-binding domain comprises a recombinant meganuclease having altered specificity for at least one recognition sequence half-site relative to a wild-type I-MsoI meganuclease, comprising:
a polypeptide having at least 85% sequence similarity to residues 6-160 of the I-MsoI meganuclease of SEQ ID NO: 6; and
having specificity for a recognition sequence half-site which differs by at least one base pair from a half-site within an I-MsoI meganuclease recognition sequence selected from the group consisting of SEQ ID NO: 7 and SEQ ID NO: 8;
wherein said recombinant meganuclease comprises at least one modification of Table 2 and a modification which reduces or abolishes said endonuclease cleavage activity.

11. The targeted transcriptional effector of claim 10, wherein the modification which reduces or abolishes said endonuclease cleavage activity is D22N.

12. The targeted transcriptional effector of claim 1, wherein the meganuclease DNA-binding domain comprises a recombinant meganuclease having altered specificity for a recognition sequence relative to a wild-type I-SceI meganuclease, comprising:
a polypeptide having at least 85% sequence similarity to residues 3-186 of the I-SceI meganuclease of SEQ ID NO: 9; and
having specificity for a recognition sequence which differs by at least one base pair from an I-SceI meganuclease recognition sequence of SEQ ID NO: 10 and SEQ ID NO: 11;
wherein said recombinant meganuclease comprises at least one modification of Table 3 and a modification which reduces or abolishes said endonuclease cleavage activity.

13. The targeted transcriptional effector of claim 12, wherein the modification which reduces or abolishes said endonuclease cleavage activity is D44N or D145N.

14. The targeted transcriptional effector of claim 1, wherein the meganuclease DNA-binding domain comprises a recombinant meganuclease having altered specificity for at least one recognition sequence half-site relative to a wild-type I-CeuI meganuclease, comprising:
a polypeptide having at least 85% sequence similarity to residues 5-211 of the I-CeuI meganuclease of SEQ ID NO: 12; and
having specificity for a recognition sequence half-site which differs by at least one base pair from a half-site within an I-CeuI meganuclease recognition sequence selected from the group consisting of SEQ ID NO: 13 and SEQ ID NO: 14;
wherein said recombinant meganuclease comprises at least one modification of Table 4 and a modification which reduces or abolishes said endonuclease cleavage activity.

15. The targeted transcriptional effector of claim 14, wherein the modification which reduces or abolishes said endonuclease cleavage activity is E66Q.

16. The targeted transcriptional effector of claim 1, wherein the meganuclease DNA-binding domain comprises a recombinant meganuclease having altered specificity for at least one recognition sequence half-site relative to a wild-type I-CreI meganuclease, comprising:
a polypeptide having at least 85% sequence similarity to residues 2-153 of the I-CreI meganuclease of SEQ ID NO: 1; and
having specificity for a recognition sequence half-site which differs by at least one base pair from a half-site within an I-CreI meganuclease recognition sequence selected from the group consisting of SEQ ID NO: 2, SEQ ID NO: 3, SEQ ID NO: 4 and SEQ ID NO: 5;
wherein:
(1) specificity at position −1 has been altered:
(a) to a T on a sense strand by a modification selected from the group consisting of Q70, C70, L70, Y75, Q75, H75, H139, Q46 and H46;
(b) to an A on a sense strand by a modification selected from the group consisting of Y75, L75, C75, Y139, C46 and A46;
(c) to a G on a sense strand by a modification selected from the group consisting of K70, E70, E75, E46 and D46;
(d) to a C on a sense strand by a modification selected from the group consisting of H75, R75, H46, K46 and R46; or
(e) to any base on a sense strand by a modification selected from the group consisting of G70, A70, S70 and G46; and/or
(2) specificity at position −2 has been altered:
(a) to an A on a sense strand by a modification selected from the group consisting of Q70, T44, A44, V44, I44, L44, and N44;
(b) to a C on a sense strand by a modification selected from the group consisting of E70, D70, K44 and R44;
(c) to a G on a sense strand by a modification selected from the group consisting of H70, D44 and E44; or
(d) to an A or T on a sense strand by a modification comprising C44; and/or
(3) specificity at position −3 has been altered:
(a) to an A on a sense strand by a modification selected from the group consisting of Q68 and C24;
(b) to a C on a sense strand by a modification selected from the group consisting of E68, F68, K24 and R24;
(c) to a T on a sense strand by a modification selected from the group consisting of M68, C68, L68 and F68;
(d) to an A or C on a sense strand by a modification comprising H68;
(e) to a C or T on a sense strand by a modification comprising Y68; or
(f) to a G or T on a sense strand by a modification comprising K68; and/or
(4) specificity at position −4 has been altered:
(a) to a C on a sense strand by a modification selected from the group consisting of E77 and K26;
(b) to a G on a sense strand by a modification selected from the group consisting of E26 and R77;
(c) to a C or T on a sense strand by a modification comprising S77; or
(d) to a any base on a sense strand by a modification comprising S26; and/or
(5) specificity at position −5 has been altered:
(a) to a C on a sense strand by a modification comprising E42;
(b) to a G on a sense strand by a modification comprising R42;
(c) to an A or G on a sense strand by a modification selected from the group consisting of C28 and Q42; or
(d) to any base on a sense strand by a modification of selected from the group consisting of M66 and K66; and/or
(6) specificity at position −6 has been altered:
(a) to a T on a sense strand by a modification selected from the group consisting of C40, I40, V40, C79, I79, V79, and Q28;
(b) to a C on a sense strand by a modification selected from the group consisting of E40 and R28; or
(c) to a G on a sense strand by a modification comprising R40; and/or
(7) specificity at position −7 has been altered:
(a) to a C on a sense strand by a modification selected from the group consisting of E38, K30 and R30;
(b) to a G on a sense strand by a modification selected from the group consisting of K38, R38 and E30;
(c) to a T on a sense strand by a modification selected from the group consisting of I38 and L38; or
(d) to an A or G on a sense strand by a modification comprising C38; or
(e) to any base on a sense strand by a modification selected from the group consisting of H38, N38 and Q30; and/or
(8) specificity at position −8 has been altered:
(a) to a T on a sense strand by a modification selected from the group consisting of L33, V33, I33, F33 and C33;
(b) to a C on a sense strand by a modification selected from the group consisting of E33 and D33;
(c) to a G on a sense strand by a modification consisting of K33;
(d) to an A or C on a sense strand by a modification comprising R32; or
(e) to an A or G on a sense strand by a modification comprising R33; and/or
(9) specificity at position −9 has been altered:
(a) to a C on a sense strand by a modification comprising E32;
(b) to a G on a sense strand by a modification selected from the group consisting of R32 and K32;
(c) to a T on a sense strand by a modification selected from the group consisting of L32, V32, A32 and C32;
(d) to a C or T on a sense strand by a modification selected from the group consisting of D32 and I32; or
(e) to any base on a sense strand by a modification selected from the group consisting of S32, N32, H32, Q32 and T32.

17. The targeted transcriptional effector of claim 1, wherein the meganuclease DNA-binding domain comprises a recombinant meganuclease having altered specificity for at least one recognition sequence half-site relative to a wild-type I-MsoI meganuclease, comprising:
a polypeptide having at least 85% sequence similarity to residues 6-160 of the I-MsoI meganuclease of SEQ ID NO: 6; and
having specificity for a recognition sequence half-site which differs by at least one base pair from a half-site within an I-MsoI meganuclease recognition sequence selected from the group consisting of SEQ ID NO: 7 and SEQ ID NO: 8;
wherein:
(1) specificity at position −1 has been altered:
(a) to an A on a sense strand by a modification selected from the group consisting of K75, Q77, A49, C49 and K79;
(b) to a T on a sense strand by a modification selected from the group consisting of C77, L77 and Q79; or
(c) to a G on a sense strand by a modification selected from the group consisting of K77, R77, E49 and E79; and/or
(2) specificity at position −2 has been altered:
(a) to an A on a sense strand by a modification selected from the group consisting of Q75, K81, C47, I47 and L47;
(b) to a C on a sense strand by a modification selected from the group consisting of E75, D75, R47, K47, K81 and R81; or
(c) to a G on a sense strand by a modification selected from the group consisting of K75, E47 and E81; and/or
(3) specificity at position −3 has been altered:
(a) to an A on a sense strand by a modification selected from the group consisting of Q72, C26, L26, V26, A26 and I26;
(b) to a C on a sense strand by a modification selected from the group consisting of E72, Y72, H26, K26 and R26; or
(c) to a T on a sense strand by a modification selected from the group consisting of K72, Y72 and H26; and/or
(4) specificity at position −4 has been altered:
(a) to a T on a sense strand by a modification selected from the group consisting of K28, K83 and Q28;
(b) to a G on a sense strand by a modification selected from the group consisting of R83 and K83; or
(c) to an A on a sense strand by a modification selected from the group consisting of K28 and Q83; and/or
(5) specificity at position −5 has been altered:
(a) to a G on a sense strand by a modification selected from the group consisting of R45 and E28;
(b) to a T on a sense strand by a modification comprising Q28; or
(c) to a C on a sense strand by a modification comprising R28; and/or
(6) specificity at position −6 has been altered:
(a) to a T on a sense strand by a modification selected from the group consisting of K43, V85, L85 and Q30;
(b) to a C on a sense strand by a modification selected from the group consisting of E43, E85, K30 and R30; or
(c) to a G on a sense strand by a modification selected from the group consisting of R43, K43, K85, R85, E30 and D30; and/or
(7) specificity at position −7 has been altered:
(a) to a C on a sense strand by a modification selected from the group consisting of E32 and E41;
(b) to a G on a sense strand by a modification selected from the group consisting of R32, R41 and K41;
(c) to a T on a sense strand by a modification selected from the group consisting of K32, M41, L41 and I41; and/or
(8) specificity at position −8 has been altered:
(a) to a T on a sense strand by a modification selected from the group consisting of K32 and K35;
(b) to a C on a sense strand by a modification comprising E32; or
(c) to a G on a sense strand by a modification consisting of K32, K35 and R35; and/or
(9) specificity at position −9 has been altered:
(a) to an A on a sense strand by a modification selected from the group consisting of N34 and H34;
(b) to a T on a sense strand by a modification selected from the group consisting of S34, C34, V34, T34 and A34; or
(c) to a G on a sense strand by a modification selected from the group consisting of K34, R34 and H34.

18-34. (canceled)

35. A nucleic acid encoding the targeted transcriptional effector of claim 1.

36. A method for treating a disease or condition in a subject in need thereof, the method comprising: introducing the nucleic acid of claim 35 into a subject, whereby the polypeptide encoded by the nucleic acid binds to the target site and affects transcription of the gene of interest.

37. A method for treating a disease or condition in a subject in need thereof, the method comprising: introducing the targeted transcriptional effector of claim 1 into a subject, whereby the polypeptide binds to the target site and affects transcription of the gene of interest.
